(19) United States
(12) Patent Application Publication
(10) Pub. No.: US 2004/0171153 A1
(43) Pub. Date: Sep. 2, 2004

(54) Stem cell

(76) Inventors: Peter Andrews, Sheffield (GB); James Walsh, Sheffield (GB); Paul Gokhale, Sheffield (GB)

Correspondence Address: CROWELL & MORING LLP, INTELLECTUAL PROPERTY GROUP, P.O. BOX 14300, WASHINGTON, DC, 20044-4300, US

(21) Appl. No.: 10/472,545

(22) PCT Filed: Mar. 25, 2002
(86) PCT No.: PCT/GB02/01195

(30) Foreign Application Priority Data
Mar. 23, 2001 (GB) 0107299.0
Mar. 23, 2001 (GB) 0107296.6
Apr. 17, 2001 (GB) 0109346.7

(51) Int. Cl. C12N 15/85; C12N 5/08

(52) U.S. Cl. 435/455; 435/366

(57) ABSTRACT

There is provided a method to modulate the differentiation state of embryonic stem cells in culture by the providing ligands which bind receptors in the Notch and Wnt pathways.

The invention relates to a method to modulate the differentiation state of embryonic stem cells.

During mammalian development those cells that form part of the embryo up until the formation of the blastocyst are said to be totipotent (e.g. each cell has the developmental potential to form a complete embryo and all the cells required to support the growth and development of said embryo). During the formation of the blastocyst, the cells that comprise the inner cell mass are said to be pluripotential (e.g. each cell has the developmental potential to form a variety of tissues).

Embryonic stem cells (ES cells, those with pluripotentiality) may be principally derived from two embryonic sources. Cells isolated from the inner cell mass are termed embryonic stem (ES) cells. In the laboratory mouse, similar cells can be derived from the culture of primordial germ cells isolated from the mesenteries or genital ridges of days 8.5-12.5 post coitum embryos. These would ultimately differentiate into germ cells and are referred to as embryonic germ cells (EG cells). Each of these types of pluripotential cell has a similar developmental potential with respect to differentiation into alternate cell types, but possible differences in behaviour (eg with respect to imprinting) have led to these cells to be distinguished from one another. Hereinafter embryonic stem cells will encompass both these stem cell-types.

Typically ES cell cultures have well defined characteristics. These include, but are not limited to; maintenance in culture for at least 20 passages when maintained on fibroblast feeder layers; produce clusters of cells in culture referred to as embryoid bodies; the ability to differentiate into multiple cell types in monolayer culture; and express ES cell specific markers.

Until very recently, in vitro culture of human ES cells was not possible. The first indication that conditions may be determined which could allow the establishment of human ES cells in culture is described in WO96/22362. The application describes cell lines and growth conditions which allow the continuous proliferation of primate ES cells which exhibit a range of characteristics or markers which are associated with stem cells having pluripotent characteristics.

More recently Thomson et al (1998) have published conditions in which human ES cells can be established in culture. The above characteristics shown by primate ES cells are also shown by the human ES cell lines. In addition the human cell lines show high levels of telomerase activity, a characteristic of cells which have the ability to divide continuously in culture in an undifferentiated state. Another group (Reubinoff et. al., 2000) have also reported the derivation of human ES cells from human blastocysts. A third group (Shamblott et. al., 1998) have described EG cell derivation.

A feature of ES cells is that, in the presence of fibroblast feeder layers, they retain the ability to divide in an undifferentiated state for several generations. If the feeder layers are removed then the cells differentiate. The differentiation is often to neurones or muscle cells but the exact mechanism by which this occurs and its control remain unsolved. It would be desirable to have a reliable culture system which does not require the presence of fibroblast feeder cells but includes the addition of a factor(s) which maintain ES cells in an undifferentiated state. A prerequisite to the successful exploitation of ES cells in tissue engineering is to provide a reliable and defined cell culture system which can be used to control the differentiation of ES cells into a selected cell-type. The identification of gene targets involved in maintaining ES cells as ES cells and the identification of gene targets involved in differentiation will facilitate this objective.

We have identified a regulatory pathway involved in the mechanism by which ES cells are maintained as ES cells in culture and which also influences the differentiation of said cells in culture. The regulatory pathway comprises two families of genes referred to as Notch and Wnt.

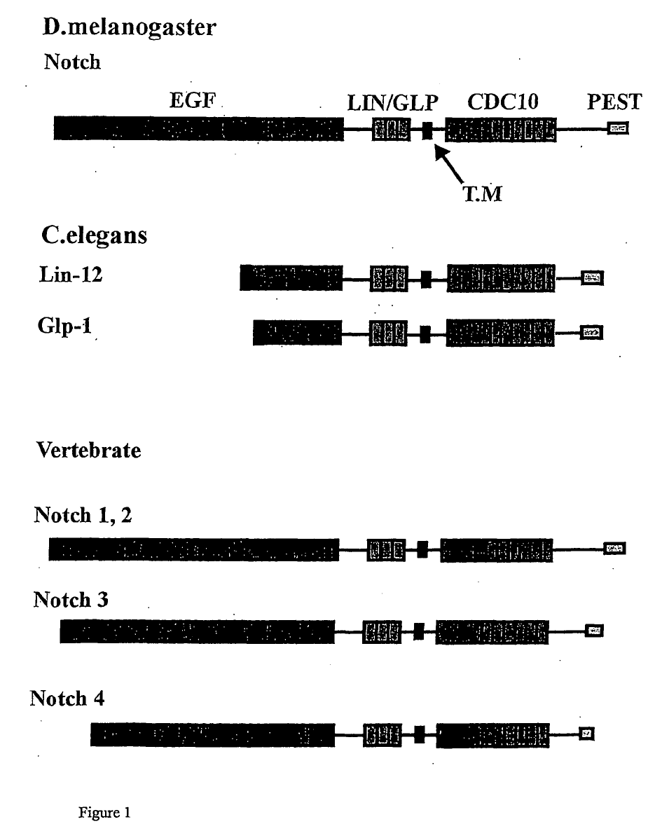

The Notch gene is a Drosophila prototype for a family of homologues found in diverse species, encoding large, single-span, transmembrane receptors (reviewed in Weinmaster, 199-7). Within the extracellular domain, located distally from the transmembrane region, are found multiple (10-36), tandem arrays of epidermal growth factor-like repeats (Wharton et al., 1985; Kopezynski et al., 1988). More proximally are found 3 cysteine-rich, Lin-12/Notch repeats and two conserved cysteine residues. The intracellular domain contains, from proximal to distal with respect to the transmembrane region, a subtransmembrane region (STR), six ankyrin repeats and a region rich in proline, glutamic acid, serine and threonine (PEST). The generic Notch structure is illustrated in FIG. 1.

Wnt genes encode diffusible, extracellular signalling molecules of around 350-400 amino acids in length, defined by a characteristic pattern of conserved cysteine residues, along with other invariant amino acids (see http://www.stanford.edu/˜rnusse/wntwindow.html.

In the 1970s, the wingless (wg¹) mutation of Drosophila melanogaster was described, in which affected individuals showed aberrant wing and haltere development (Sharma, 1973; Sharma and Chopra, 1976). When the gene disrupted by this mutation was subsequently identified, the predicted 468aa peptide sequence exhibited remarkable similarity to that of a murine gene, in-1 (Cabrera et al., 1987; Rijsewijk et al., 1987), including an identical pattern of 23 conserved cysteine residues. int-1 had earlier been identified as a common integration site of the murine mammary tumour virus, and a likely cellular oncogene (Nusse and Varmus, 1982; van Ooyen and Nusse, 1984). Thus, the two prototypic members of the Wnt gene family were described. Since that time, numerous homologues of wingless/int-1 have been identified in divergent organisms, including Caenorhabditis elegans, Drosophila melanogaster, Xeizopus laevis, chicken, mouse and humans (reviewed in Cadigan and Nusse, 1997; Wodarz and Nusse, 1998). Lower organisms appear to possess a limited repertoire of Wnt genes in comparison to higher organisms, presumably reflecting their lesser developmental complexity. Additionally, vertebrates appear to express multiple, closely related orthologues of certain Wnts. The Wnt family is composed of more than 60 members, with 14 human homologues alone. Well-documented roles exist for Hint signalling in a variety of developmental processes, including cell fate specification and patterning within the central nervous system.

Wnt ligands interact with membrane-bound receptors of the frizzled family, leading to activation of a cytoplasmic protein, Dishevelled. Dishevelled inhibits Notch activation (2) and also inhibits the activity of an Axin-APC-GSK-3b complex, promoting formation of a bipartite transcriptional activator comprising b-catenin and TCF (4). Wnt signalling may be antagonised by extracellular molecules that compete for Wnt binding, including frizzled related proteins (FRP), Wnt inhibitory factors (WIF), Dickkopf and Cerberus. Expression of Wnt target genes may also be regulated by other proteins that bind to and alter the function of TCF. CREB-Binding Protein (CBP) exhibits a mutually antagonistic binding affinity for TCF with b-catenin and converts TCF into a repressor of target genes (8). Additionally, Notch activation may induce transcriptional repression by TCF, even in the presence of b-catenin, through expression of the TLE class of putative target genes (5,7).

As a model system to test the involvement of Notch and Wnt genes in the differentiation of ES cells we have used embryonal carcinoma cells which are stem cells of teratocarcinomas. The stem cells of early embryos and the stem cells of teratocarcinomas have been demonstrated experimentally to be capable of substituting for one another in their respective roles. Thus, an embryonic stem cell introduced to a syngeneic host may give rise to a teratocarcinoma containing all of the elements that would be found in a spontaneous tumour of this type (Mintz et al 1978). Likewise, embryonal carcinoma cells derived from a spontaneous germ cell carcinoma may participate in embryonic development, and generate normal somatic tissue following injection into a blastocyst (Brinster 1974; Mintz and Illmensee 1975; Papaioannou et al 1975). This clearly demonstrates that murine EC cells may respond to developmental cues in an appropriate manner, and that their differentiation may provide information pertinent to normal embryogenesis. Similarly, human EC cells may provide an insight into the processes that regulate human development.

The TERA2 cell line was derived from a lung metastasis of a human teratocarcinoma in the mid 1970s (Fogh and Trempe, 1975). Morphologically, TERA2 cultures are quite divergent from the characteristic EC phenotype and display significant heterogeneity, suggesting that these cells undergo spontaneous differentiation (Andrews et al., 1980). However, a tumour containing both embryonal carcinoma cells and differentiated derivatives was produced following injection of TERA2 into a nude mouse host (Andrews et al., 1983a; Andrews et al., 1983b; Andrews et al., 1984). A cell line established from the EC component of this tumour, named NTERA2, closely resembled and maintained the characteristic EC phenotype in culture and, unlike the parent line, was able to produce teratocarcinoma in nude mice with high frequency (Andrews et al., 1983a; Andrews et al., 1983b; Andrews et al., 1984). Additionally, various subclones of NTERA2 exhibit the ability to differentiate extensively in vitro following treatment with chemical inducers (eg retinoic acid (RA), HMBA) (Andrews, 1984; Andrews et al., 1986).

The expression of human Notch homologues were examined in NTERA2 to determine their involvement in ES cell differentiation.

We have discovered that members of the Notch gene family, Notch1 (Genbank accession number AF308602), Notch2 (Genbank accession number NM_{—}024408) and Notch3 (Genbank accession number NM_{—}000435) are expressed in EC cells and NTERA2 cells. Notch1 expression was detected as a mRNA band of around 7 Kb in both EC and differentiated cultures of NTERA2. Notch3, like Notch1, was examined in EC cells. A transcript of around 8 Kb was readily detected in all samples. The endoderm-specific Notch4 (Genbank accession number XM_{—}004207) was not.

All three Notch homologues expressed by NTERA2 showed altered transcription during differentiation in response to retinoic acid. In each case, however, these changes were modest and expression was evident in both EC and differentiated cultures. The role of the Notch pathway in directing EC/ES differentiation may thus depend to a greater extent on the level of signalling activation rather than the abundance of the receptors. In order to investigate this possibility, the expression of candidate ligands for Notch receptors were examined. For example, dlk (Genbank accession number U15979) was detected at high levels in EC cultures, but its expression was almost extinguished by 3 days following RA treatment. Low levels were also observed through 7 and 14 days post-RA. However, by 21 days, dlk was up-regulated to the level seen in EC cultures. These profound changes may reflect an important role for dlk and other DSL ligands in regulating EC/ES differentiation through altered Notch signalling activation. This data is suggestive that the Notch signalling pathway is involved in regulating EC cell differentiation and, by extrapolation, human ES cell differentiation.

A degenerate PCR strategy was used to investigate the possible expression of novel Wnt genes in the NTERA2 system. The expression of a single Wnt gene, Wnt-13, was detected in NTERA2. Wnt-13 was absent in EC cells, but showed induction and subsequent up-regulation following both retinoic acid and HMBA treatment. Both of these agents bring about extensive differentiation of NTERA2, accompanied by the loss of typical human EC surface markers.

We have examined the expression of components of the Wnt pathway and of transcripts corresponding to other proteins known to interact with Wnt signalling in NTERA2 cells. These cells are a model system for aspects of human embryogenesis and differentiate extensively in vitro in response to chemical inducers. Among the cell types produced following retinoic acid treatment are functional, post-mitotic, CNS neurons (1,6,17).

The modulation of the Notch and Wnt signalling pathways may facilitate manipulation of embryonic stem cell differentiation. The term modulation refers to either the maintenance of embryonic stem cells as embryonic stem cells or the facilitation of differentiation of embryonic stem cells along defined cell lineages.

According to an aspect of the invention there is provided a method to modulate the phenotype of an embryonic stem cell comprising contacting said cell with a ligand binding domain of a polypeptide wherein said domain binds its cognate receptor expressed by said cell to modulate said phenotype.

According to a further aspect of the invention there is provided a method to modulate the differentiation of an embryonic stem cell comprising:

i) providing a culture of embryonic stem cells;

ii) providing at least one ligand, or the active binding fragment thereof, capable of binding its cognate receptor polypeptide expressed by said embryonic stem cell;

iii) forming a culture comprising embryonic stem cells and said ligand; and

iv) growing said cell culture.

In a preferred method of the invention said ligand is encoded by a nucleic acid molecule selected from the group consisting of:

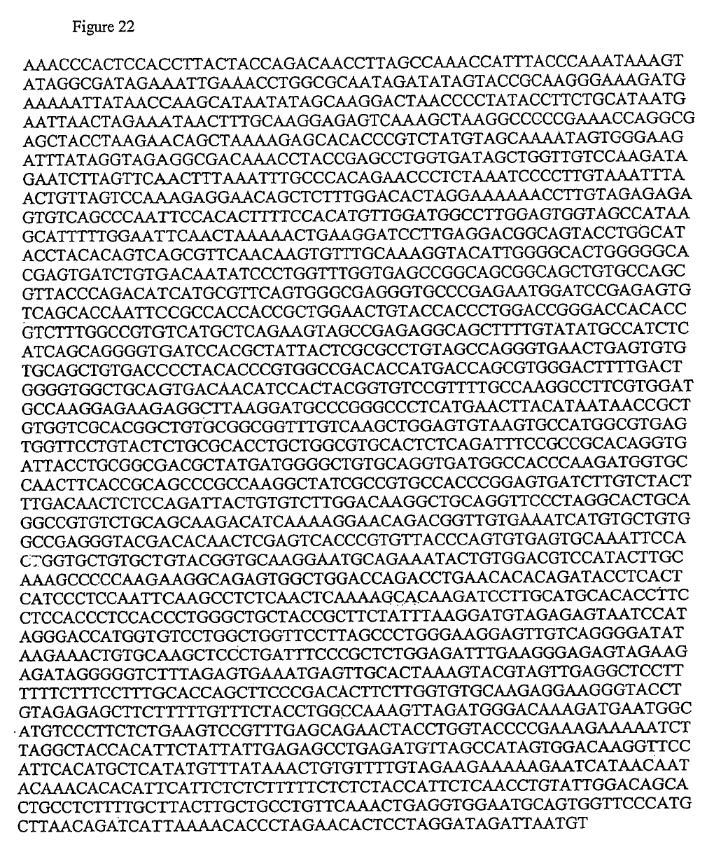

i) a nucleic acid molecule as represented in FIG. 22;

ii) a nucleic acid molecule which hybridises to the nucleic acid in (i) and which encodes a ligand capable of binding a Wnt receptor; and

iii) nucleic acid molecules which are degenerate as a result of the genetic code to the sequences defined in (i) and (ii) above.

In a preferred method of the invention said ligand is selected from the group consisting of: WNT 1; WNT 2, WNT 3; WNT 4; WNT 5A; WNT 6; WNT 7A; WNT 8B; WNT 1013; W 11; WNT 14; WNT 16.

In a further preferred method of the invention said ligand is WNT 13.

In an alternative preferred method of the invention said ligand is encoded by a nucleic acid molecule selected from the group consisting of:

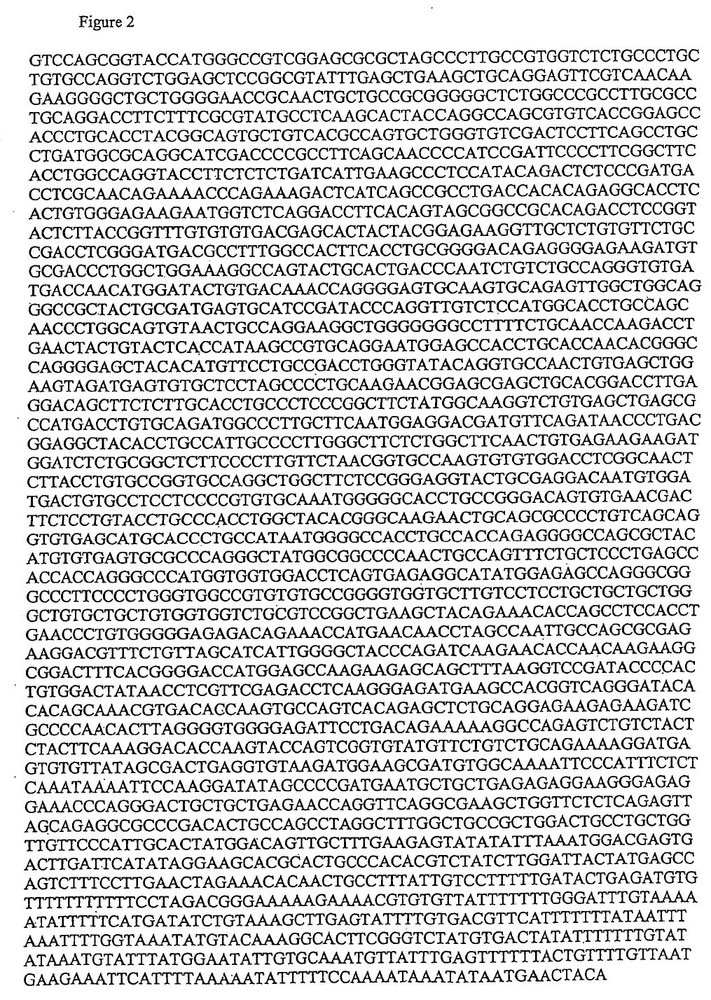

i) a nucleic acid molecule as represented in FIGS. 2, 4, 5, 7, 10, 12, 14, 16, or 18.

ii) a nucleic acid molecule which hybridises to the nucleic acid in (i) and which encodes a ligand capable of modulating embryonic stem cell differentiation; and

iii) nucleic acid molecules which are degenerate as a result of the genetic code to the sequences defined in (i) and (ii) above.

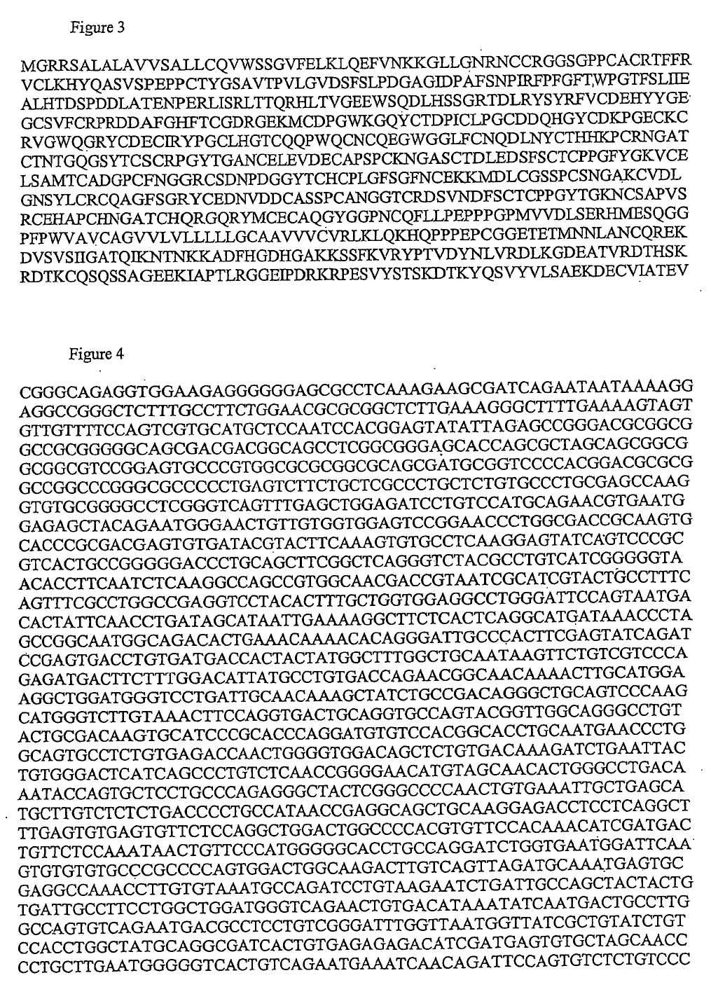

In a further preferred method of the invention said ligand is selected from the group represented by the amino acid sequences in FIG. 3, 6, 8, 9, 11, 13, 15, 17, 19, or polypeptide variants thereof.

Polypeptide variants are polypeptide sequences having at least 75% identity with the polypeptide sequences as herein disclosed, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequences illustrated herein.

In a further preferred method of the invention said cells are induced to differentiate by the addition of at least one agent selected from the group consisting of: retinoic acid; HMBA; bone morphogenetic proteins; bromodeoxyuridine; lithium; sonic hedgehog.

Optionally the inducing agent and the ligand are added simultaneously to a culture of embryonic stem cells. Alternatively, the ligand is added before addition of said inducing agent.

According to a further aspect of the invention there is provided a method for modulating the differentiation of embryonic stem cells comprising:

i) providing a cell transfected with a nucleic acid molecule selected from the group consisting of:

a) a nucleic acid molecule as represented in FIGS. 2, 4, 5, 7, 10, 12, 14, 16, 18.

b) a nucleic acid molecule which hybridises to the nucleic acid in (ii) and which encodes a ligand capable of modulating embryonic stem cell differentiation; and

c) nucleic acid molecules which are degenerate as a result of the genetic code to the sequences defined in (a) and (b) above.

ii) forming a culture comprising the cell identified in (i) above with an embryonic stem cell; and

iii) growing said culture under conditions suitable for the maintenance and/or differentiation of said embryonic stem cell.

According to a yet further aspect of the invention there is provided a method for modulating the differentiation of embryonic stem cells comprising:

i) providing a cell transfected with a nucleic acid molecule selected from the group comprising:

a) a nucleic acid molecule as represented by the sequence in FIG. 22;

b) a nucleic acid molecule which hybridises to the nucleic acid in (a) and which encodes a ligand capable of binding a Wnt receptor; and

c) nucleic acid molecules which are degenerate as a result of the genetic code to the sequences defined in (a) and (b) above.

ii) forming a culture comprising a cell as identified in (i) above with an embryonic stem cell; and

iii) growing said culture under conditions suitable for the maintenance and/or differentiation of embryonic stem cells.

In a preferred method of the invention said cell expresses Wnt-13.

Optionally the cells expressing the ligand(s) are mixed with a culture of undifferentiated embryonic stem cells. This is followed by addition of the inducing agent (eg retinoic acid; HMBA, bone morphogenetic proteins; bromodeoxyuridine; lithium; sonic hedgehog).

In a preferred method of the invention said nucleic acid molecule hybridises under stringent hybridisation conditions to the nucleic acid molecules represented in (a), (b) or (c) above.

Stringent hybridisation or washing conditions are well known in the art. For example, nucleic acid hybrids that are stable after washing in 0.1×SSC, 0.1% SDS at 60° C. It is well known in the art that optimal hybridisation conditions can be calculated if the sequence of the nucleic acid is known. For example, hybridisation conditions can be determined by the GC content of the nucleic acid subject to hybridisation. Please see Sambrook et al (1989) Molecular Cloning; A Laboratory Approach. A common formula for calculating the stringency conditions required to achieve hybridisation between nucleic acid molecules of a specified homology is:

Tₘ=81.5° C.+16.6Log [Na⁺+]+0.41 [%G+C]−0.63 (%formamide)

In a further preferred method of the invention the nucleic acid molecule is genomic DNA or cDNA.

In a preferred method of the invention the nucleic acid molecule encodes a ligand of human origin.

In a further preferred method of the invention said embryonic stem cells are of human origin.

In a yet further preferred method of the invention the cell transfected with the nucleic acid according to the invention is a mammalian cell. Preferably the cell is selected from the following group: a chinese hamster ovary cell; murine primary fibroblast cell; human primary fibroblast cell; transformed mouse fibroblast cell-line STO.

According to a further aspect of the invention there is provided a method for inhibiting the differentiation of embryonic stem cells or embryonal carcinoma cells comprising:

i) providing at least one polypeptide, or active fragment thereof, wherein said polypeptide is an inhibitor of the Wnt signalling pathway.

ii) forming a culture comprising the polypeptide identified in (i) above with an embryonic stem cell; and

iii) growing said culture under conditions suitable for the maintenance of embryonic stem cells in an undifferentiated state.

In a preferred method of the invention said inhibitor of Wnt signalling is selected from the group comprising the active binding fragments thereof of the following polypeptides: frizzled related polypeptides (FRP); Wnt Inhibitory Factors (WIF); Dickkopf; Cerebrus.

In a further preferred method of the invention said inhibitor of Wnt signalling is selected from the group comprising the active binding fragments thereof of the following polypeptides: SFRP1; SFRP4; FRZB; SFRP2; FZD1; FZD2; FZD9; FZD3; FZD5; FZD4; FZD6; FZD7; DVL2; DVL3; GSK3B; AXIN1; APC; TCF1; WIF-1; CER 1; DKK1-4; SARP 2; SARP 3.

According to a further aspect of the invention there is provided a method for inhibiting the differentiation of embryonic stem cells or embryonal carcinoma cells comprising:

i) providing a cell transfected with a nucleic acid molecule selected from the group consisting of:

a) a nucleic acid molecule encoding a Wnt inhibitory polypeptide;

b) a nucleic acid molecule which hybridises to the nucleic acid in (a) and which encodes a polypeptide capable of inhibiting Wnt signalling; and

c) nucleic acid molecules which are degenerate as a result of the genetic code to the sequences defined in (a) and (b) above.

ii) contacting the cell of (i) above with a culture of embryonic stem cells; and

iii) growing said culture under conditions suitable for the maintenance of embryonic stem cells in an undifferentiated state.

In a preferred method of the invention said cells express at least one Wnt inhibitory polypeptide selected from the group comprising the active binding fragments thereof of the following polypeptides: frizzled related polypeptides (FLIP); Wnt Inhibitory Factors (WIF); Dickkopf; Cerebrus. Preferably said cells express at least one Wnt inhibitory polypeptide selected from the group comprising the active binding fragments thereof of the following polypeptides: SFRP1; SFRP4; FRZB; SFRP2; FZD1; FZD2; FZD9; FZD3; FZD5; FZD4; FZD6; FZD7; DVL2; DVL3; GSK3B; AXIN1; APC; TCF1; WIF-1; CER-1; DKK1-4

In a further preferred method of the invention the nucleic acid molecule is encoded by a nucleic acid molecule which hybridises under stringent hybridisation conditions to the nucleic acid molecules represented in (a), (b) or (c) above. Preferably said inhibitors are human.

According to a further aspect of the invention there is provided a vector comprising the nucleic acid molecule according to the invention. Preferably the vector is an expression vector adapted for the expression of the polypeptide encoded by said nucleic acid molecule.

Typically said adaptation includes, by example and not by way of limitation, the provision of transcription control sequences (promoter sequences) which mediate cell/tissue specific expression. These promoter sequences may be cell/tissue specific, inducible or constitutive.

Promoter is an art recognised term and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences and is therefore position independent). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to a number of environmental cues which include, by example and not by way of limitation, intermediary metabolites (eg glucose, lipids), environmental effectors (eg light, heat,).

Promoter elements also include so called TATA box and RNA polymerase initiation selection (RIS) sequences which function to select a site of transcription initiation. These sequences also bind polypeptides which function, inter alia, to facilitate transcription initiation selection by RNA polymerase.

Adaptations also include the provision of selectable markers and autonomous replication sequences which both facilitate the maintenance of said vector in either the eukaryotic cell or prokaryotic host. Vectors which are maintained autonomously are referred to as episomal vectors. Episomal vectors are desirable since these molecules can incorporate large DNA fragments (30-50 kb DNA). Episomal vectors of this type are described in WO98/07876. Alternatively, the vector is an integrating vector.

Adaptations which facilitate the expression of vector encoded genes include the provision of transcription termination/polyadenylation sequences. This also includes the provision of internal ribosome entry sites (RES) which function to maximise expression of vector encoded genes arranged in bicistronic or multi-cistronic expression cassettes.

These adaptations are well known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N. Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc. (1994).

Conventional methods to introduce DNA or vector DNA into cells are well known in the art and typically involve the use of chemical reagents, cationic lipids or physical methods. Chemical methods which facilitate the uptake of DNA by cells include the use of DEAE-Dextran (Vaheri and Pagano Science 175: p434). DEAE-dextran is a negatively charged cation which associates and introduces the DNA into cells but which can result in loss of cell viability. Calcium phosphate is also a commonly used chemical agent which when co-precipitated with DNA introduces the DNA into cells (Graham et al Virology (1973) 52: p456).

The use of cationic lipids (eg liposomes, Felgner (1987) Proc.Natl.Acad.Sci USA, 84:p7413) has become a common method since it does not have the degree of toxicity shown by the above described chemical methods. The cationic head of the lipid associates with the negatively charged nucleic acid backbone of the DNA to be introduced. The lipid/DNA complex associates with the cell membrane and fuses with the cell to introduce the associated DNA into the cell. Liposome mediated DNA transfer has several advantages over existing methods. For example, cells which are recalcitrant to traditional chemical methods are more easily transfected using liposome mediated transfer.

More recently still, physical methods to introduce DNA have become effective means to reproducibly transfect cells. Direct microinjection is one such method which can deliver DNA directly to the nucleus of a cell (Capecchi (1980) Cell, 22:p479). This allows the analysis of single cell transfectants. So called “biolistic” methods physically shoot DNA into cells and/or organelles using a particle gun (Neumann (1982) EMBO J, 1: p841). Electroporation is arguably the most popular method to transfect DNA. The method involves the use of a high voltage electrical charge to momentarily permeabilise cell membranes making them permeable to macromolecular complexes. However physical methods to introduce DNA do result in considerable loss of cell viability due to intracellular damage. These methods therefore require extensive optimisation and also require expensive equipment.

More recently still a method termed immunoporation has become a recognised techinque for the introduction of nucleic acid into cells, see Bildirici et al, Nature 405, 769. The technique involves the use of beads coated with an antibody to a specific receptor. The transfection mixture includes nucleic acid, typically vector DNA, antibody coated beads and cells expressing a specific cell surface receptor. The coated beads bind the cell surface receptor and when a shear force is applied to the cells the beads are stripped from the cell surface. During bead removal a transient hole is created through which nucleic acid and/or other biological molecules, eg polypeptides, can enter. Transfection efficiency of between 40-50% is achievable depending on the nucleic acid used.

Other non-liposome based, chemical transfectant agents have become available, for example ExGen500 (polyethylenimine), produced by MBI Fermentas. ExGen500 is particularly effective for transfection of human ES cells (Eiges, 2001).

According to a further aspect of the invention there is provided a method for the production of the polypeptide encoded by the nucleic acid molecule according to the invention comprising:

i) providing a cell transformed/transfected with a nucleic acid molecule according to the invention;

ii) growing said cell in conditions conducive to the manufacture of said polypeptide; and

i) purifying said polypeptide from said cell, or its growth environment.

In a preferred method of the invention said nucleic acid molecule is the vector according to the invention.

In a further preferred method of the invention said vector encodes, and thus said recombinant polypeptide is provided with, a secretion signal to facilitate purification of said polypeptide.

According to a further aspect of the invention there are provided host cells which have been transformed/transfected with the vector according to the invention, so as to include at least part of the polypeptide according to the invention, so as to permit expression of at least the functional part of the polypeptide encoded by said nucleic acid molecule.

Ideally said host cells are eukaryotic cells, for example, insect cells such as cells from a species Spodoptera frugiperda using the baculovirus expression system.

According to a further aspect of the invention there is provided a therapeutic cell composition comprising differentiated or differentiating embryonic stem cells derived by the method according to the invention. Preferably said composition is for

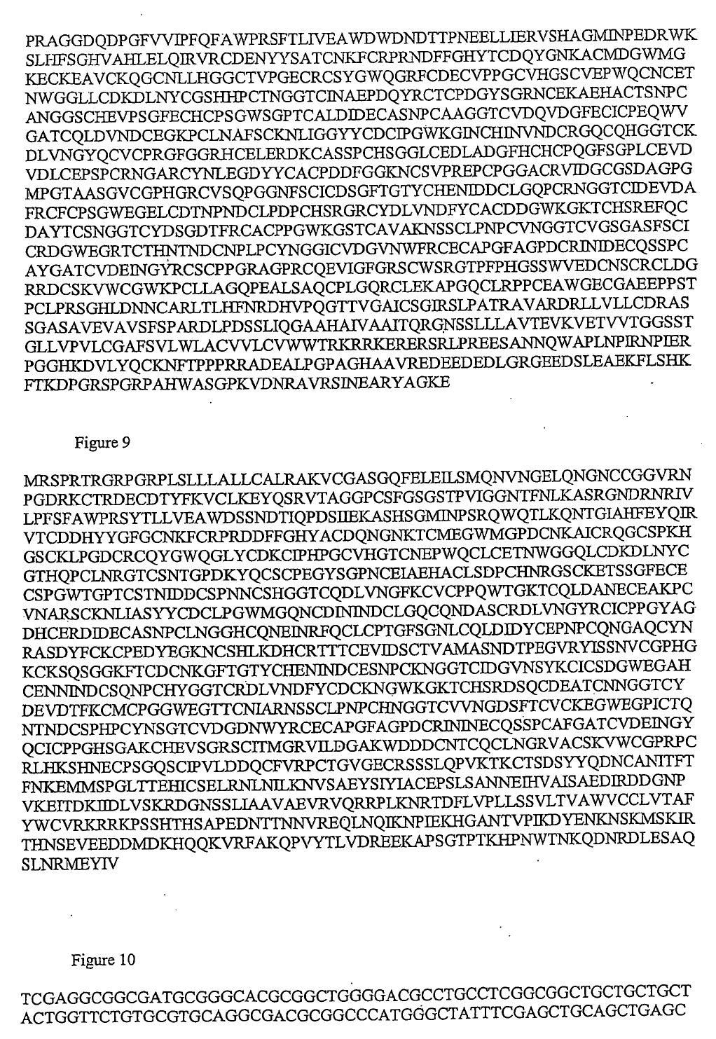

FIG. 9 is the amino acid sequence of murine notch ligand jagged 1;

FIG. 10 is the nucleic acid sequence of murine notch ligand jagged 2;

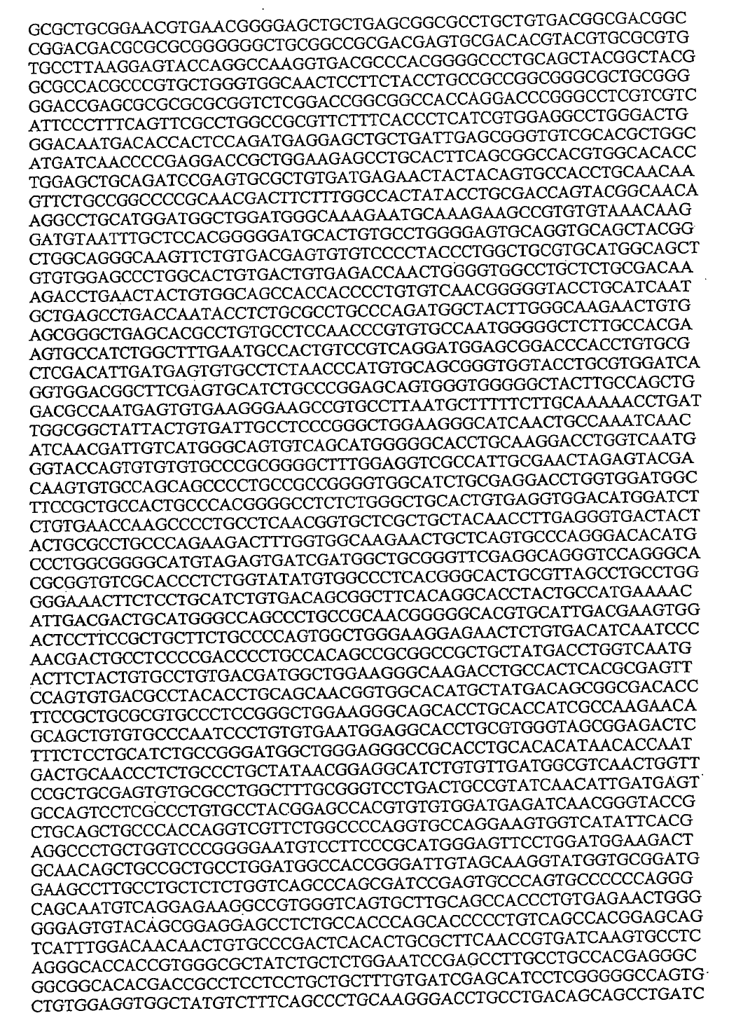

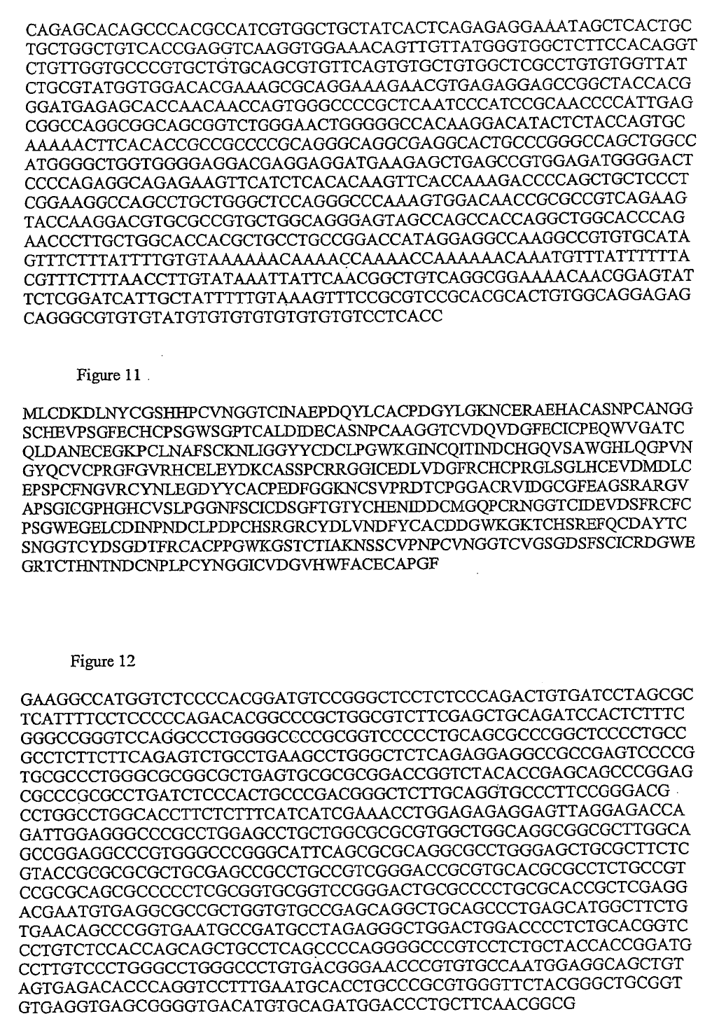

FIG. 11 is the amino acid sequence of murine notch ligand jagged 2;

FIG. 12 is the nucleic acid sequence of human notch ligand delta-like 3 (DLL3);

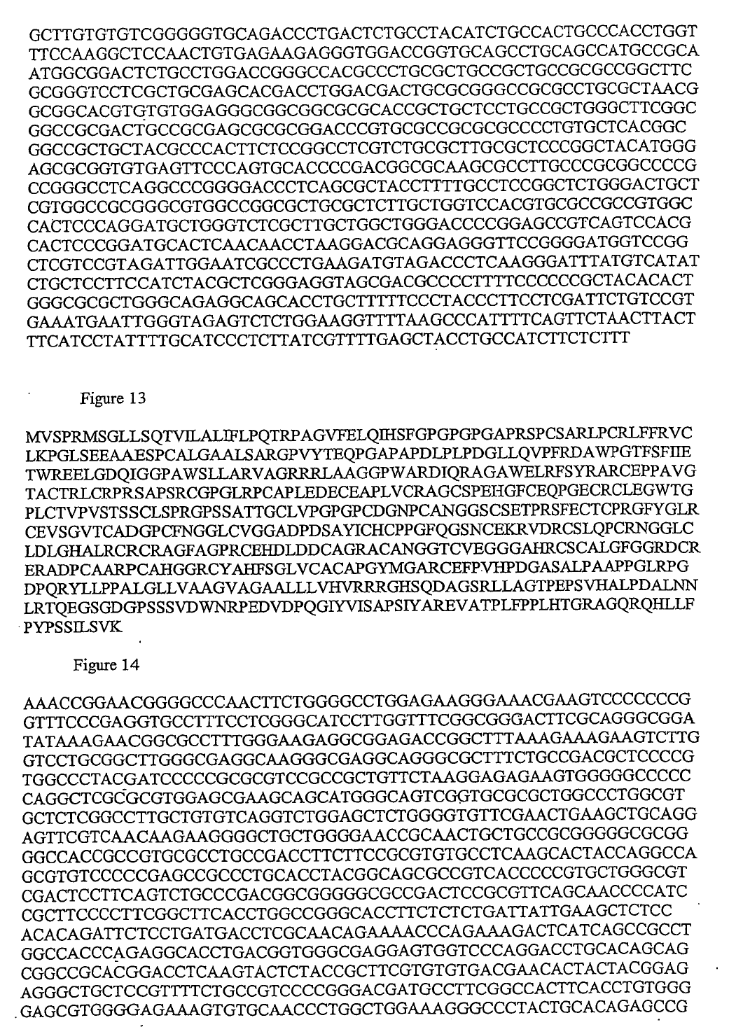

FIG. 13 is the amino acid sequence of human notch ligand delta-like 3 precursor polypeptide;

FIG. 14 is the nucleic acid sequence of human notch ligand delta-1 (DLL1);

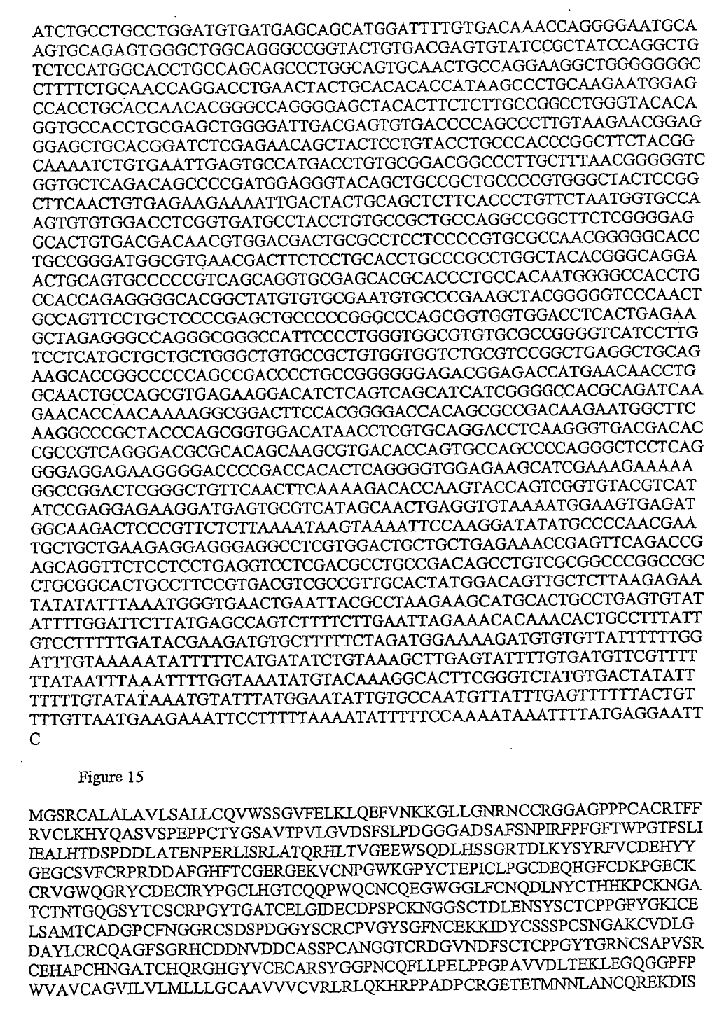

FIG. 15 is the amino acid sequence of murine notch ligand delta-like 1;

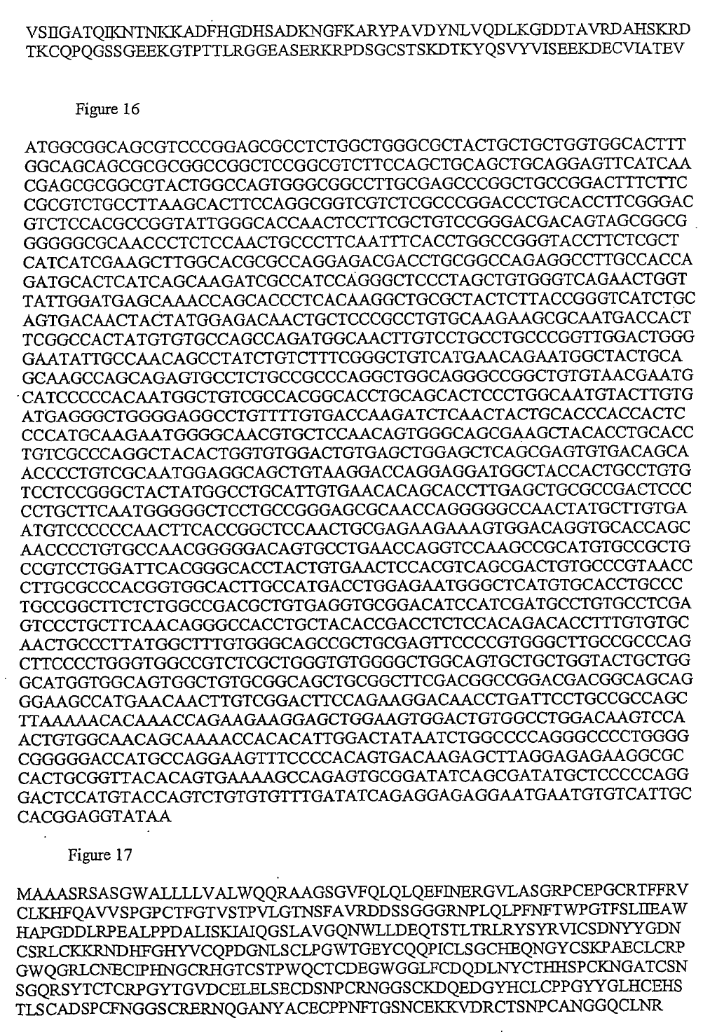

FIG. 16 is the nucleic acid sequence of human notch ligand delta-like 4 (DLL4);

FIG. 17 is the amino acid sequence of human notch ligand delta-like 4 (DLL4);

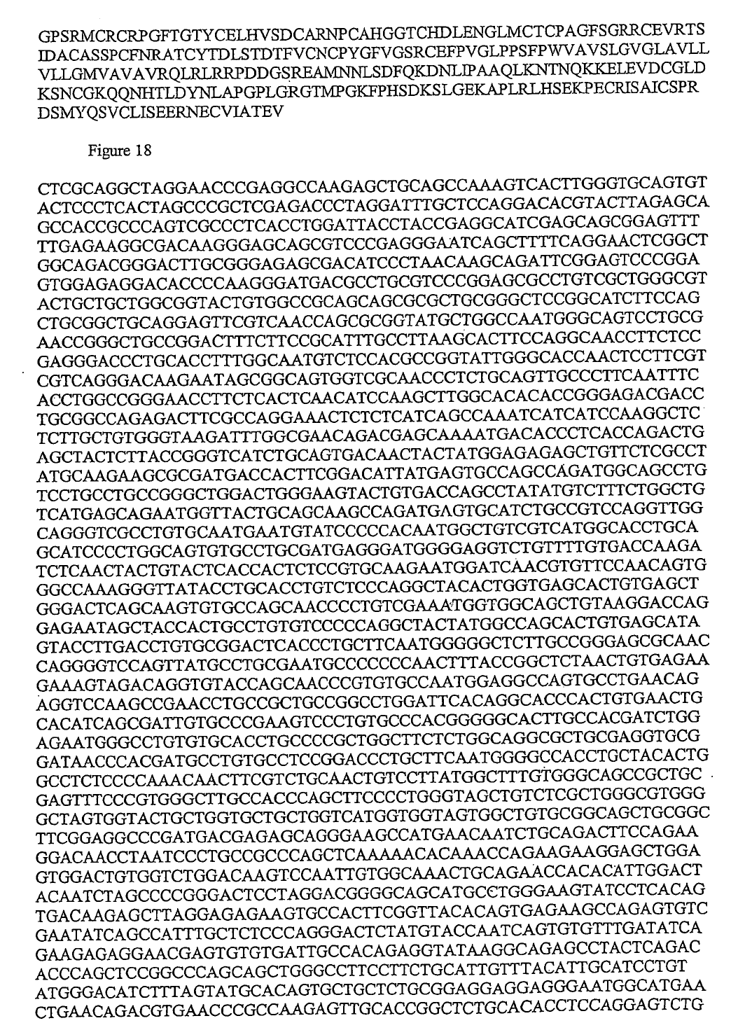

FIG. 18 is the nucleic acid sequence of murine notch ligand delta-like 4 (DLL4);

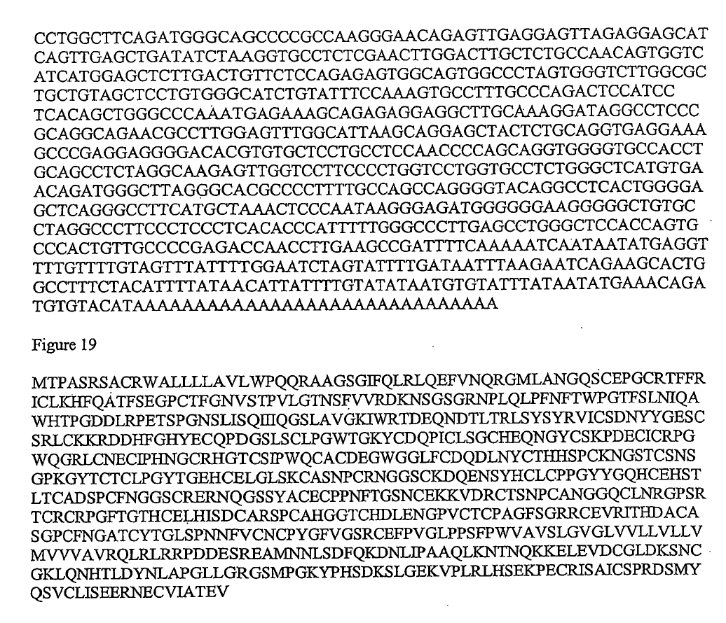

FIG. 19 is the amino acid sequence of murine notch ligand delta-like 4 (DLL4);

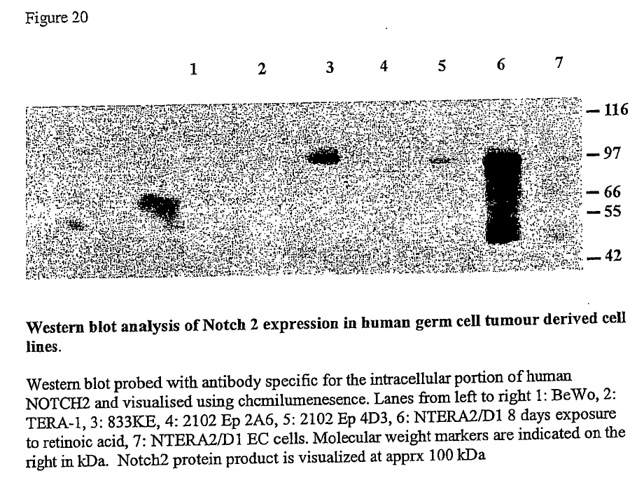

FIG. 20 is a western blot of cell extracts of various EC cell-lines probed with Notch2 antisera;

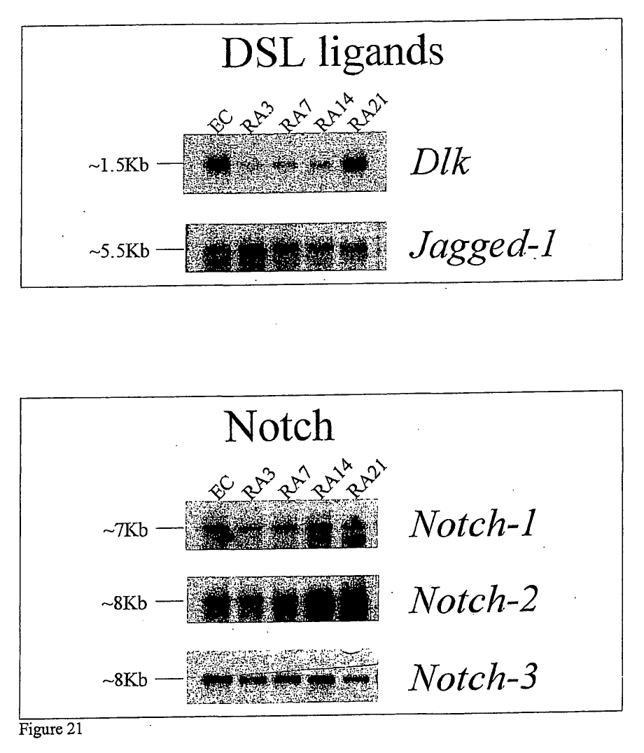

FIG. 21 represents northern blot analysis of the expression patterns of notch genes (Notch 1,2,3) and notch ligands (Dlk, jagged 1) in EC cells and EC cells treated with retinoic acid (RA); use in the treatment of: Parkinson's disease; Huntington's disease; motor neurone disease; heart disease; diabetes; liver disease (eg cirrhosis); renal disease; AIDS.

According to a further aspect of the invention there is provided a method of treatment of an animal comprising administering a cell composition comprising embryonic stem cells which have been induced to differentiate into at least one cell-type.

According to a yet further aspect of the invention there is provided condition medium obtained by culturing embryonic stem cells according to any of the methods herein disclosed.

An embodiment of the invention will know be described by example only and with reference to the following figures:

FIG. 1 is a schematic represenation of conserved domains in Notch polypeptides;

FIG. 2 is the nucleic acid sequence of murine notch ligand delta-like 1;

FIG. 3 is the amino acid sequence of murine notch ligand delta-like 1;

FIG. 4 is the nucleic acid sequence of murine notch ligand jagged 1;

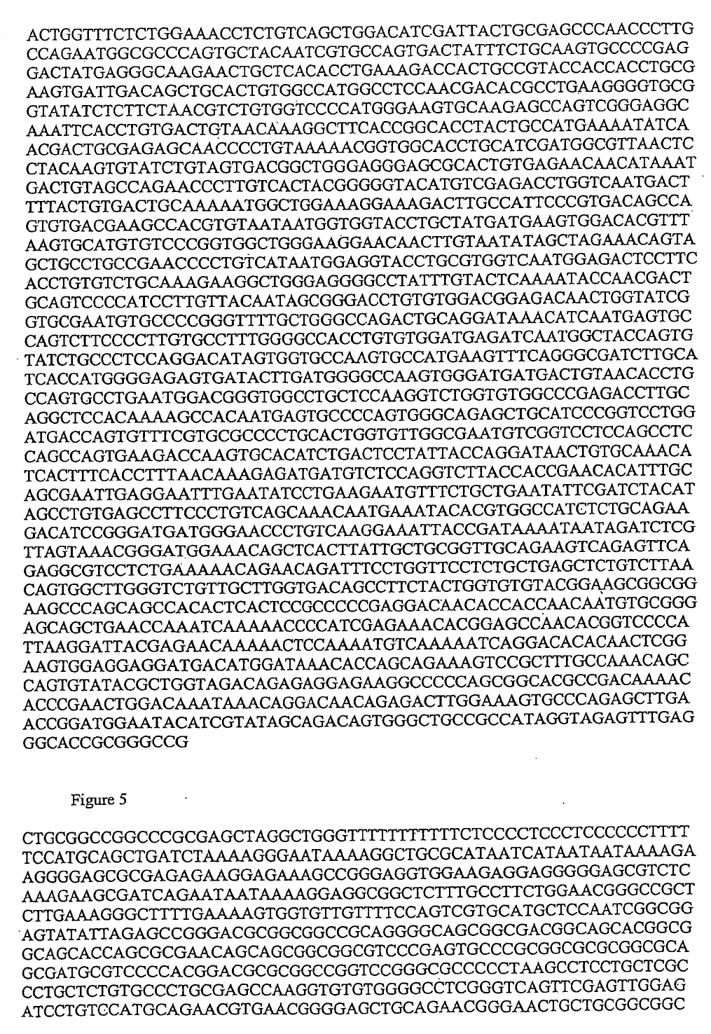

FIG. 5 is the nucleic acid sequence of human notch ligand jagged 1 (alagille syndrome) (JAG1);

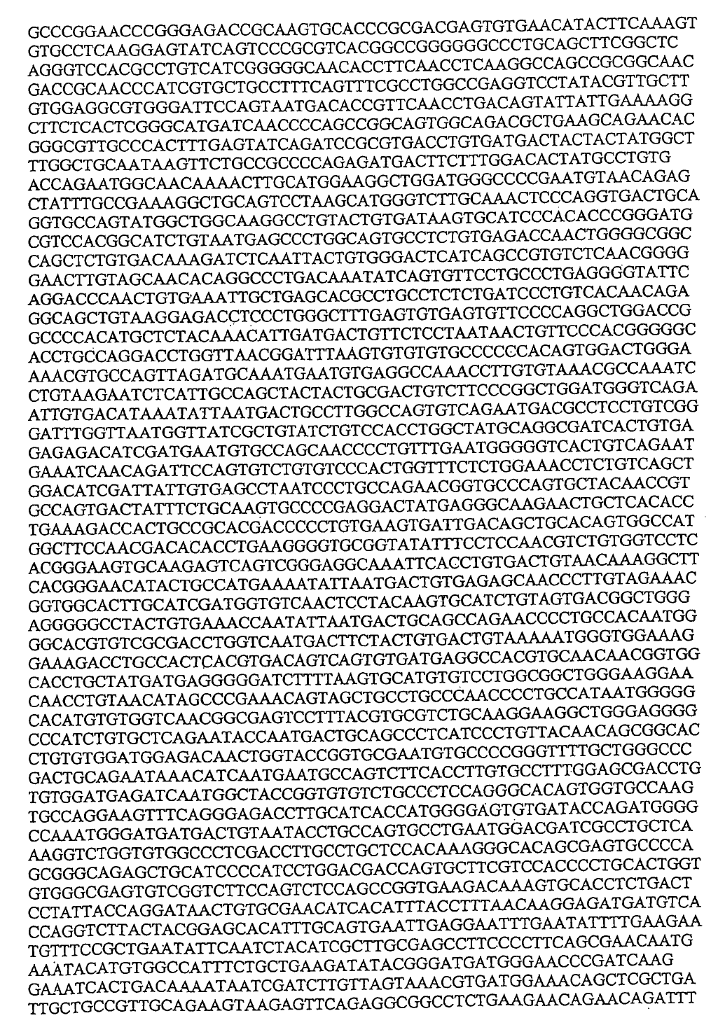

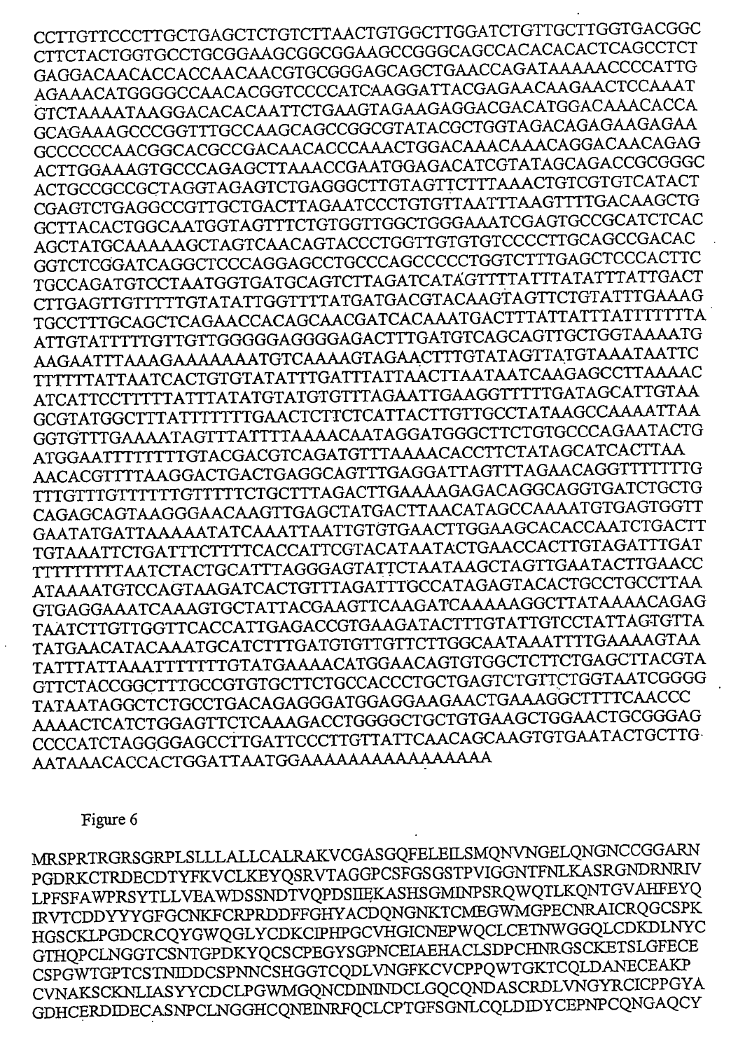

FIG. 6 is the amino acid sequence of human notch ligand jagged 1 (alagille syndrome);

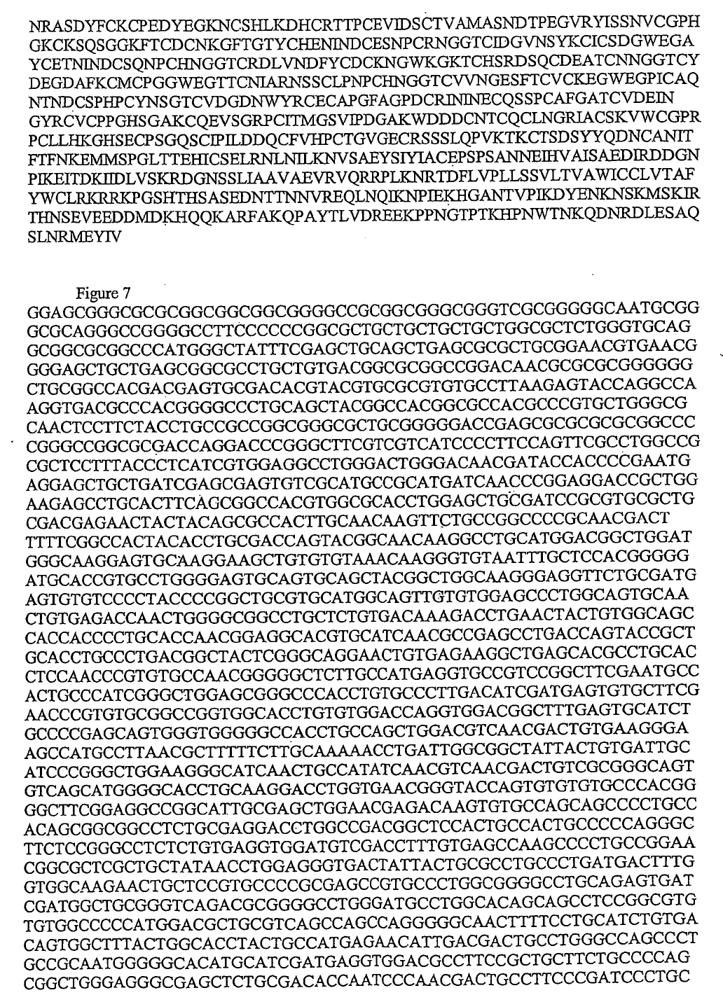

FIG. 7 is the nucleic acid sequence of human notch ligand jagged 2 (JAG2)

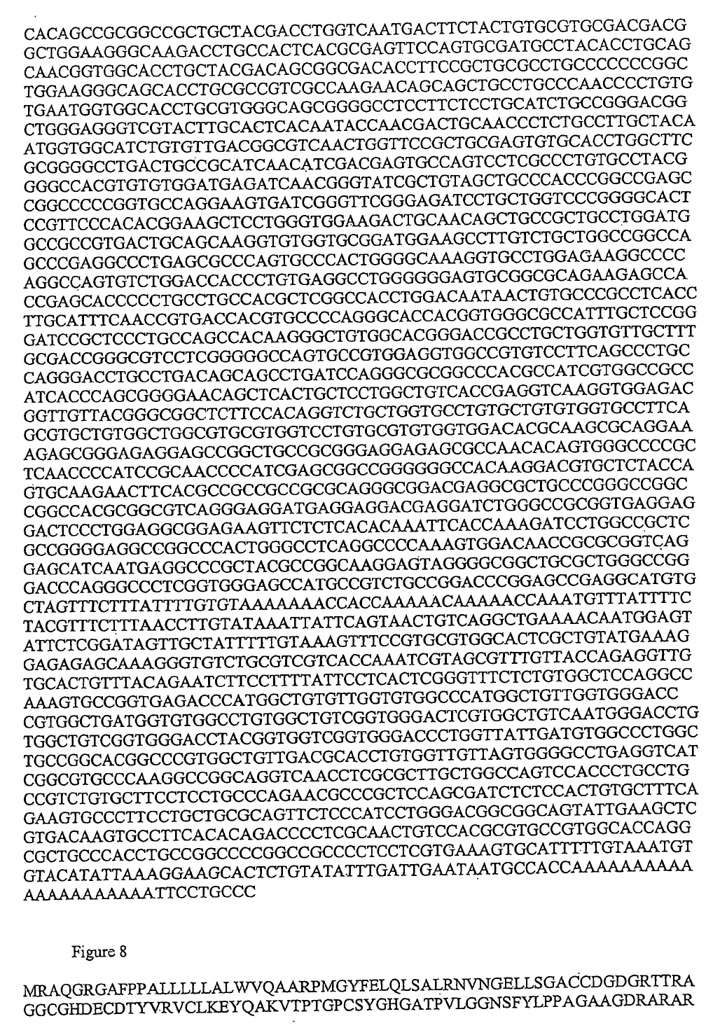

FIG. 8 is the amino acid sequence of human notch ligand jagged 2 (JAG2);

FIG. 22 represents the nucleic acid sequence of human Wnt 13;

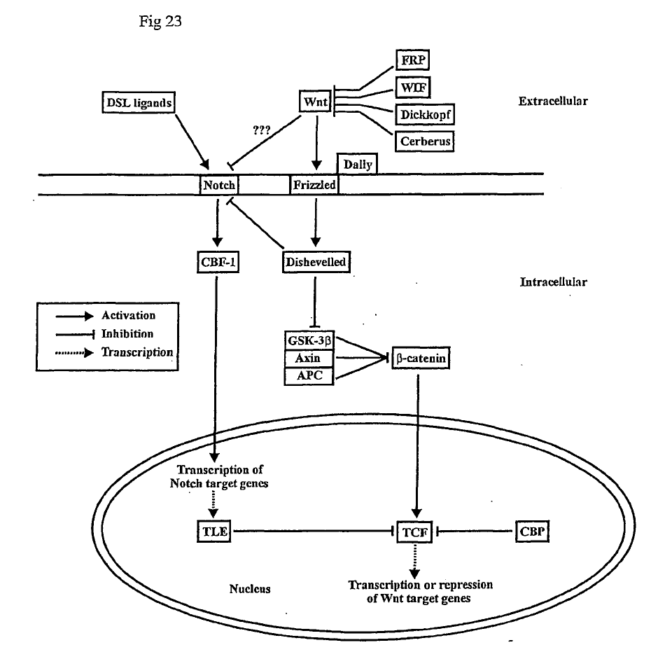

FIG. 23 is a diagramatic representation of the Wnt signalling pathway,

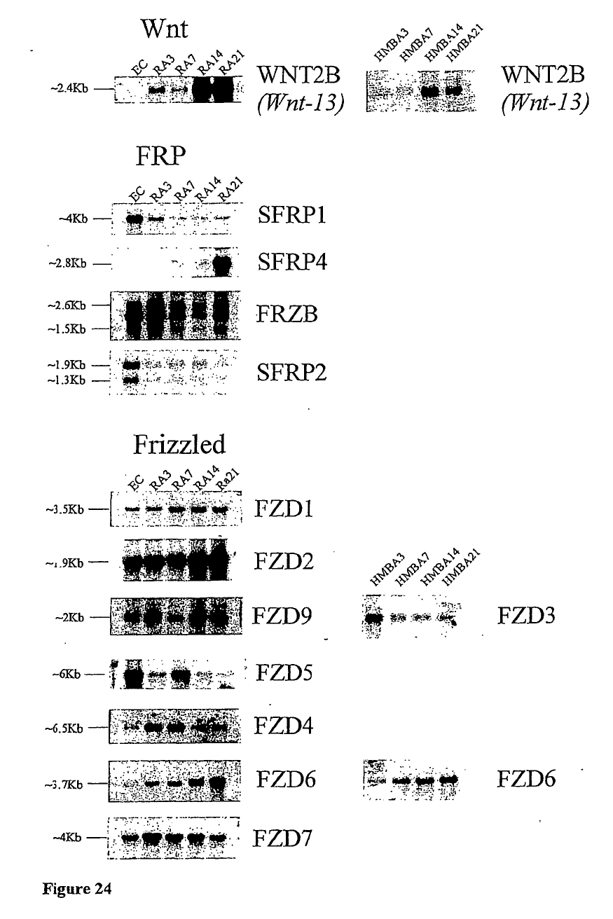

FIG. 24 represents northern blot analysis of Wnt 13 and mrRNA's corresponding to Frizzled receptors and Frizzled related protein antagonists of Wnt signalling in NTERA 2 cells various Wnt inhibitors after exposure of NTERA 2 cells;

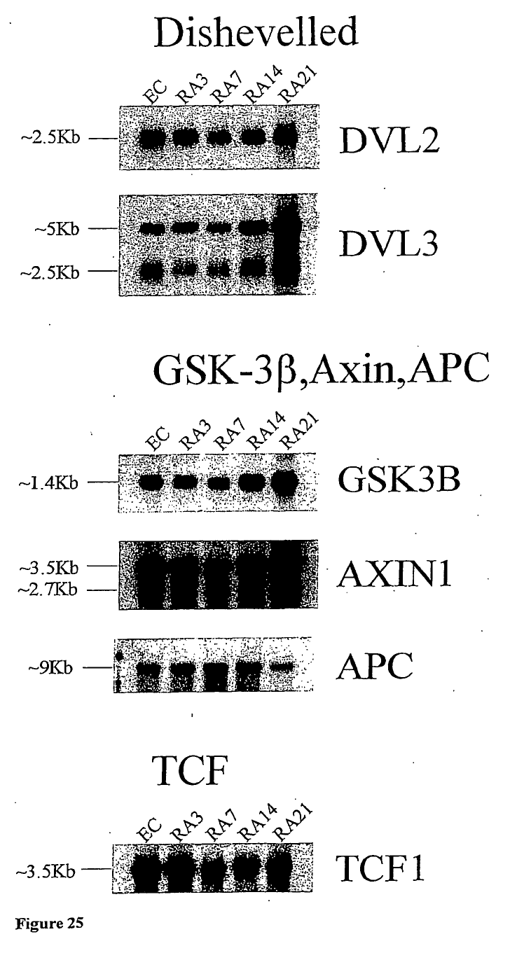

FIG. 25 represents a northern blot analysis of intracellular components of Wnt signalling pathway in NTERA 2 cells;

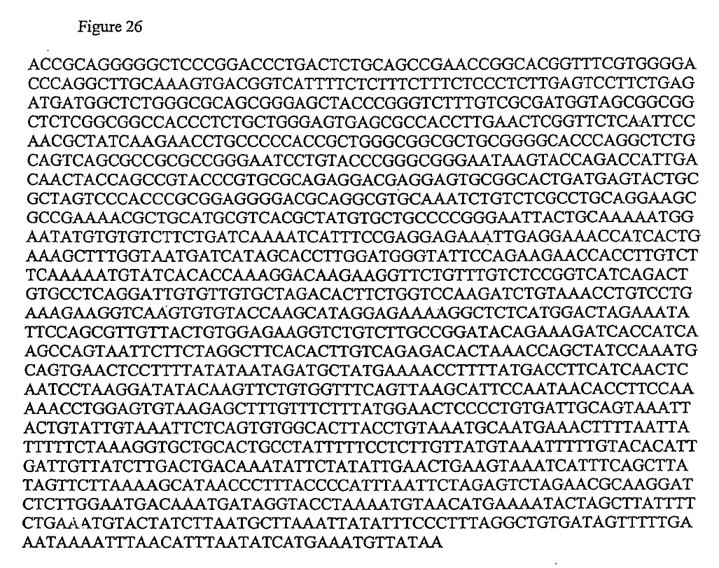

FIG. 26 represents the nucleic acid sequence of human dickkopf1;

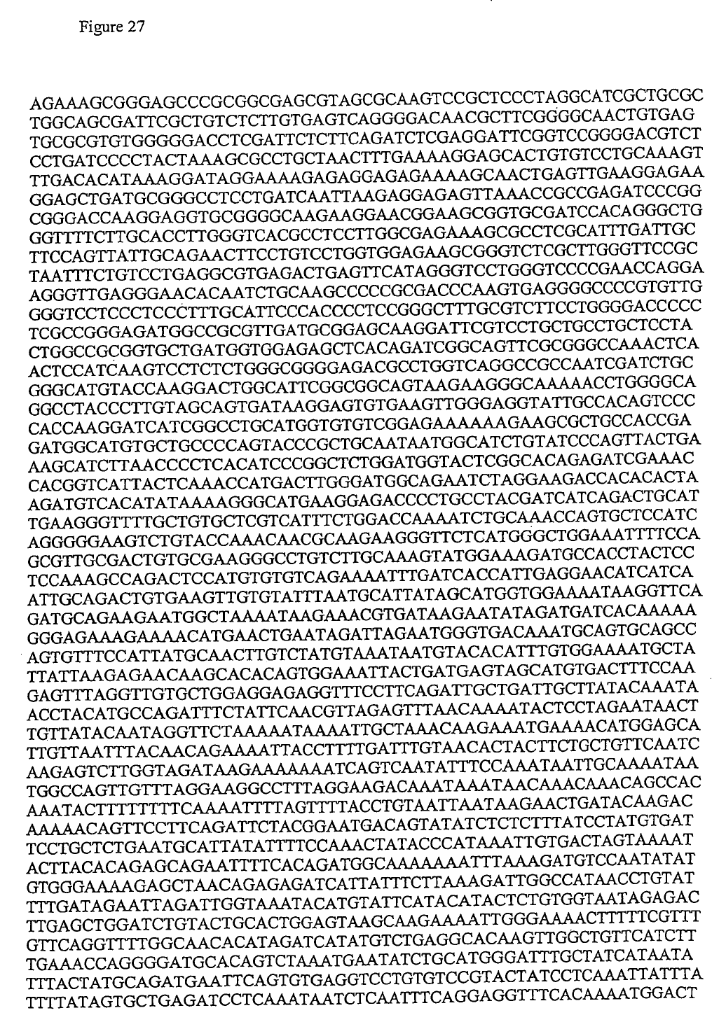

FIG. 27 represents the nucleic acid sequence of human dickkopf2;

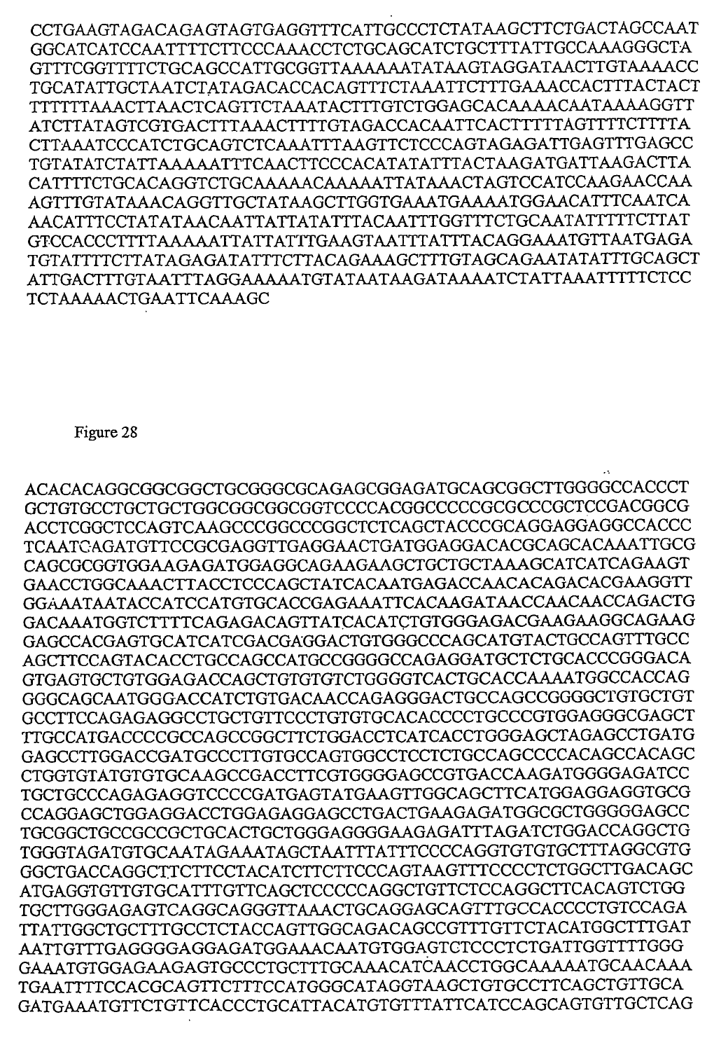

FIG. 28 represents the nucleic acid sequence of human dickkopf3 and

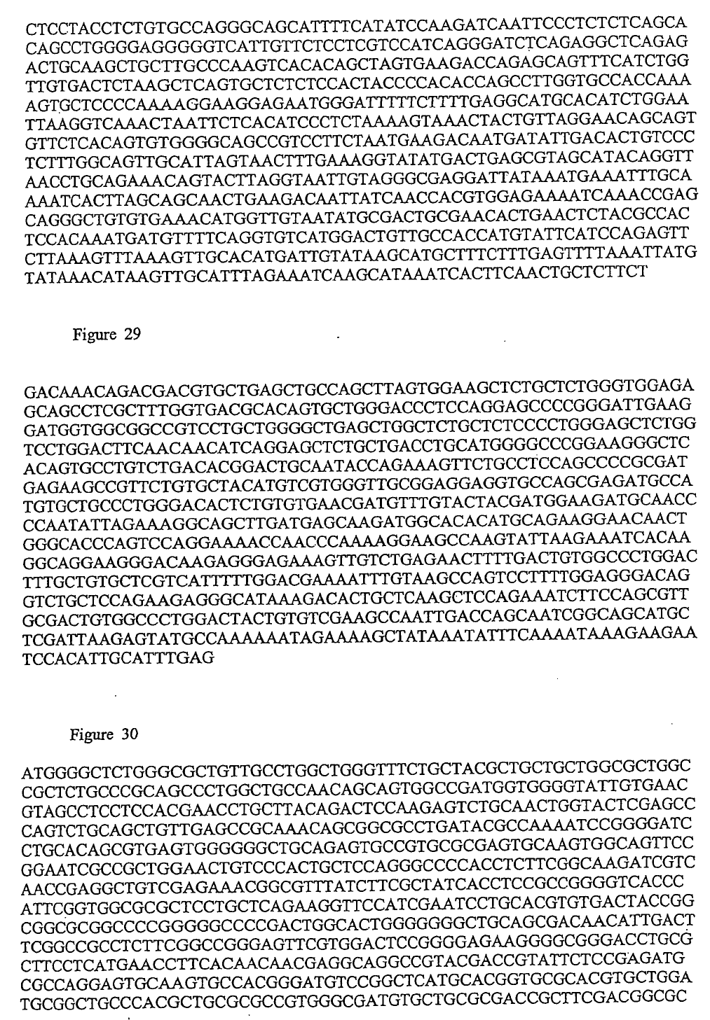

FIG. 29 represents the nucleic acid sequence of human dickkopf4;

FIG. 30 represents the nucleic acid sequence of WNT-1;

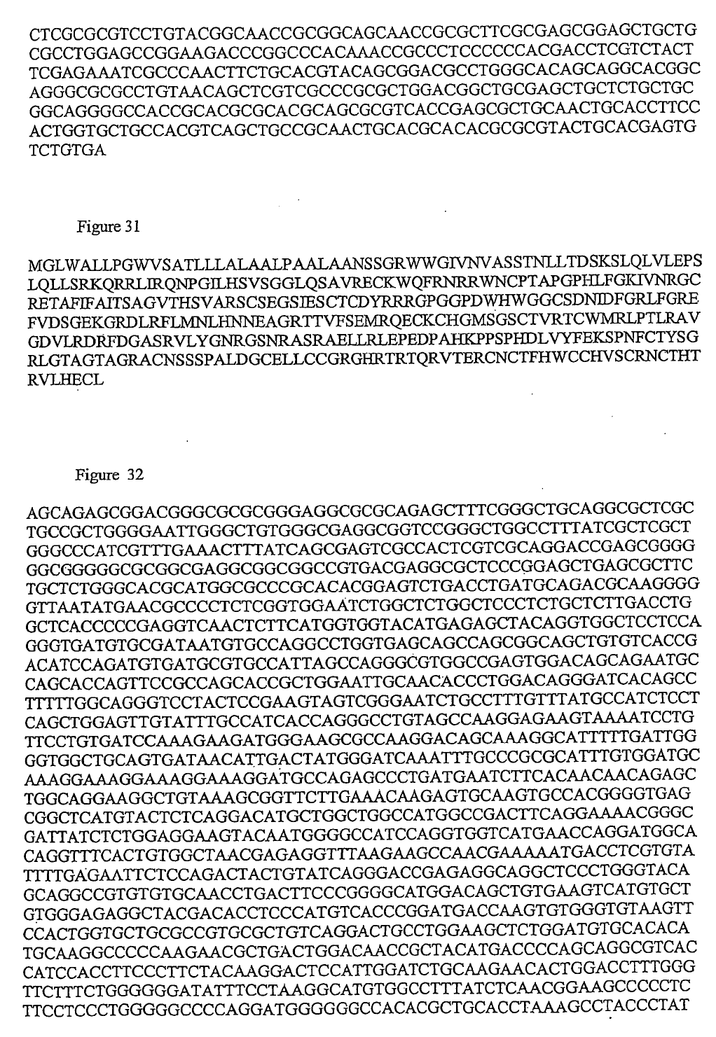

FIG. 31 represents the amino acid sequence of WNT-1;

FIG. 32 represents the nucleic acid sequence of WNT-2;

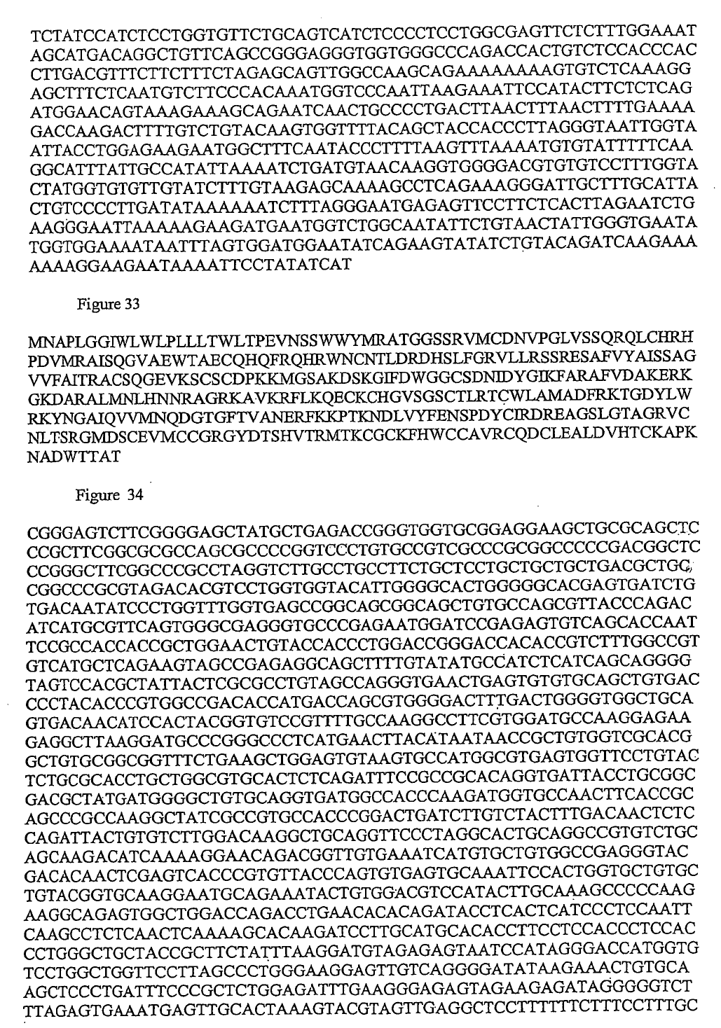

FIG. 33 represents the amino acid sequence of WNT-2;

FIG. 34 represents the nucleic acid sequence of WNT 2B;

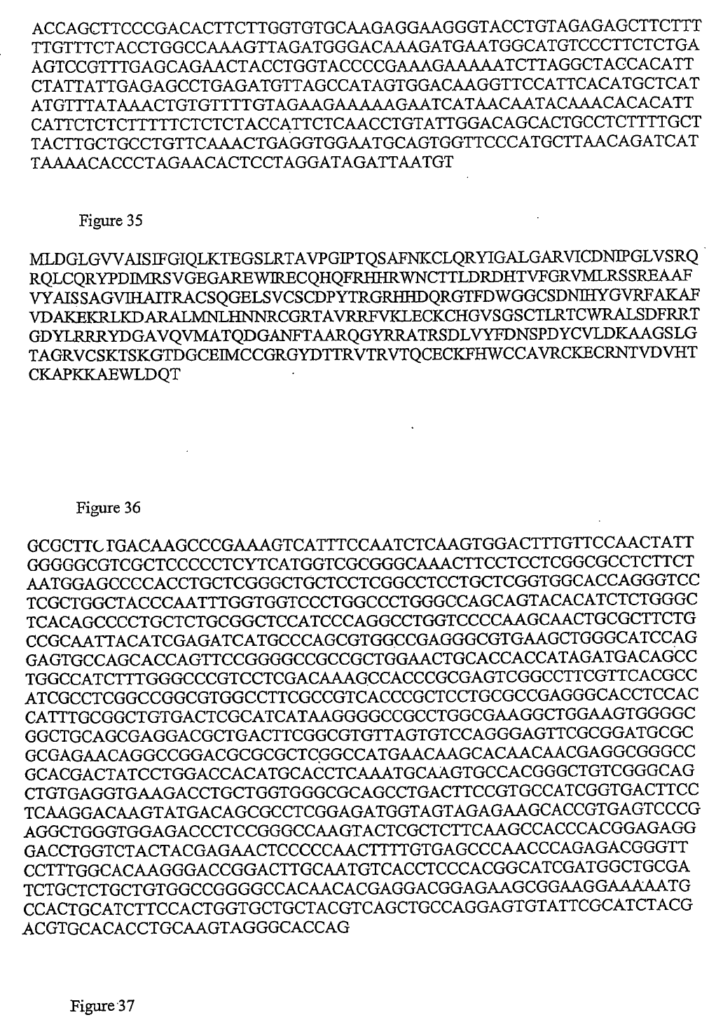

FIG. 35 represents the amino acid sequence of WNT 2B;

FIG. 36 represents the nucleic acid sequence of WNT 3;

FIG. 37 represents the amino acid sequence of WNT 3;

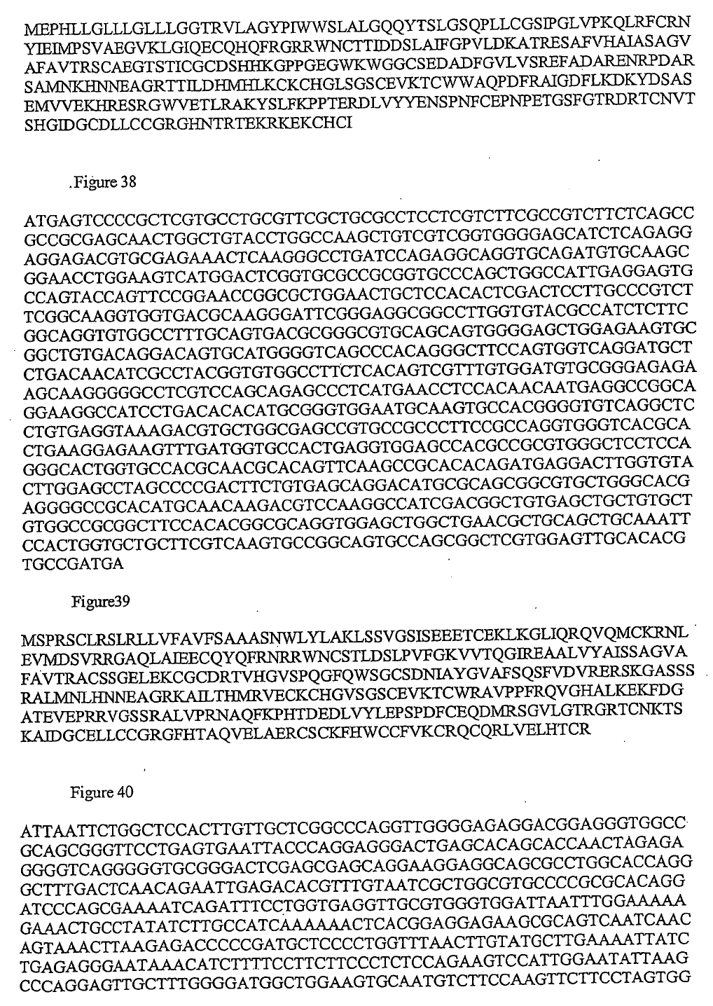

FIG. 38 represents the nucleic acid sequence of WNT 4;

FIG. 39 represents the amino acid sequence of WNT 4;

FIG. 40 represents the nucleic acid sequence of WNT 5A;

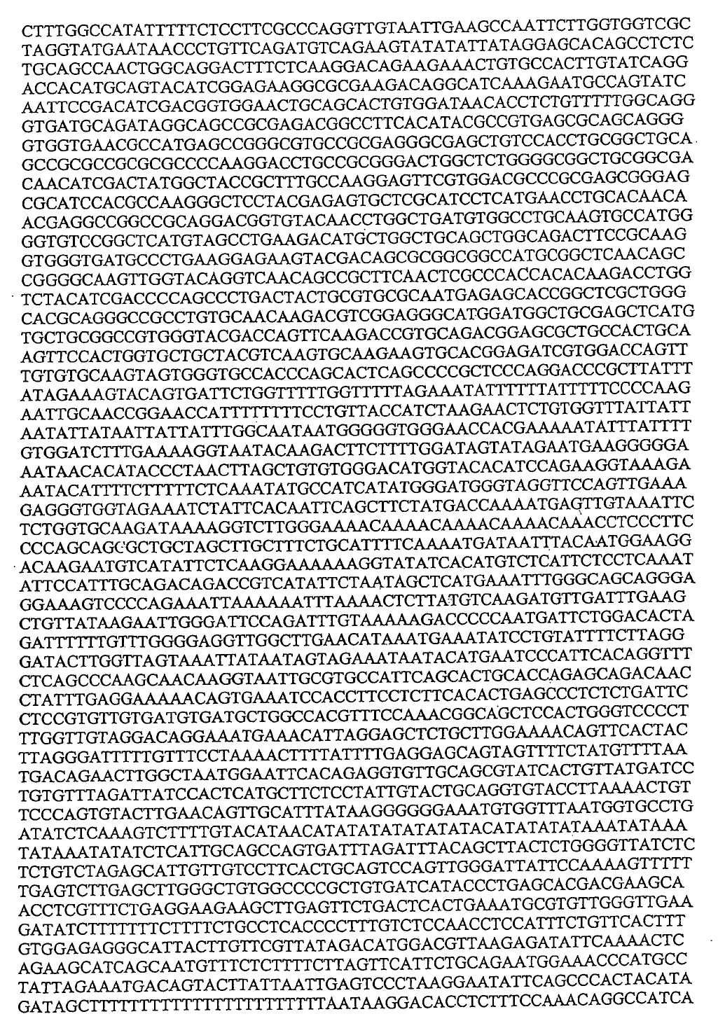

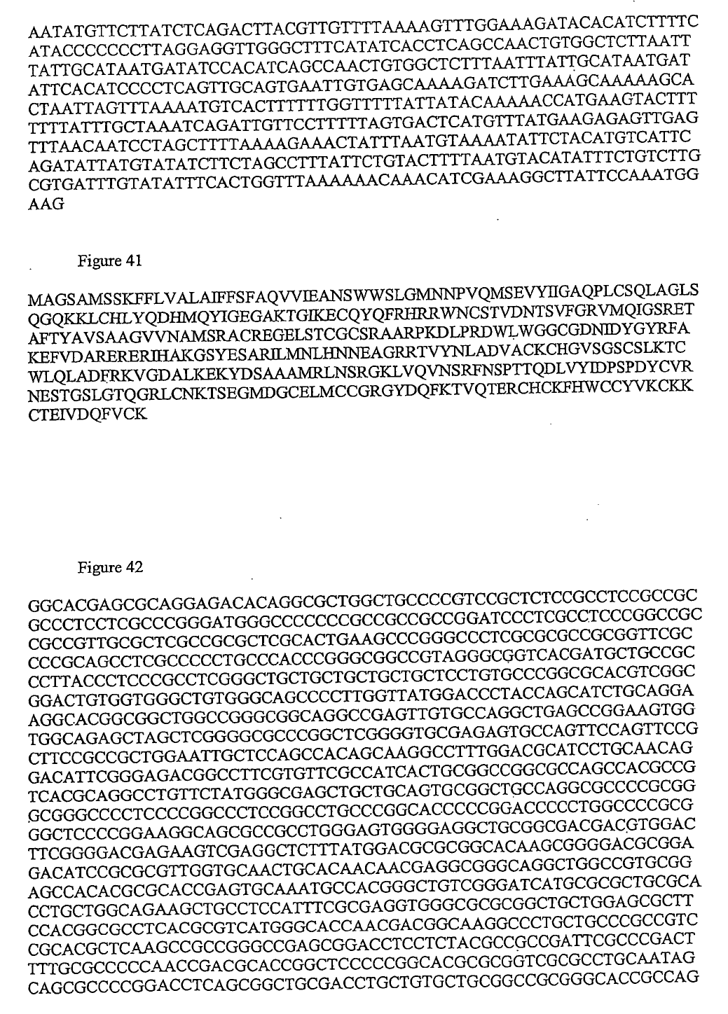

FIG. 41 represents the amino acid sequence of WNT 5A;

FIG. 42 represents the nucleic acid sequence of WNT 6;

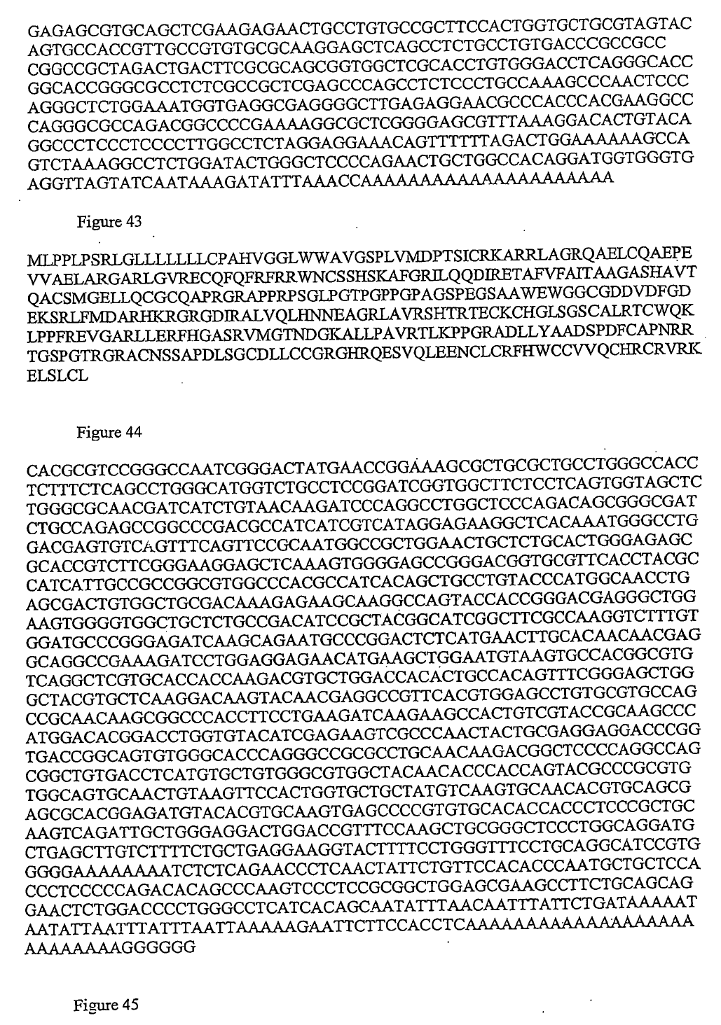

FIG. 43 represents the amino acid sequence of WNT 6;

FIG. 44 represents the nucleic acid sequence of WNT 7A;

FIG. 45 represents the amino acid sequence of WNT 7A;

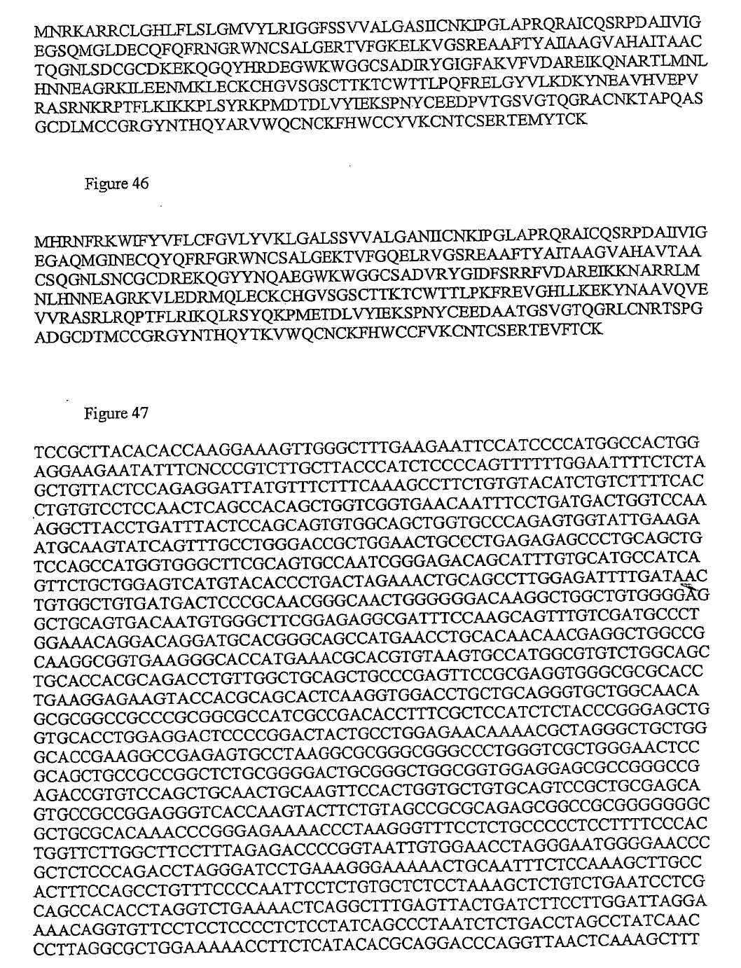

FIG. 46 represents the amino acid sequence of WNT 7B;

FIG. 47 represents the nucleic acid sequence of WNT 8B;

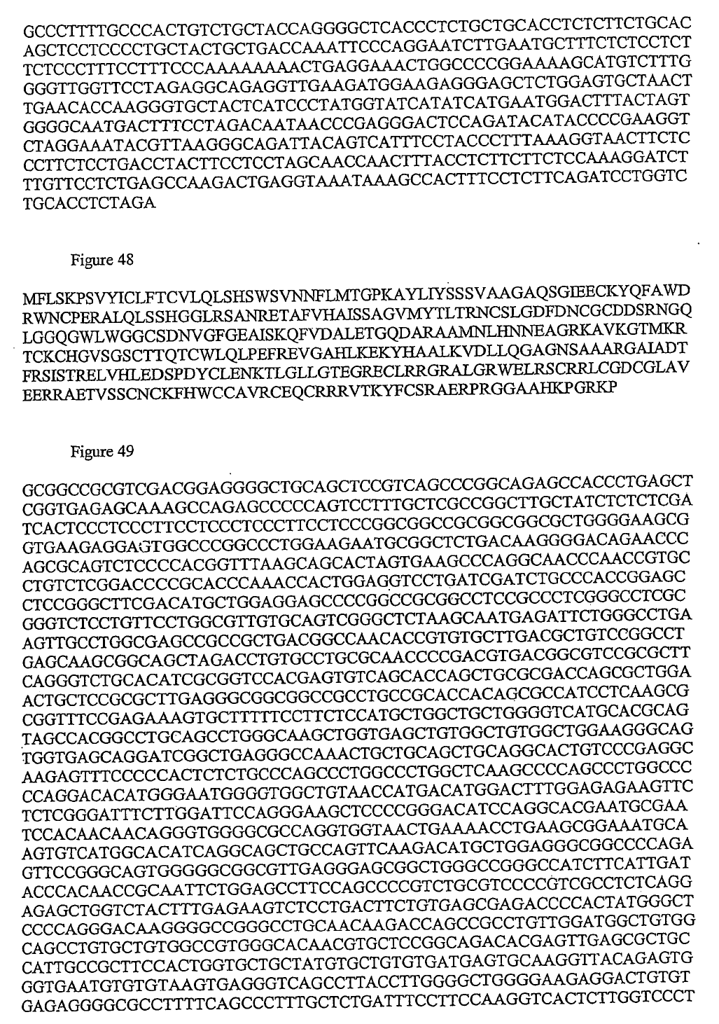

FIG. 48 represents the amino acid sequence of WNT 8B;

FIG. 49 represents the nucleic acid sequence of WNT 10B;

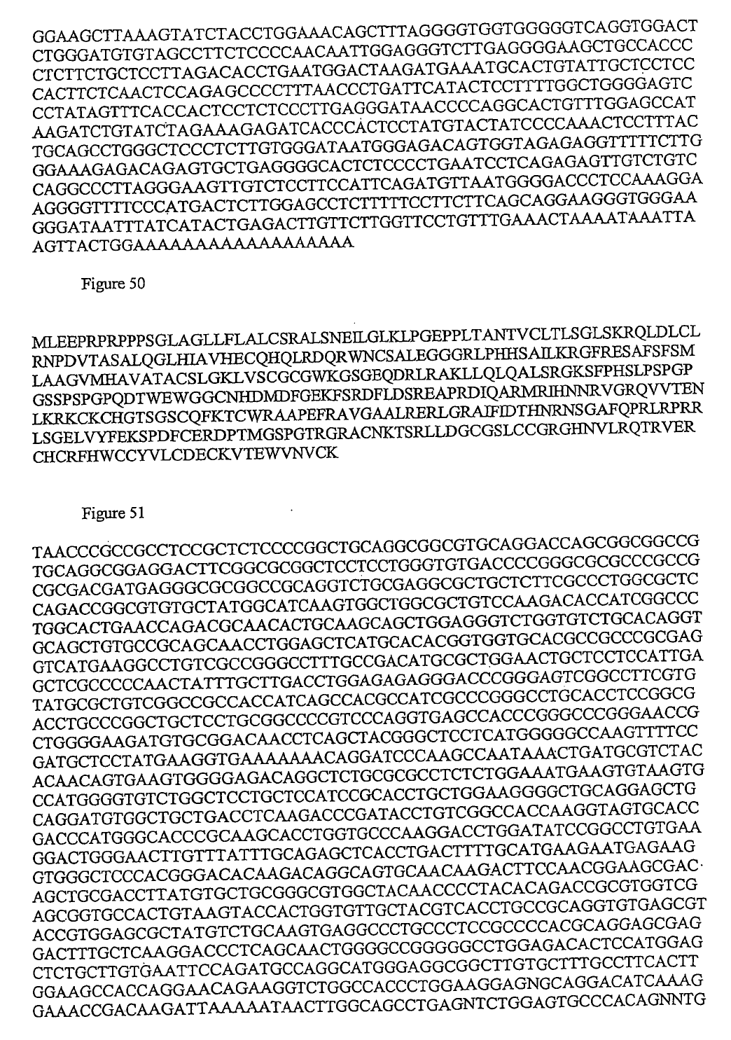

FIG. 50 represents the amino acid sequence of WNT 10B;

FIG. 51 represents the nucleic acid sequence of WNT 11;

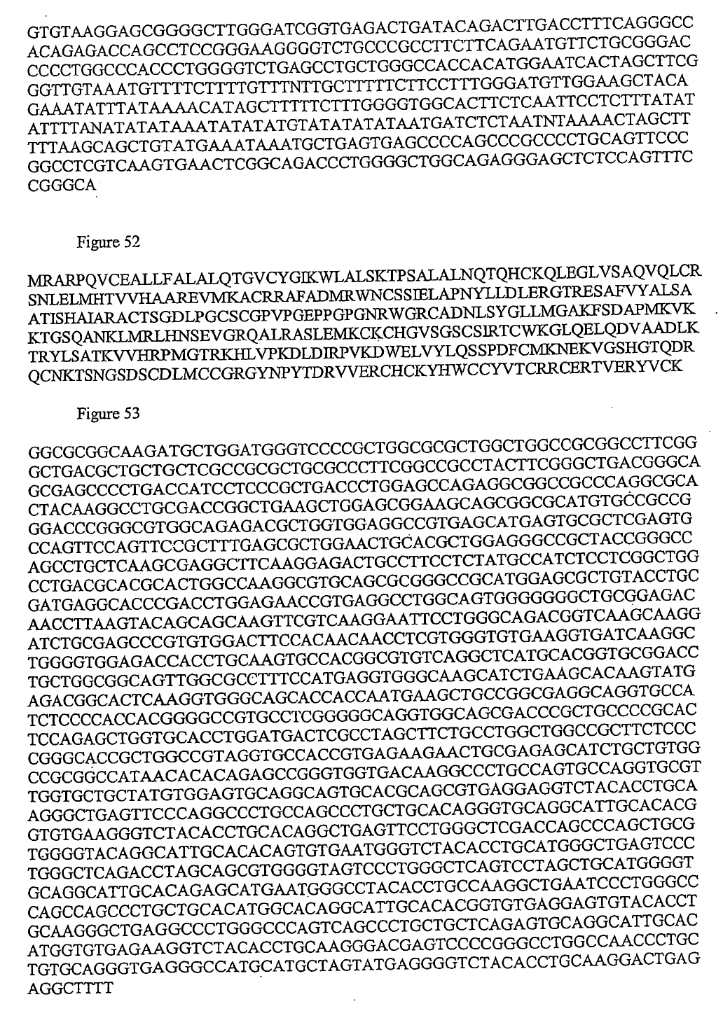

FIG. 52 represents the amino acid sequence of WNT 11;

FIG. 53 represents the nucleic acid sequence of WNT 14

FIG. 54 represents the amino acid sequence of WNT 14;

FIG. 55 represents the nucleic acid sequence of WNT 16;

FIG. 56 represents the amino acid sequence of WNT 16;

FIG. 57 represents the nucleic acid sequence of FZD 1;

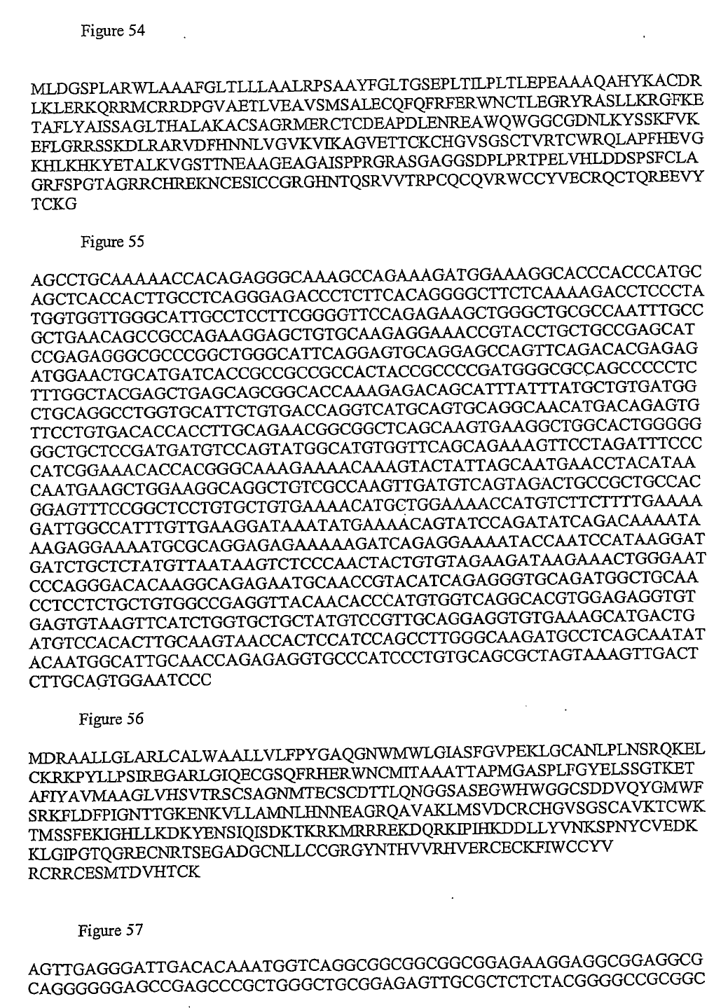

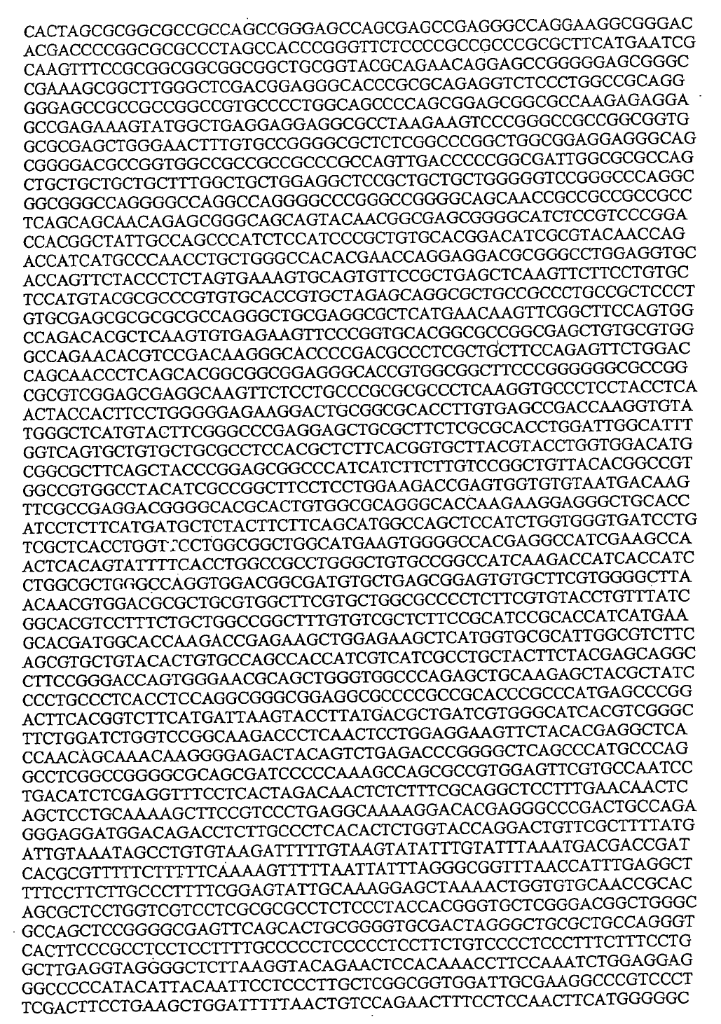

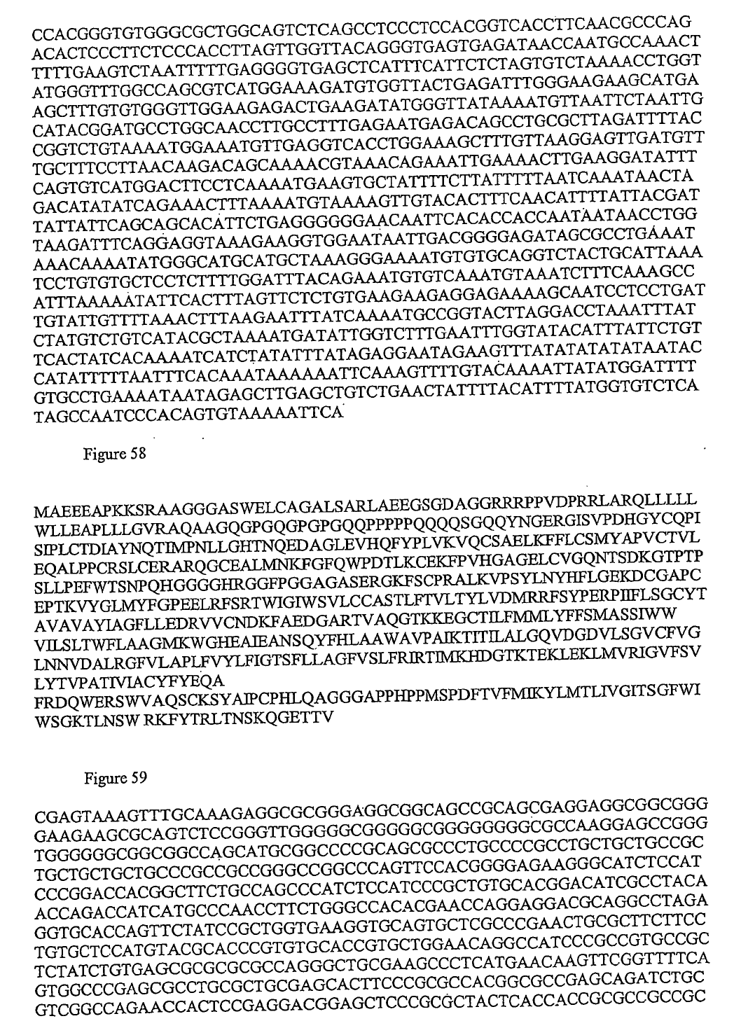

FIG. 58 represents the amino acid sequence of FZD 1;

FIG. 59 represents the nucleic acid sequence of FZD 2;

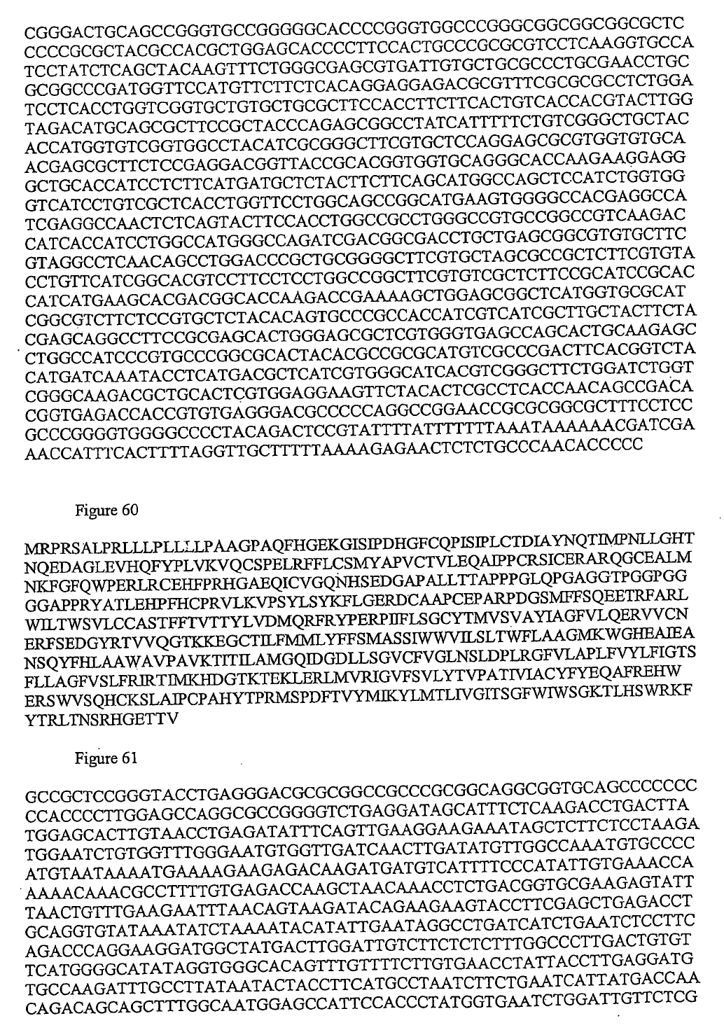

FIG. 60 represents the amino acid sequence of FZD 2;

FIG. 61 represents the nucleic acid sequence of FZE 3;

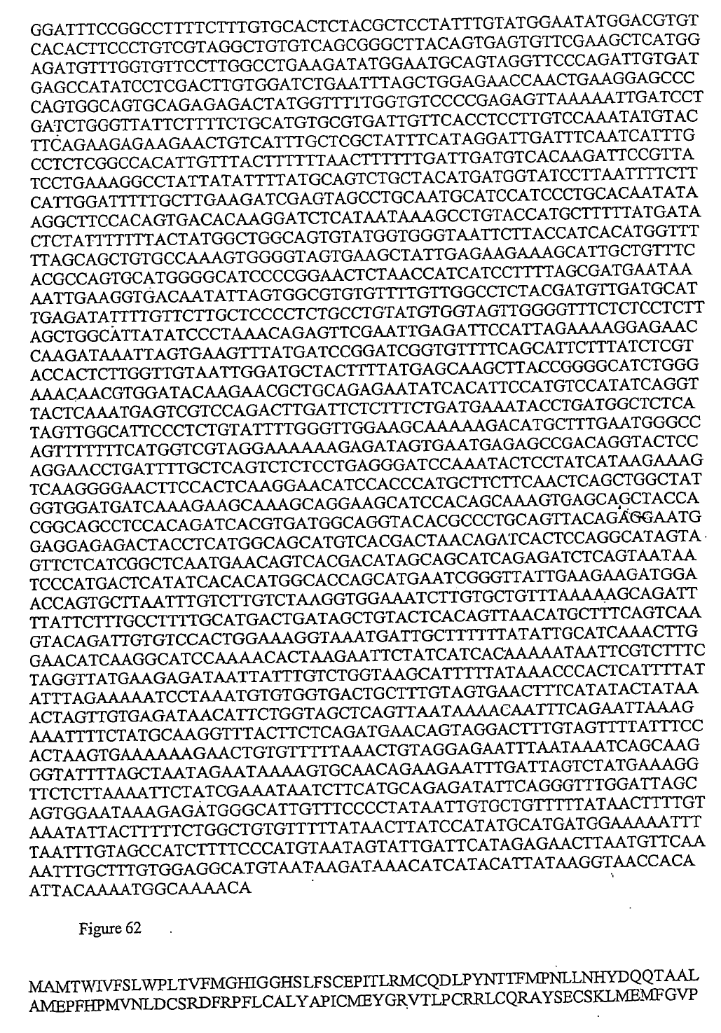

FIG. 62 represents the amino acid sequence of FZE 3;

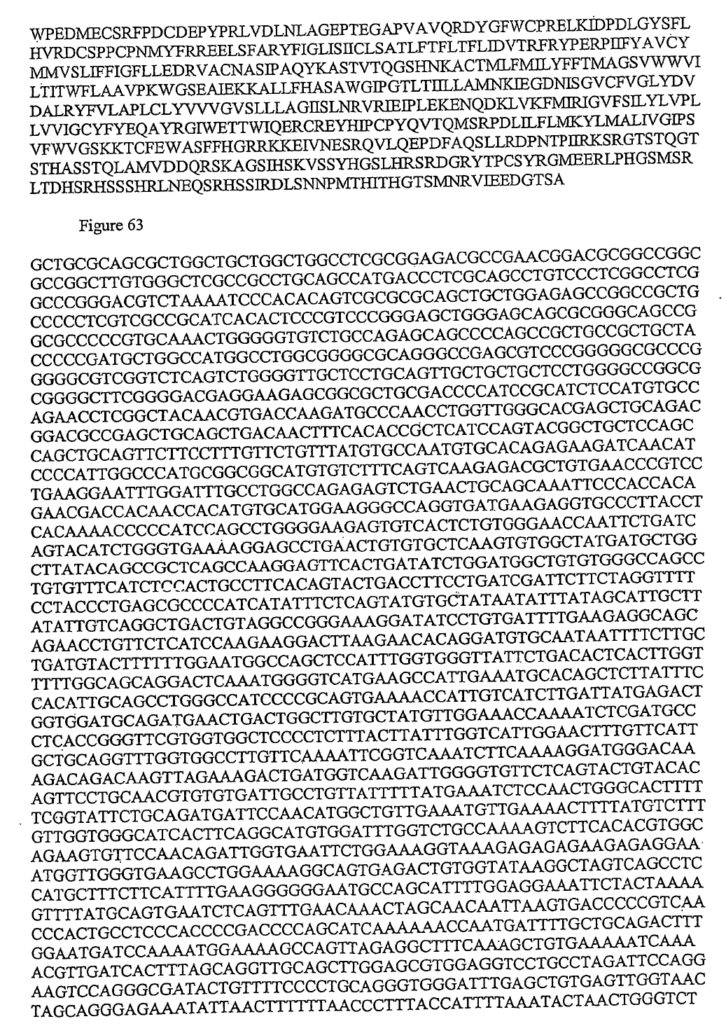

FIG. 63 represents the nucleic acid sequence of FZD 4;

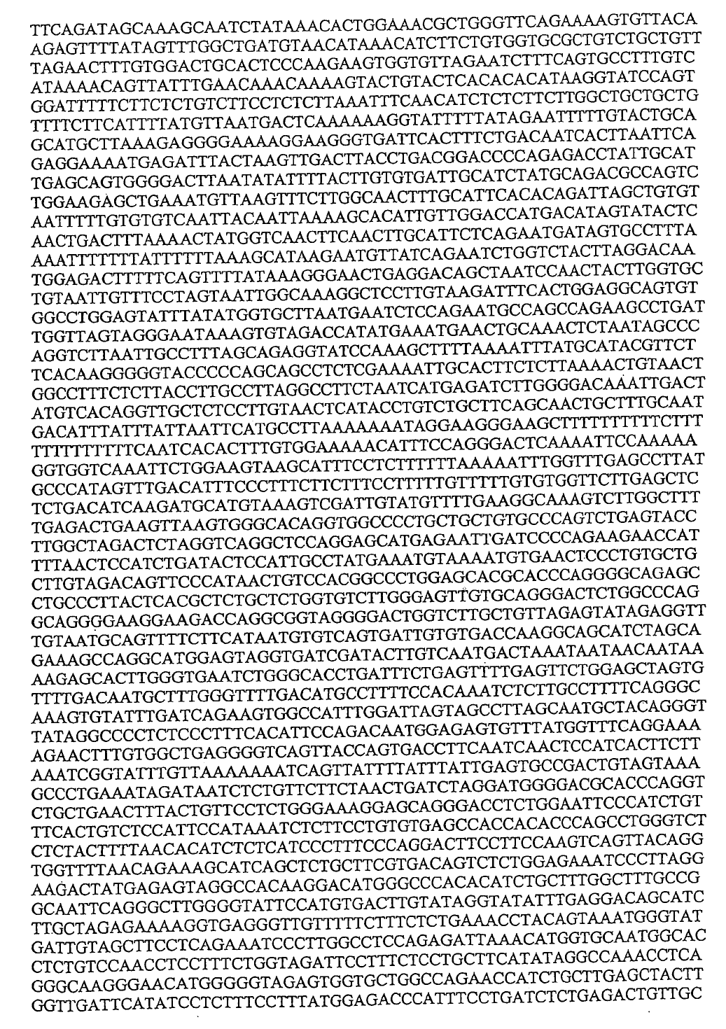

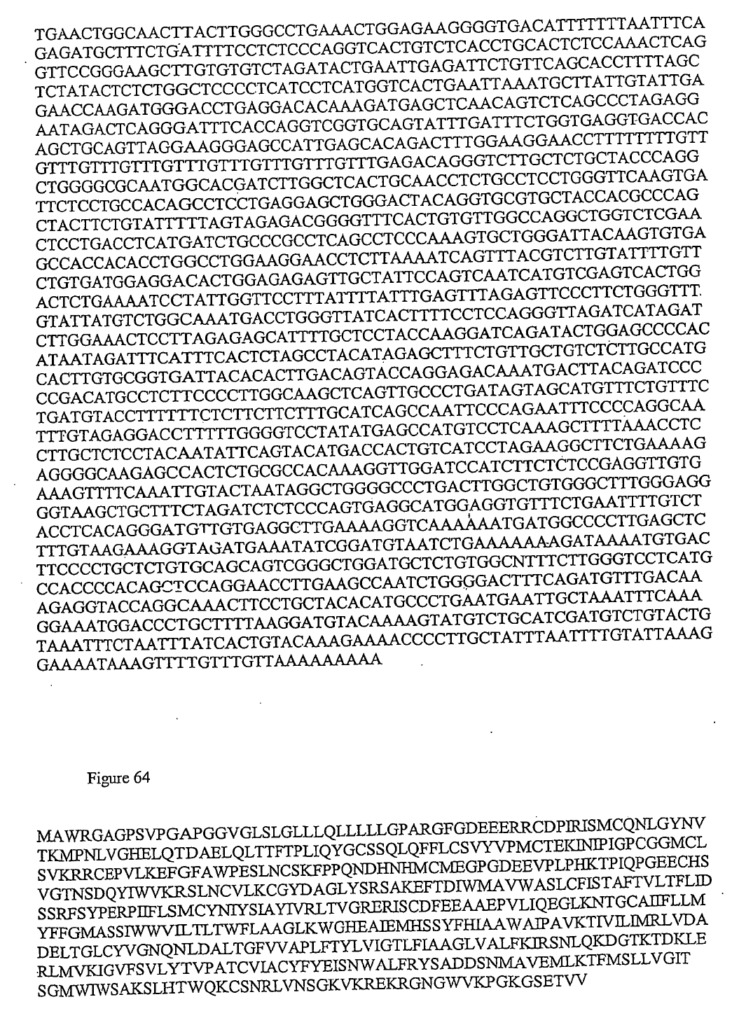

FIG. 64 represents the amino acid sequence of FZD 4;

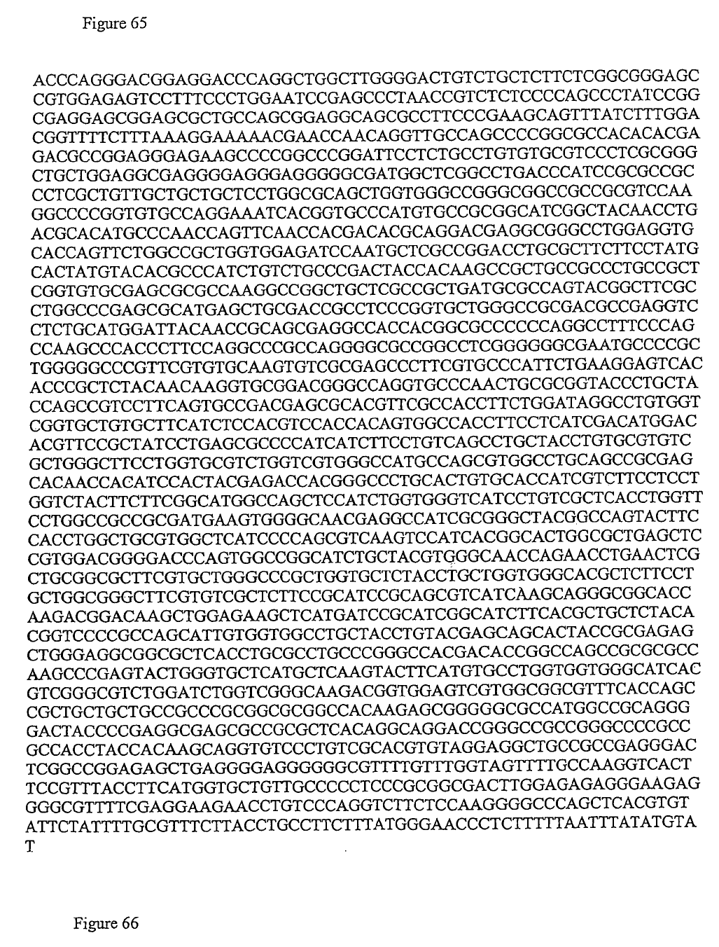

FIG. 65 represents the nucleic acid sequence of FZD 5;

FIG. 66 represents the amino acid sequence of FZD 5;

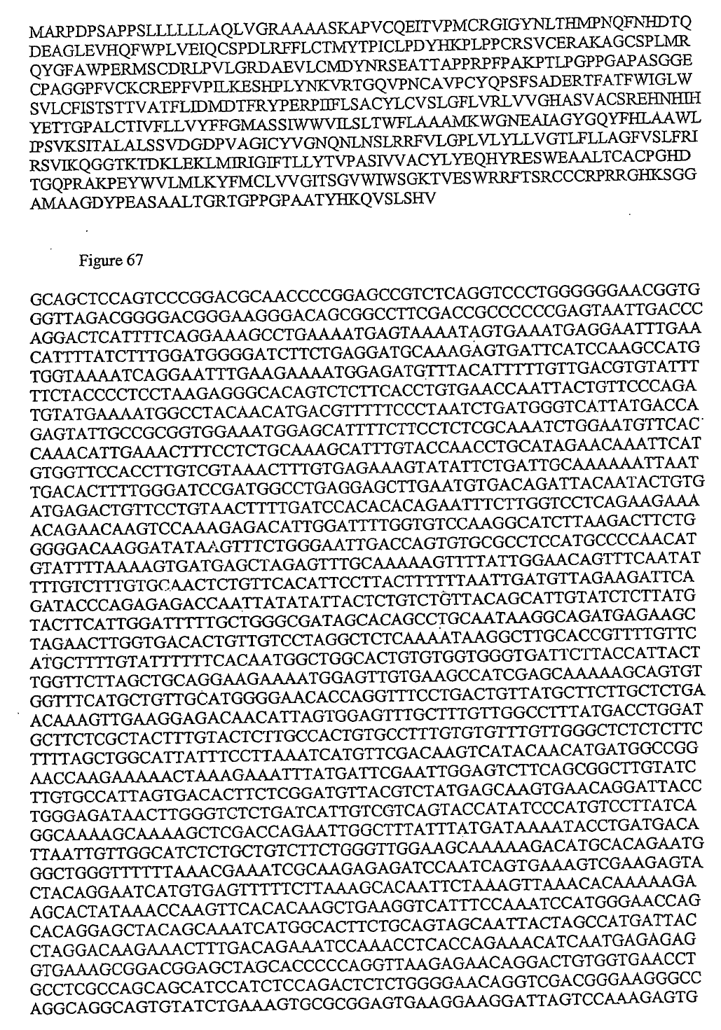

FIG. 67 represents the nucleic acid sequence of FZD 6;

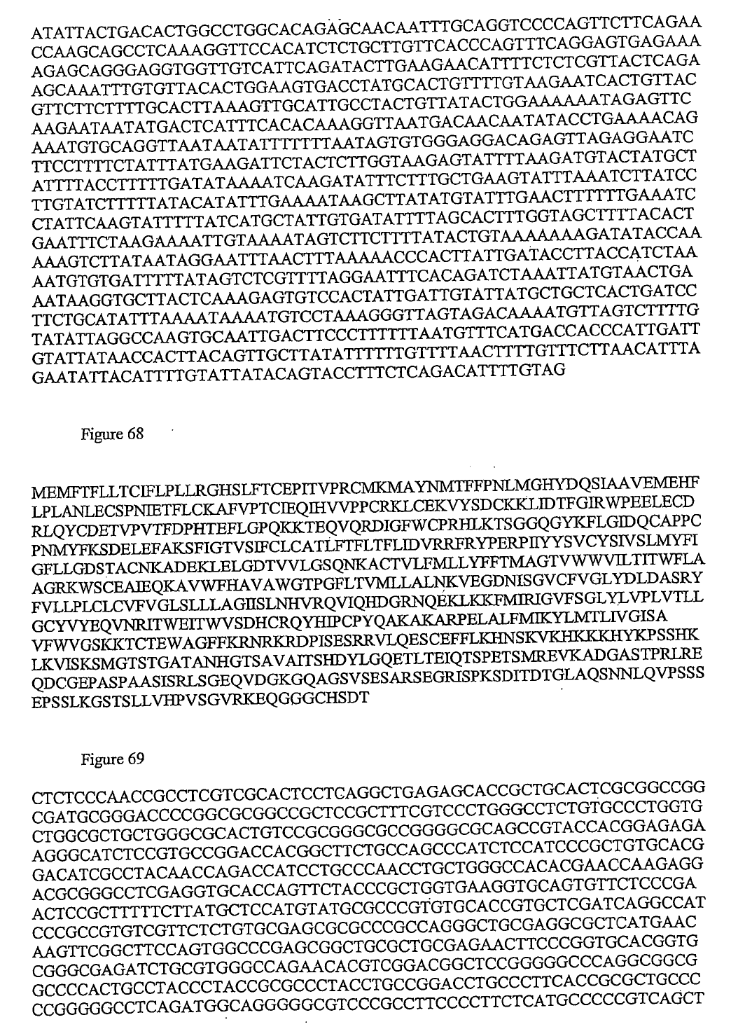

FIG. 68 represents the amino acid sequence of FZD 6;

FIG. 69 represents the nucleic acid sequence of FZD 7;

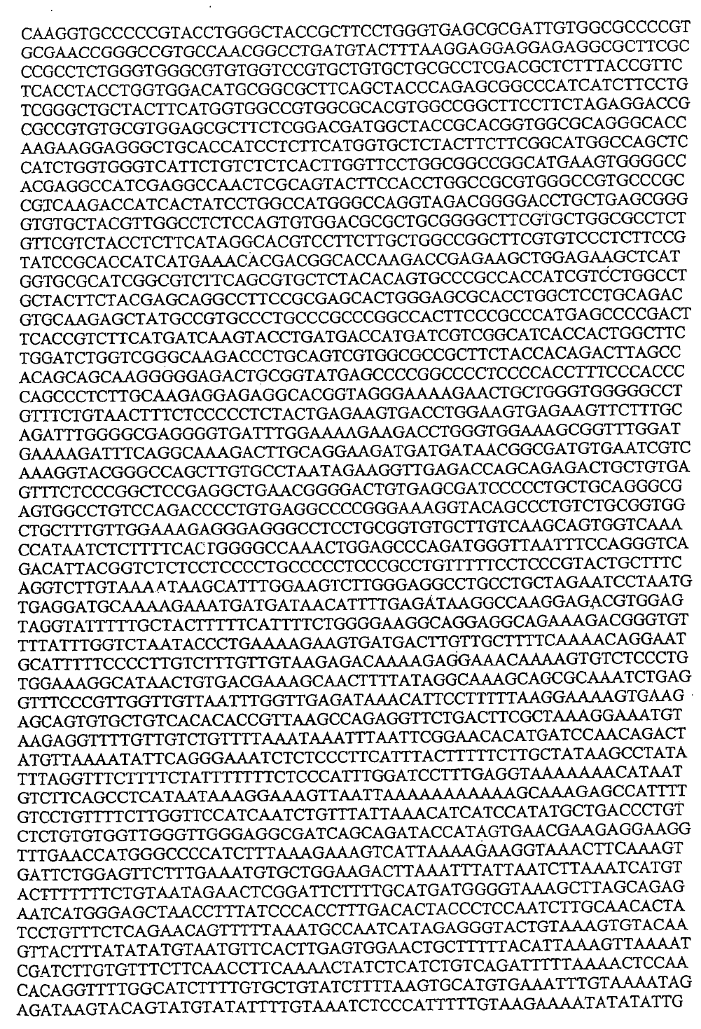

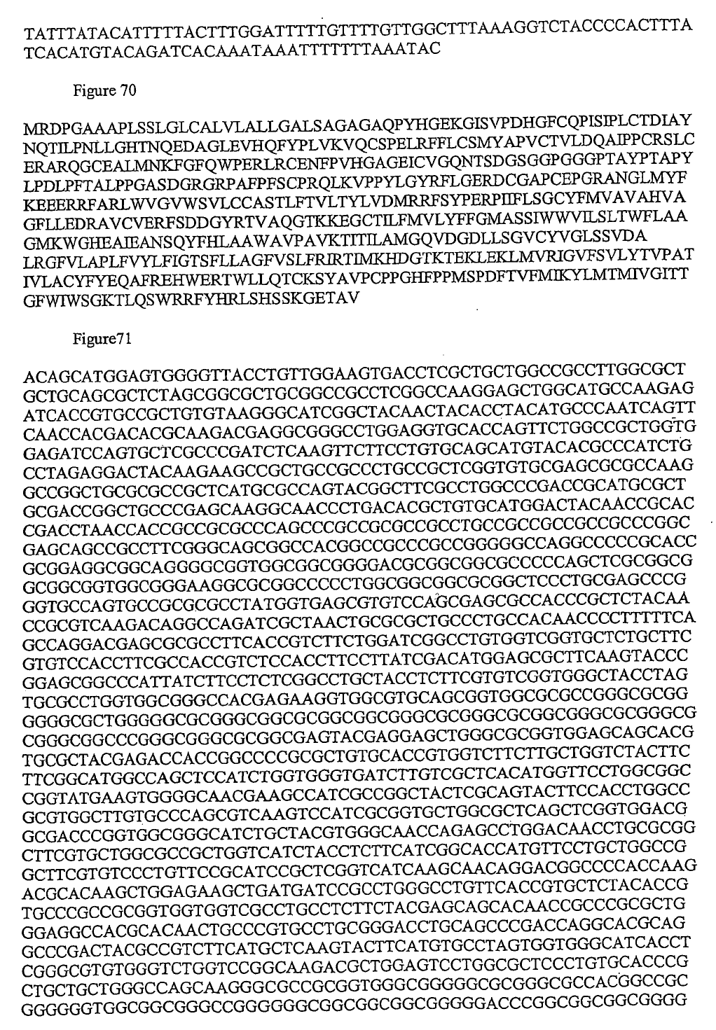

FIG. 70 represents the amino acid sequence of FZD 7;

FIG. 71 represents the nucleic acid sequence of FZD 8;

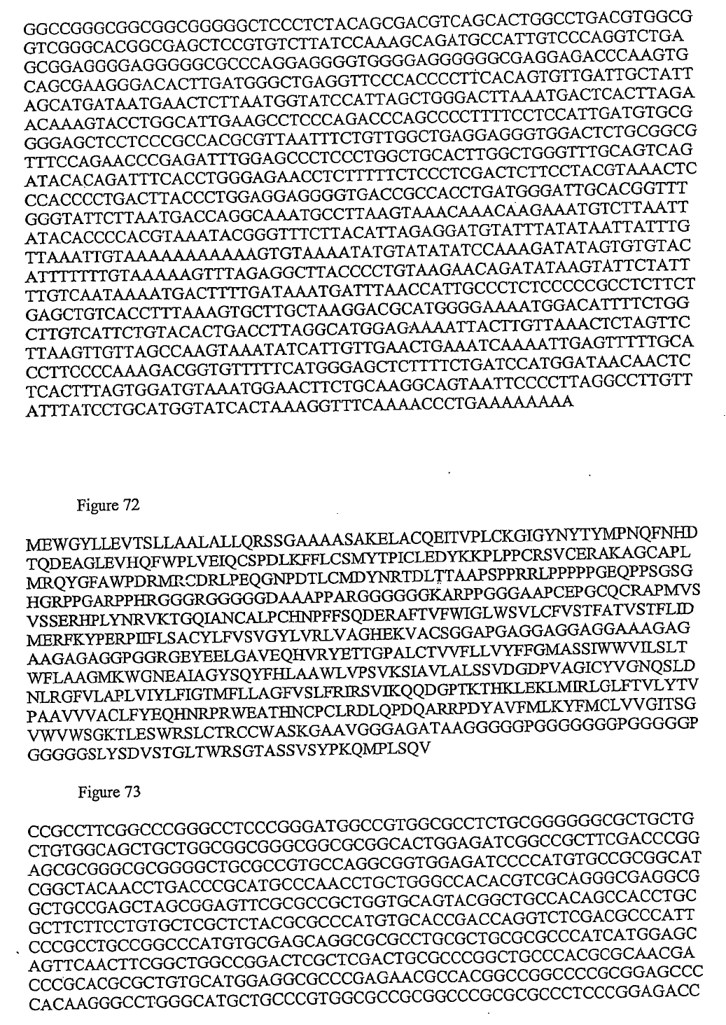

FIG. 72 represents the amino acid sequence of FZD 8;

FIG. 73 represents the nucleic acid sequence of FZD 9;

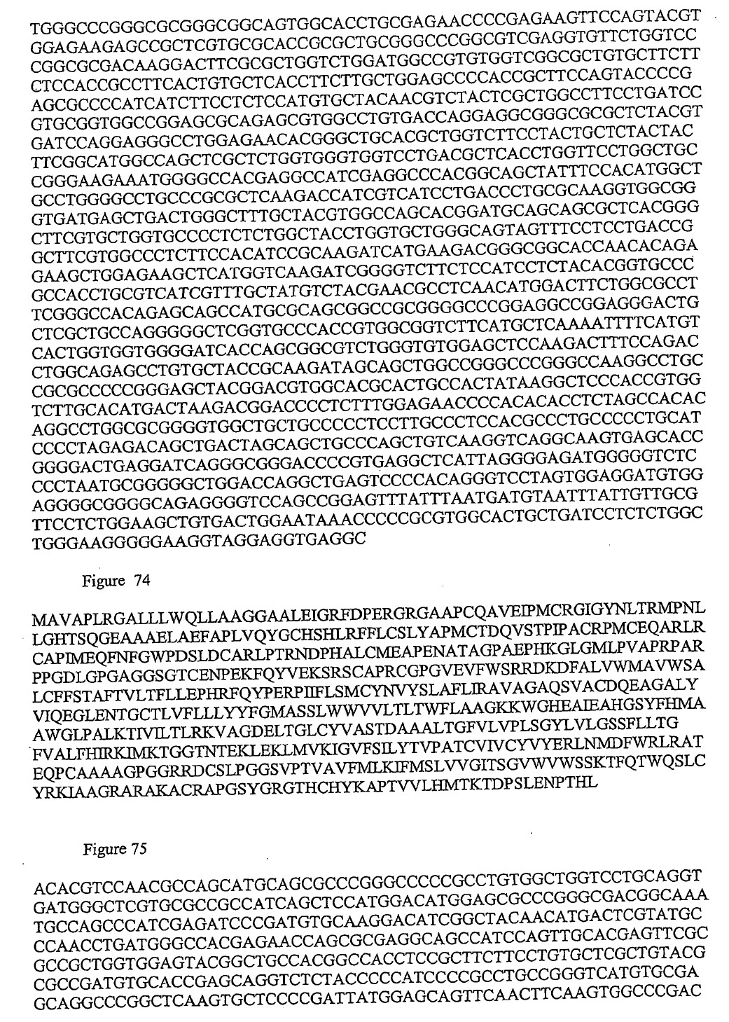

FIG. 74 represents the amino acid sequence of FZD 9;

FIG. 75 represents the nucleic acid sequence of FZD 10;

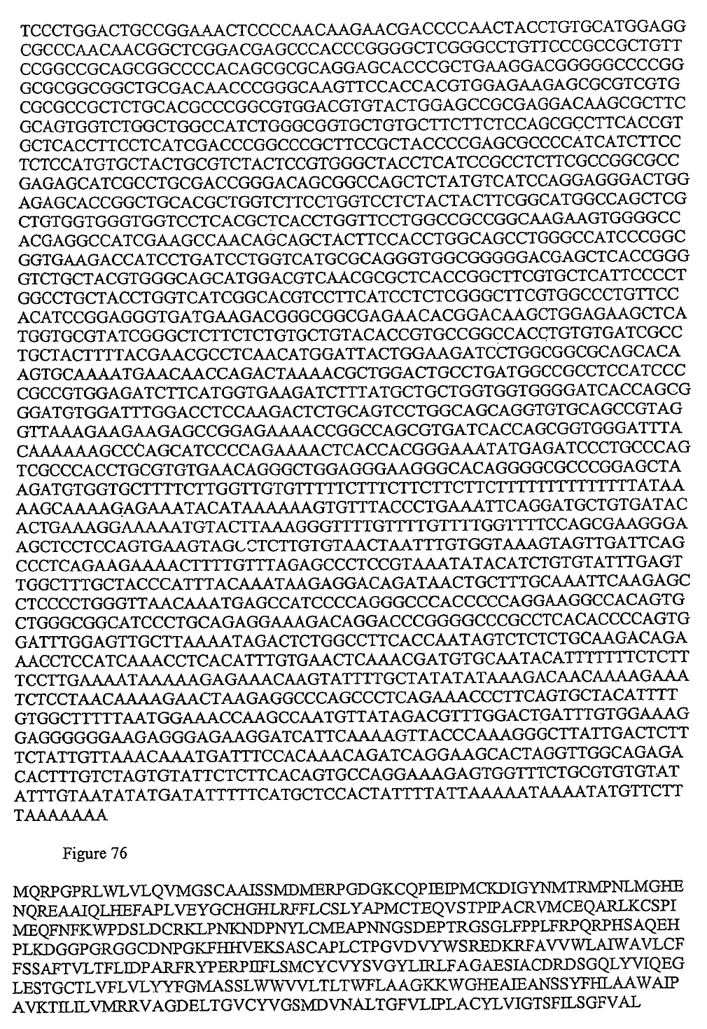

FIG. 76 represents the amino acid sequence of FZD 10;

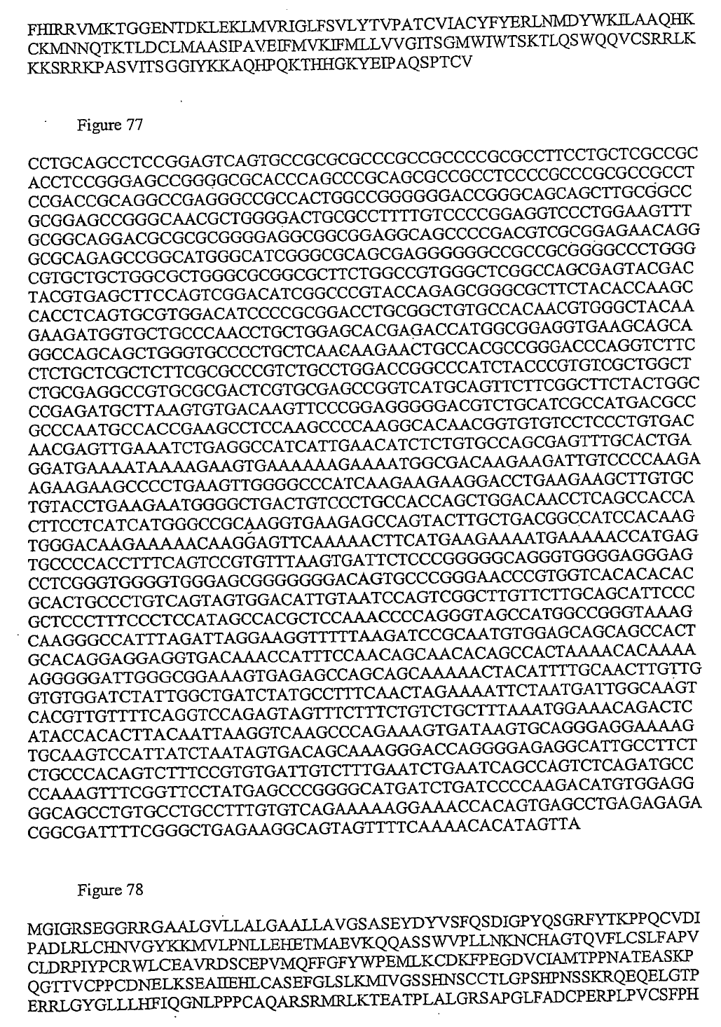

FIG. 77 represents the nucleic acid sequence of FRP;

FIG. 78 represents the amino acid sequence of FRP;

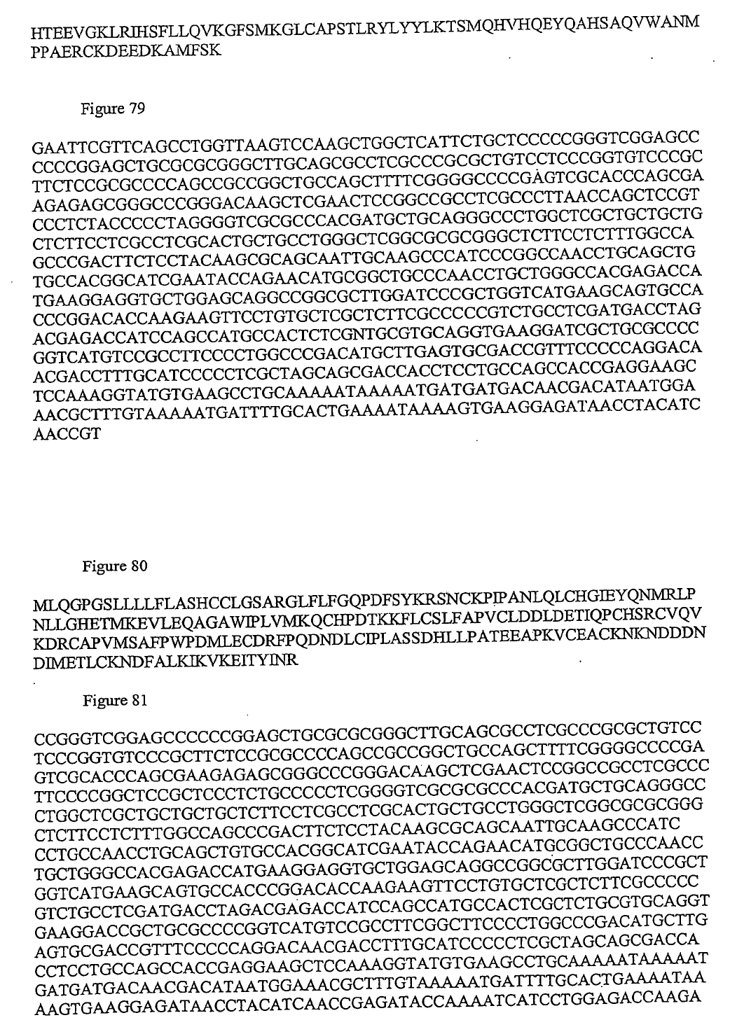

FIG. 79 represents the nucleic acid sequence of SARP 1;

FIG. 80 represents the amino acid sequence of SARP 1;

FIG. 81 represents the nucleic acid sequence of SARP 2;

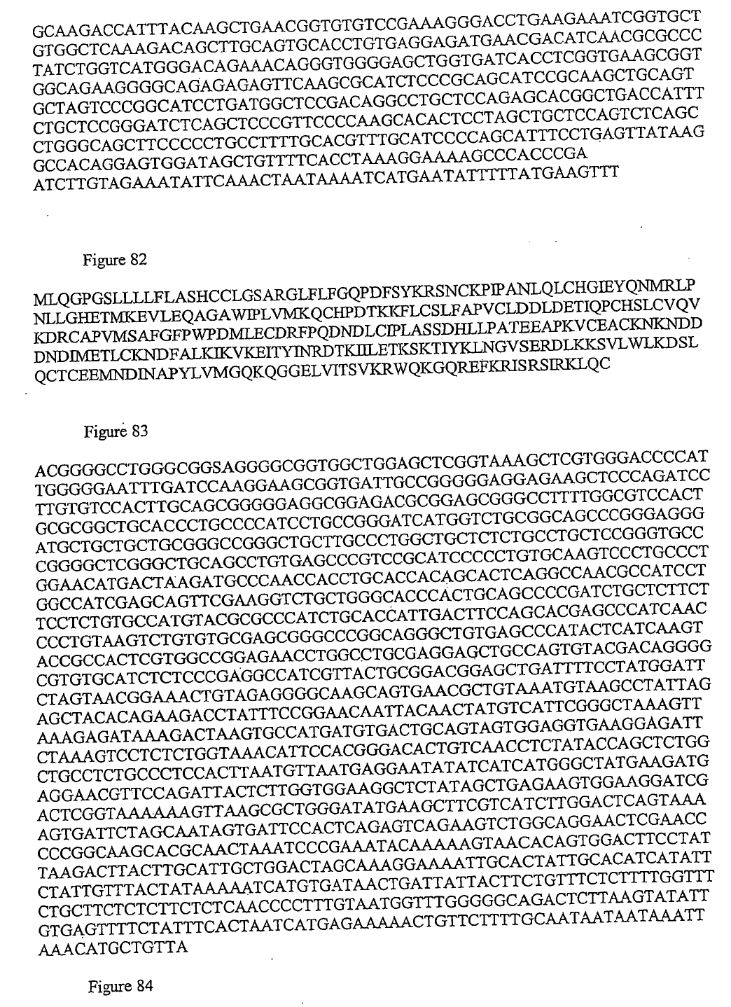

FIG. 82 represents the amino acid sequence of SARP 2;

FIG. 83 represents the nucleic acid sequence of FRZB;

FIG. 84 represents the amino acid sequence of FRZB;

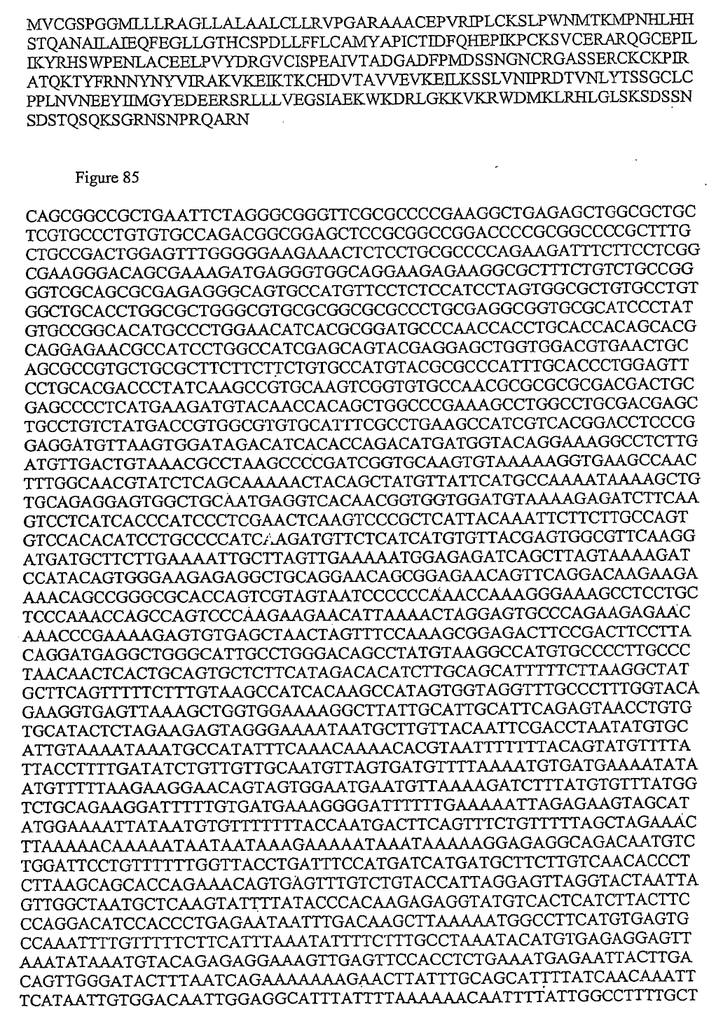

FIG. 85 represents the nucleic acid sequence of FRPHE;

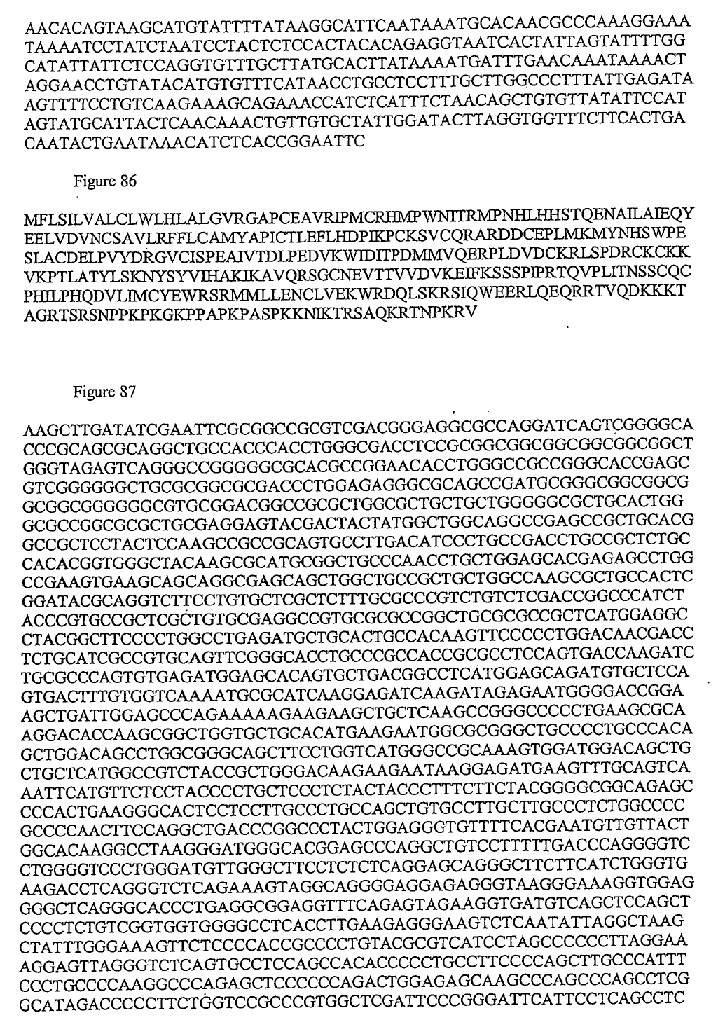

FIG. 86 represents the amino acid sequence of FRPHE;

FIG. 87 represents the nucleic acid sequence of SARP 3;

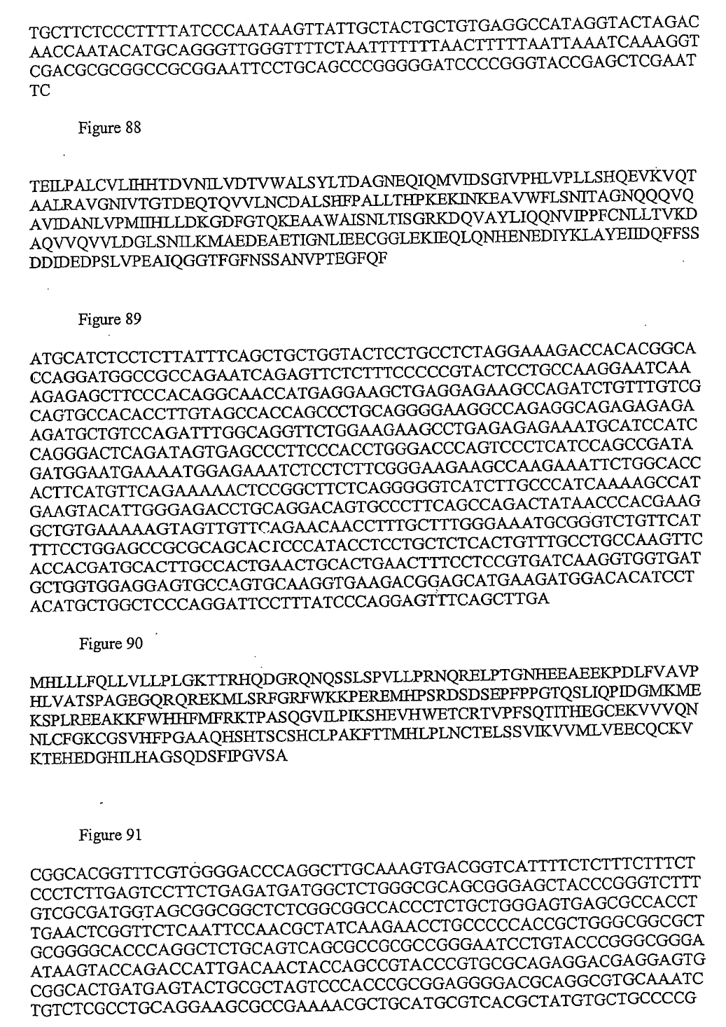

FIG. 88 represents the amino acid sequence of SARP 3;

FIG. 89 represents the nucleic acid sequence of CER 1;

FIG. 90 represents the amino acid sequence of CER 1;

FIG. 91 represents the nucleic acid sequence of DKK1;

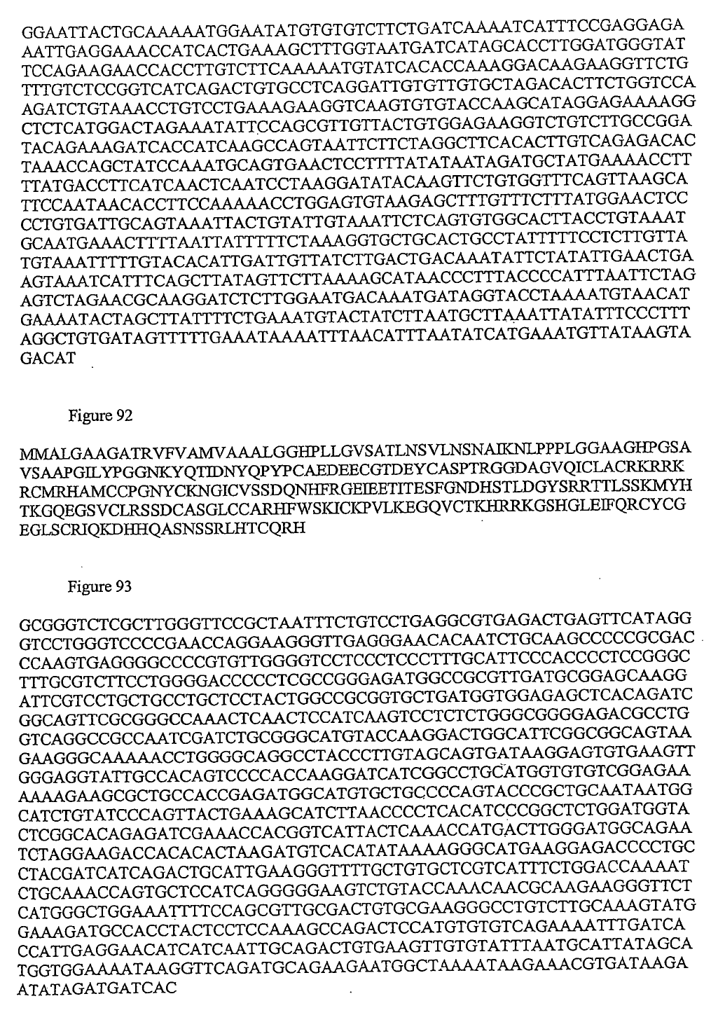

FIG. 92 represents the amino acid sequence of DKK1;

FIG. 93 represents the nucleic acid sequence of DKK2;

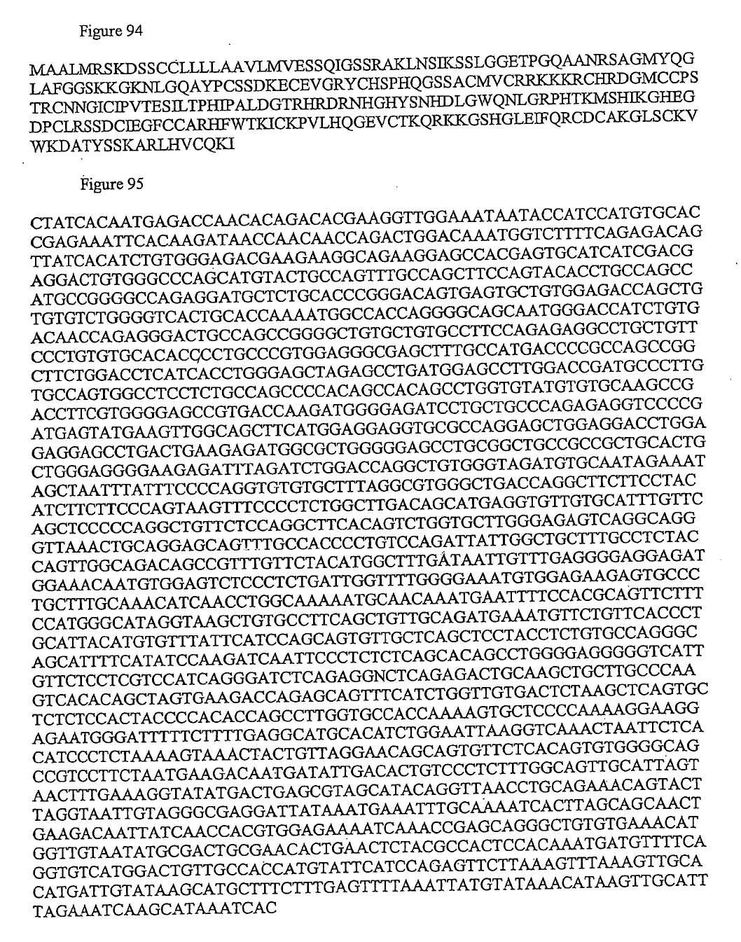

FIG. 94 represents the amino acid sequence of DKK2;

FIG. 95 represents the nucleic acid sequence of DKK3;

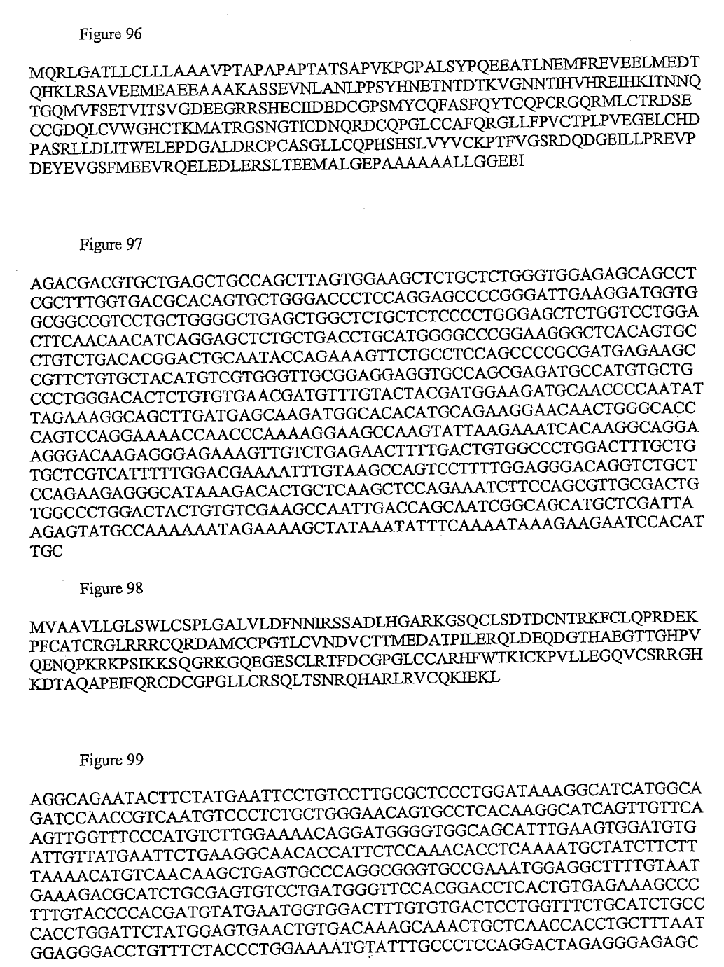

FIG. 96 represents the amino acid sequence of DKK3;

FIG. 97 represents the nucleic acid sequence of DKK4;

FIG. 98 represents the amino acid sequence of DKK4;

FIG. 99 represents the nucleic acid sequence of WIF-1;

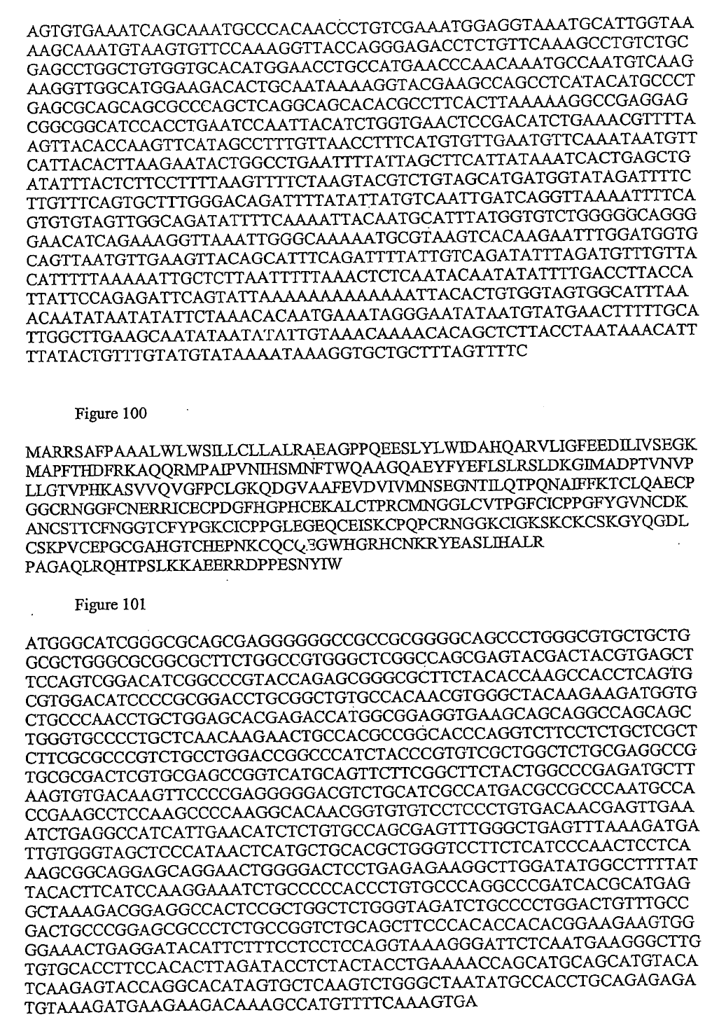

FIG. 100 represents the amino acid sequence of WIF-1;

FIG. 101 represents the nucleic acid sequence of SRFP 1;

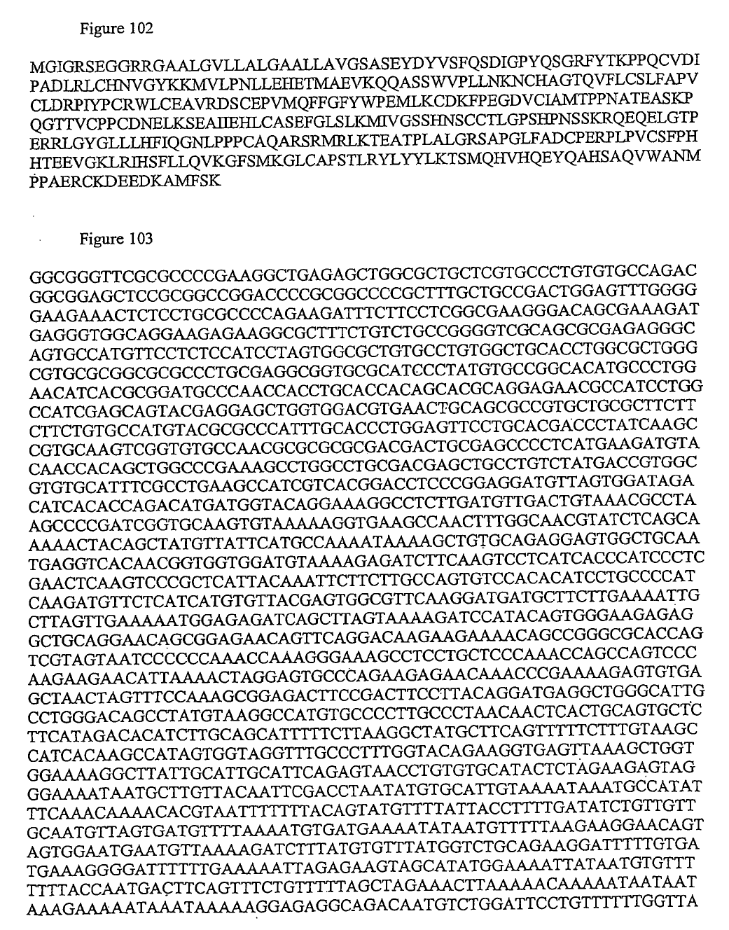

FIG. 102 represents the amino acid sequence of SRFP 1;

FIG. 103 represents the nucleic acid sequence of SRFP 4;

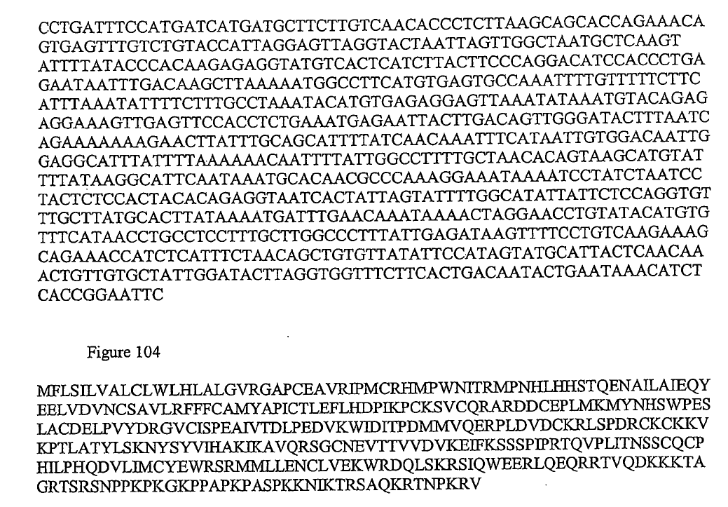

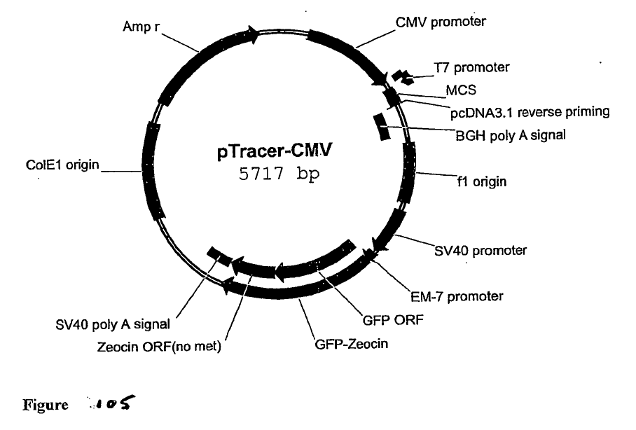

FIG. 104 represents the amino acid sequence of SRFP 4; and

FIG. 105 represents a diagram depicting the pCMV-tracer vector.

TABLE 1
Materials and Methods

| | | | | |
|---|---|---|---|---|
| | | | Xenograph | |
| Cell Line | Biopsy Site | Biopsy Histology | Histology | Reference |

Cell lines derived from germ cell tumours.

| | | | | |
|---|---|---|---|---|
| 2102Ep | Testis | EC, T, Y | EC | (Andrews et al., |
| | | | | 1980) |
| 833KE | Testis | EC, T, C, S | EC | (Andrews et al., |
| | | | | 1980) |
| TERA-1 | Lung | EC, T | | (Fogh and |
| | | | | Trempe, 1975) |
| NTERA2 cl. D1 | Lung | EC, T | EC, T | (Fogh and |
| | | | | Trempe, 1975) |
| | | | | (Andrews, 1984) |

Cell Lines derived from gestational choriocarcinomas.

| | | |
|---|---|---|
| BEWO | Corresponds to gestational choriocarcinoma | (Pattillo and Gay, |
| | | 1968) |

Abbreviations used:
EC, embryonal carcinoma,
T, teratoma,
S, seminoma,
C, choriocarcinoma,
Y, yolk-sac carcinoma

List of Antibodies Used

| Antibody | Reference | References |
|---|---|---|
| SSEA-3 | Andrews et. al., 1982 | 12 |
| SSEA-4 | Kannagi et. al., 1983 | 18 |
| Tra-1-60 | Andrews et. al., 1984 | 25 |
| Tra-1-81 | Andrews et. al., 1984 | 25 |
| Tra-2-54 | Andrews et. al., 1984 | 20 |
| Tra-2-49 | Andrews et. al., 1984 | 20 |
| A2B5 | Fenderson et. al., 1987 | |
| ME311 | Fenderson et. al., 1987 | |
| Vin-is-56 | Andrews et. al., 1990 | 44 |
| Vin-is-53 | Andrews et. al., 1990 | 44 |
| Vin-2PB-22 | Andrews et. al., 1990 | 44 |
| Thy-1 | Andrews et. at., 1983 | 10 |

Expression Vectors

The following mammlian expression vectors are used in the expression of ligands herein disclosed:

Purchased from Stratagene Inc. pExchange-1; pExchange-2; pExchange-3A, 3B, 3C; pExchange-4A, 4B, 4C; pExchange-5A, 5b,5C; pExchange-6A, 6B, 6C; pExchange module EC-hyg; pExchange module EC-Puro; pExchange module EC-Neo; pCMV-Script; pCMV-Tag1; pCMV-Tag2; pCMV-Tag3; pCMV-Tag4; pCMV-Tag5; pCMVLACI, pOPRSVI/MCS, pOPI3-CAT; pERV3; pEGSH.

Purchased from Invitrogen Inv.

T-REX System Vectors

pcDNA4/TO; pcDNA4/TO/myc-His; pcDNA6/TR; pT-Rex-DEST30; pT-Rex-DEST31; pcDNA4/TO-E; pcDNA5/FRT/TO; pcDNA5/FRT/TO-TOPO.

Geneswitch System vectors

pGene/V5-His A, B, C; pSwitch

Ecdysone-Inducible System

PVgRXR; pIND; pIND(SP1); pIND/V5-His; pIND/V5-His-TOPO; pIND/GFP; pInd(SP1)/GFP.

PShooter Vectors

pRF/Myc/Nuc; pCMV/Myc/nuc; pEF/myc/mito; pCMV/myc/mito; pEF/myc/ER; pCMV/myc/ER; pEF/myc/cyto; pCMV/myc/cyto.

Invitrogen Inc

pTet-off; pTet-on; ptTA-2//3/4; pTet-tTS; pTRE2hyg PTRE2pur; pTRE2; pLP-TRE2; PTRE-Myc; pTRE-HA; pTRE-6xHN pTRE-d2EGFP; pBI; pBI-EGFP; pBI-G; pBI-L; pTK-Hyg

“Living Colours” Vectors.

pDsRed2-N1; pDsRed2-C1; pECFP-N1; pEGFP-N1; pEGFP-N2; pEGFP-N3 pEYFP-N1; pECFP-C1; pEGFP-C1; pEGFP-C2; pEGFP-C3 pEYFP-C1; pd1EGFP-N1; pd1ECFP-N1; pd2EGFP-N1; pd2EYFP-N1 pd4EGFP-N1; pCMS-EGFP; pHygEGFP; pEGFPLuc; pNF-κB-dsEGFP pIRES2-EGFP; pIRES-EYFP

Maintenance of Cell Lines

All cells were grown in Dulbecco's modified Eagle's medium (DMEM), supplemented with 10% by volume foetal calf serum (Gibco BRL) and 2 mM L-glutamine. Tissue culture flasks were incubated in a humidified atmosphere of 10% CO₂ in air at 37° C.

Treatment of NTERA2 Cells

Retinoic Acid

Medium was aspirated from confluent flasks of EC cells and the cells rinsed in sterile PBS. 1 ml of 0.25% (w/v) trypsin in 2 mM EDTA was added per 75 cm² flask and the flask incubated at room temperature for up to 5 minutes. Vigorous shaking was subsequently used to dislodge the cells. Cells were suspended in 9 ml of supplemented DMEM per ml of trypsin used and counted in a haemocytometer. Cells were seeded at 10⁶ cells per 75 cm² flask, in medium containing 10⁻⁵M all-trans-retinoic acid (Eastman Kodak), diluted from a 10⁻²M stock solution in dimethyl sulfoxide (DMSO). Flasks were incubated as described above and the media replaced as and when required.

Hexamethylene Bisacetamide (HMBA)

Cells to be treated with HMBA were prepared as described for retinoic acid, but grown in medium supplemented with 10⁻³M HMBA instead of RA.

Harvesting of Cells

Cells were dislodged from the culture vessel with trypsin and suspended in 9 ml culture medium per ml of trypsin solution used, as described above. The cell suspension was then centrifuged at 400×g for 3 minutes and the medium aspirated from the resulting cell pellet. Cells were then rinsed in 5 ml PBS and centrifuged again at 400×g for 1 minute. The PBS rinse was aspirated and the cells stored at −80° C. or used immediately.

Total RNA Preparation

Where possible, all vessels and all solutions used in RNA preparation and storage were treated with a 0.01% (v/v) solution of diethylpyrocarbonate (DEPC) in distilled water, and subsequently autoclaved.

TR1 reagent (Sigma) was added to pelleted cells in a quantity corresponding to 1 ml per 75 cm² flask. The lysate was agitated until homogenous. 0.2 ml of chloroform was added per ml of TR1 reagent used and the vessel vortexed for 10 seconds. After 10 minutes at room temperature, the lysate was centrifuged at 12000×g for 15 minutes at 4° C. Following centrifugation, the aqueous (uppermost) phase was transferred to a fresh vessel and 0.5 ml of isopropanol added per ml of TR1 reagent used. The sample was incubated at room temperature for 10 minutes, then centrifuged at 12000×g for 10 minutes at 4° C. Following centrifugation, the supernatant was removed and the pellet washed in 70% ethanol. RNA was dissolved in DEPC-treated, double-distilled water.

Isolation of mRNA

100 mg oligo dT cellulose (Ambion) was suspended in 25 ml binding buffer. Up to 2 mg of total RNA was then added to the binding buffer and the suspension gently agitated at room temperature for 45 minutes. The suspension was then centrifuged at 3000×g for 10 minutes and the supernatant discarded. The resulting pellet was re-suspended in a further 25 ml of binding buffer and agitated at room temperature for 60 minutes. The suspension was again centrifuged at 3000×g and the supernatant discarded. The pellet of oligo dT cellulose was transferred to a spin column using a minimal quantity of binding buffer to re-suspend. The column was spun at maximum speed in a desktop microfuge for 30 seconds and the eluate discarded. This was repeated until the cellulose was dry. 200 μl of wash buffer was then added to the cellulose and mixed in with a pipette tip. The column was spun at maximum speed for 1 minute and the eluate discarded. 200 μl of DEPC-treated, double-distilled H₂O was then added to the cellulose and mixed in, as before. The column was then spun at maximum speed for 2 minutes and the eluted mRNA collected.

Precipitation of RNA

To the RNA solution was added 0.1× volume of 5M LiCl and 2.5× volume of 100% ethanol. After vortexing briefly, the sample was incubated at −20° C. for >60 minutes to precipitate. Precipitated RNA was centrifuged at maximum speed in a bench top microfuge for 30 minutes. The supernatant was discarded and the pellet rinsed in 70% ethanol, then dissolved in H₂O.

Quantitation of Nucleic Acid

A Beckman DU 650 spectrophotometer was used for the quantitation of both DNA and RNA. The machine was set to measure absorbence at wavelengths of 260 nm and 280 nm. After blanking the machine on an appropriate solution, diluted DNA or RNA samples in a volume of 100 μl were added to the cuvette and measured. The absorbence at 260 nm was used to calculate nucleic acid concentration in μg/μl, as shown below:

[Nucleic acid]=(A²⁶⁰×N×DF)÷1000

Where N is 33 for single-stranded DNA, 50 for double-stranded DNA and 40 for RNA and DF is the dilution factor for the sample added to the cuvette.

Northern Blot Analysis

Blot Preparation

1 g of agarose was dissolved in 85 ml H₂O by boiling. After cooling to around 70° C., 10 ml of 1033 MOPS buffer and 5 ml of formaldehyde were added, and the gel cast. 1-5 μg of each mRNA sample was mixed with an appropriate quantity of 10× RNA loading buffer to give a final volume of no more than 30 μl. The RNA was then denatured at 95° C. for 2 minutes and quenched on ice for 10 minutes. The gel was placed in an electrophoresis tank containing 1× MOPS buffer and the samples loaded into each well of the gel, along with appropriate molecular weight markers in the outermost wells. 80V were applied across the gel for 2-3 hours or as required. Following electrophoresis, the outermost lanes containing the molecular weight markers were removed using a scalpel and submerged in double-distilled H₂O containing ethidium bromide at 0.5 μg/ml. The remainder of the gel was submerged in >5 volumes of double-distilled H₂O, which was replaced every 5 minutes for a total of 25 minutes. An appropriately sized piece of GeneScreen Plus (DuPont) membrane, just larger than the area of gel to be blotted, was cut. The membrane was hydrated by briefly submerging in double-distilled H₂O, then transferred to 10×SSC, concurrent with the last 15 minutes of gel washing. The blotting apparatus was assembled as shown in FIG. 2.1, with the gel upside-down, using 10×SSC transfer buffer. After transfer of at least 6 hours, the absorbent material was removed from the membrane. After marking the position of the wells using a pencil, the membrane was removed from the gel and washed briefly in 2×SSC. Whilst still damp, the RNA was fixed to the membrane by UV crosslinking. The membrane was then baked at 80° C. for 3 hours.

The excised marker lanes were de-stained by soaking in a large volume of double-distilled H₂O for around 3 hours, after which they were visualised on a UV transilluminator and photographed.

Probe preparation

Random-primed DNA labelling was carried out using the Prime-a-Gene kit from Promega. Approximately 25 ng of template DNA (PCR or restriction digest product) was denatured at 95° C. for 2 minutes, then quenched on ice for 10 minutes. The reaction mix was then assembled on ice, in the order indicated below:

10 μl of 5×labelling buffer

H₂O to give a final volume of 50 μl

2 μl unlabelled DNTP mix (0.5 mM each)

0.25 ng of denatured/quenched template DNA

2 μl 10 mg/ml BSA

5 μl αP³²dATP 3000 Ci/mmol (NEN DuPont)

1 μl DNA polymerase 1 large (Klenow) fragment

The labelling reaction mix was incubated at room temperature for 2 hours. After this period, unincorporated nucleotides were removed using Pharmacia S-300 MicroSpin columns. Columns were placed in a microfuge tube and pre-spun at 735×g for 1 minute. The column was then transferred to a fresh tube and the entire labelling reaction added. The column was then spun at 735×g for a further 2 minutes and the purified, labelled DNA collected. Labelled DNA was denatured at 95° C. for 2 minutes, then quenched on ice for 15 minutes.,

Hybridisation and Washing Procedure

Northern blots were equilibrated in 150 ml of 2×SSC at 42° C. for 15 minutes in a hybridisation oven at 8 RPM. The SSC was exchanged for 25 ml of hybridisation buffer, pre-warmed to 42° C., and the filter incubated for a further 30 minutes at the same temperature. The entire volume of purified probe solution was then added to the hybridisation buffer and the blot incubated overnight at 42° C./8 RPM. The hybridisation solution was then discarded and the blot washed as follows:

2×SSC at room temperature for 20 minutes

2×SSC at room temperature for 20 minutes

2×SSC/1% SDS at 65° C. for 45 minutes

2×SSC/1% SDS at 65° C. for 45 minutes

0.1×SSC at room temperature for 20 minutes

0.1×SSC at room temperature for 20 minutes

Filters were exposed to a Bio Rad BI phosphor-imager screen overnight and, in most cases, subsequently exposed to X-ray film (Kodak X-omat AR).

Loading Controls for Northern Blots

All Northern blots used in this study were probed with β-actin as a loading control. Table 2.5 (overleaf) lists the figures to which each control probing (panel A to T, FIG. 2.2) corresponds. Northern blot data presented in this study have not, in all cases, been subject to repeat experiments using RNA isolated from different batches of cells. These data may not be regarded as conclusive, since reproducibility has not been proven.

Method for Analysis of the Requirement for Notch Ligands in the

Differentiation of Embryonic Stem, Embryonal Carcinoma and their Differentiated Derivatives.

CHO are transfected with constructs encoding either membrane bound or soluble forms of the Notch ligands. These cell lines are used to support the growth of either Embryonal carcinoma cells (EC) e.g NTERA2/c1.D1 or Human embryonic stem cells (hES).

The transfected CHO cells (CHO(DSL)) are used in the following way. To assess membrane bound forms of the Notch ligands the CHO(DSL) cells are used as feeder cells (i.e. the EC or hES will be grown on top of the CHO(DSL) cells). To assess the soluble forms of the Notch ligands either supernatant from the transfected CHO cells or concentrated ligand molecules derived from the supernatant are added to the culture medium of the EC and hES cells.

Notch Ligand Constructs.

The following cloned Notch ligands were obtained from Dr. Shigeru Chiba, Department of Hematology, Oncology and Cell Therapy, Transplantation Medicine. Graduate School of Medicine. University of Tokyo.

Delta1-FLAG

Jagged1-FLAG

Jagged2-FLAG

Soluble Delta1-Fc

Soluble Jagged1-Fc

Soluble Jagged2-Fc

These had been cloned into the vector pTRACER-CMV from Invitrogen, FIG. 30).

The clones used consisted either of the full length ligand linked to a histidine tag (FLAG, Kodak Inc.), or a ligand lacking the membrane spanning and intracellular portion of the protein thus rendering the ligand soluble. These had been linked to the Fc portion of human IgG.

Generation of Notch Ligand expressing Cell lines

The Chinese Hamster Ovary derived cell line AA8 was maintained in MEM Alpha medium with Glutamax-1 supplemented with ribonucleosides and deoxyribonucleosides (Lifetechnologies) and 10% Foetal Bovine Serum (FBS)(Lifetechnologies).

Plasmid was transfected into the AA8 cells using either Fugene (Roche) or Lipofectin (Lifetechnologies) or Superfect (Qiagen) according to manufacturers protocols.

Assessment of Transiently Transfected Cell Lines for Ligand Production.

Both soluble and membrane bound forms of the Notch ligand's production are assayed by western blotting and chemiluminesent detection.

Cells transfected with the ligand encoding constructs are harvested and the proteins extracted. Due to the tagging of the ligands proteins are able to be run out on an SDS-PAGE gel, blotted and probed with either mouse anti-FLAG antibody and detected using a anti-mouse HRP secondary or an HRP-secondary antibody. Both methods use electro-chemiluminecence (ECL) as the detection method.

Concentration of Soluble Notch Ligand From the Supernatant of Transfected CHO Cells.

Fc-labelled Notch ligand can be purified from transfected CHO cells supernatant using a HiTrap protein G HP column (Amersham Pharmacia Biotech). A sample can be analysed by western blotting as described above.

Embryonic Cell Culture.

Human Embryonal Carcinoma NTERA2/D1 cells are maintained in Dulbecco's modified Eagles medium (DMEM), supplemented with 2 mM 1-glutamine, 10% Foetal Bovine Serum (Lifetechnologies) and at 37° C. under 10% CO₂ in air. Cells were passaged by scraping from the surface of the tissue culture flask with 3 mm glass beads and reseeded at 5×10⁶ cells per 75 cm³ flask. For specific seeding densities cells were pasaaged using 0.25% Trypsin (Lifetechnologies) in Dulbecco's Phosphate Buffered Saline (PBS) supplemented with 1 mM EDTA.

Human Embryonic Stem Cells are maintained on irradiated mouse embryonic fibroblasts in serum free conditions, with 80% F12:DMEM (Lifetechnologies), 20% Knockout SR (Lifetechnologies), 1% Non-essential amino acid solution (Lifetechnologies), 1 mM L-glutamine, 0.1 mM β-mercaptoetanol (Sigma) 4 ng/ml bFGF (Sigma). The cells are passaged using collagenase IV and scraping.

Flow Cytofluorimetry

Cells were removed from their adherent culture surface and incubated with suitable primary antibody for 1 hour at 4C. Cells are washed in PBS with 5% FCS and incubated for a further hour with a suitable FITC-conjugated labelled secondary antibody, and analysed on a EPICS Elite ESP Flow Cytometer (Coulter Electronics). Colonies were assessed for the presence of embryonal stem cell markers such as SSEA-3, -4, Tra-1-60 and for appearance of markers of differentiated marker antigens such as A2B5, ME311 and N901.

Design of Oligonucleotide Primers

Primers for use in PCR were designed on a Macintosh Power PC, using the “Primer Select” program of the DNASTAR software package (DNASTAR Inc.). All primers used in this study are shown in Table 2

TABLE 2

List of oligonucleotide primers

| | GenBank | Primer | Prinmer | |
|---|---|---|---|---|
| Gene | accession | direction | location | Primer sequence 5′ to 3′ |
| Wnt-13 | Z71621 | Forward | 1159-1178 | Tgagtggttcctgtactctg |
| | | Reverse | 1503-1484 | Actcacactgggtaacacgg |
| SFRP4 | XM_004706 | Forward | 858-880 | Agaggagtggctgcaatgaggtc |
| | | Reverse | 1159-1142 | Gcgcccggctgttttctt |
| Waf1 | U03106 | Forward | 487-506 | Cagggtcgaaaacggcggca |
| | | Reverse | 947-928 | Aggagccacacccctccaga |
| β-actin | NM_001101 | Forward | 326-357 | Atctggcaccacaccttctacaatgagctgc |
| | | | | g |
| | | Reverse | 1163-1132 | Cgtcatactcctgcttgctgatccacatctgc |
| neuroD1 | NM_002500 | Forward | 240-263 | Aagccatgaacgcagaggaggact |
| | | Reverse | 818-799 | Agctgtccatggtaccgtaa |

All PCR data presented in this study were duplicated in independent experiments to eliminate the possibility of methodological error. However, duplicate experiments were performed on identical samples and do not, therefore, control for variability between separate batches of cells. Polymerase chain reactions from which quantitative interpretations were to be made were controlled by parallel amplification of the cyclin-dependent kinase inhibitor, Waf1. This transcript has been demonstrated by other workers in the laboratory to be constitutively expressed by NTERA2 EC cells and differentiated derivatives (unpublished data). Furthermore, Waf1 has been shown to exhibit an approximately 20-fold lower abundance in the NTERA2 system than the more widely used control, β-actin, and is therefore well suited to the analysis of rare transcripts.

PCR Reaction Conditions

PCR mixes were assembled on ice, with the following components per reaction:

5 μl of 25 mM MgCl₂

5 μl of 10× reaction buffer

5 μl of 1 mM dNTPs

3 μL of forward primer at 5 pmol/μl

3 μl of reverse primer at 5 pmol/μl

0.3 μl of Taq polymerase at 1 unit/μl (Promega)

template and H₂O to give 50 μl final volume

A premix was made containing all reaction components bar the template. Premix was then added to the reaction vessels containing the template, on ice. The reaction vessels were then transferred to the thermal cycler. The PCR programs used are shown in Table 3, with cycling from T1 [symbol] T2 [symbol] T3 [symbol] T1.

TABLE 3
PCR thermal cycling programs

| | Program 1 | Program 2 | Program 3 | Program 4 |
|---|---|---|---|---|
| T1 | 96° C./ | 94° C./ | 94° C./ | 95° C./ |
| (temp/duration) | 30 seconds | 60 seconds | 90 seconds | 90 seconds |
| T2 | 50° C./ | 55° C./ | 60° C./ | 63° C./ |
| (temp/duration) | 15 seconds | 90 seconds | 90 seconds | 60 seconds |
| T3 | 60° C./ | 72° C./ | 72° C./ | 72° C./ |
| (temp/duration) | 240 seconds | 60 seconds | 120 seconds | 60 seconds |
| Cycles | 25 | 35 | 35 | 35 |

List of DNA and protein accession
numbers of genes used in results

| | | | Protein |
|---|---|---|---|
| Gene | | cDNA | Accession |
| Name | Description | Accession Number | Number |
| WNT2B | wingless-type MMTV | AB045116 | Q93097 |
| | integration site family, | | |
| | member 2B | | |
| | member 2B | | |
| SFRP1 | secreted frizzled- | AF056087 | AAC12877 |
| | related protein 1 | | |
| SFRP4 | secreted frizzled- | AF026692 | AAC04617 |
| | related protein 4 | | |
| FRZB | frizzled-related protein | NM_001463 | NP_001454 |
| SFRP2 | secreted frizzled- | | |
| | related protein 2 | | |
| FZD1 | frizzled (Drosophila) | AB017363 | BAA34666 |
| | homolog 1 | | |
| FZD2 | frizzled (Drosophila) | NM_001466 | NP_001457 |
| | homolog 2 | | |
| FZD9 | frizzled (Drosophila) | HSU82169 | AAC51174 |
| | homolog 9 | | |
| FZD3 | frizzled (Drosophila) | Kirikoshi et. al., 2000 | Kirikoshi |
| | homolog 3 | | et. al., 2000 |
| FZD5 | frizzled (Drosophila) | | |
| | homolog 5 | | |
| FZD4 | frizzled (Drosophila) | NM_012193 | NP_036325 |
| | homolog 4 | | |
| FZD6 | frizzled (Drosophila) | AB012911 | BAA25686 |
| | homolog 6 | | |
| FZD7 | frizzled (Drosophila) | AB017365 | BAA34668 |
| | homolog 7 | | |
| DVL2 | dishevelled 2 | NM_004422 | NP_004413 |
| | (homologous to | | |
| | Drosophila dsh) | | |
| DVL3 | dishevelled 3 | NM_004423 | NP_004414 |
| | (homologous to | | |
| | Drosophila dsh) | | |
| GSK3B | glycogen synthase | NM_002093 | NP_002084 |
| | kinase 3 bets | | |
| AXIN1 | axin | AF009674 | AAC51624 |
| APC | adenomatosis | NM_000038 | NP_000029 |
| | polyposis coli | | |
| TCF1 | transcription factor 1, | M57732 | AAA88077 |
| | hepatic; LF-B1, | | |
| | hepatic nuclear factor | | |
| | (HNF1), albumin | | |
| | proximal factor | | |

### EXAMPLES

Expression of a single Wnt gene, Wnt-13(2B) was detected. This transcript was absent in NTERA2 EC cells, but showed marked up-regulation following RA treatment, FIG. 24. Members of the FRP family, encoding putative Wnt antagonists, also showed altered expression during differentiation, FIG. 24. Both Frp-1 and SARP-1 were down-regulated following RA treatment, whilst FrpHE was absent in EC cells, but expressed at high levels in RA treated cultures.

Several members of the frizzled family were also detected, providing a candidate receptor system for Wnt-13, FIG. 24. Two of these, hFz-4 and hFz-6, showed developmental regulation. Transcripts corresponding to intracellular components of the Wnt pathway, including Dishevelled, GSK-3b, Axin, APC and TCF were present at equivalent levels in EC and differentiating cultures. CBP was also ubiquitously expressed.

### REFERENCES

1. Andrews P. W. and Roberts D. B. (1974) The preparation and characterization of chromatin from third instar larvae of Drosophila melanogaster. Nucleic Acids Res. 1: 979-997.

2. Roberts D. B. and Andrews P. W. (1975) Drosophila chromatin: An immunological study. Nucleic Acids Res. 2:1291-1303.

3. Andrews P. W. and Boyse E. A. (1978) Mapping of an H-2-linked gene that influences mating perference in mice. Immunogenetics 6:265-268.

4. Yamazaki K., Yamaguchi M., Andrews P. W., Peake B. and Boyse E. A. (1978) Mating preferences in F₂ segregants of crosses between MHC-congenic mouse strains. Immunogenetics 6: 253-259.

5. Andrews P. W. and Wachtel S. S. (1979) Rejection of C57BL skin grafts by (C57BI×Mus musculus castaneus) F₁ hybrids. Transplantation 27: 4344.

6. Andrews P. W. and Goodfellow P. N. (1980) Antigen expression by somatic cell hybrids of a murine embryonal carcinoma cell with thymocytes and L cells. Somat. Cell Genet. 6: 271-284.

7. Bronson D. L., Andrews P. W., Solter D., Cervenka J., Lange P. H. and Fraley E. E. (1980) A cell line derived from a metastasis of a human testicular germ-cell tumor. Cancer Res. 40: 2500-2506.

8. *Andrews P. W., Bronson D. L., Benham F., Strickland S. and Knowles B. B. (1980) A comparative study of eight cell lines derived from human testicular teratocarcinoma. Int. J. Cancer 26: 269-280.

9. Andrews P. W., Knowles B. B. and Goodfellow P. N. (1981) A human cell surface antigen defined by a monoclonal antibody and controlled by a gene on chromosome 12. Somat. Cell Genet. 7: 435-443.

10. Andrews P. W., Bronson D. L., Wiles M. V. and Goodfellow P. N. (1981) The expression of major histocompatibility antigens by human teratocarcinoma derived cells lines. Tissue Antigens 17: 493-500.

11. Benham F. J., Andrews P. W., Bronson D. L., Knowles B. B. and Harris H. (1981) Alkaline phosphatase isozymes as possible markers of differentiation in human teratocarcinoma cell lines. Dev. Biol. 88: 279-287.

12 Andrews P. W., Goodfellow P. N., Shevinsky L., Bronson D. L. and Knowles B. B. (1982) Cell surface antigens of a clonal human embryonal carcinoma cell line: Morphological and antigenic differentiation in culture. Int. J. Cancer 29: 523-531.

13. Andrews P. W. (1982) Human embryonal carcinoma cells in culture do not synthesize fibronectin until they differentiate. Int. J. Cancer 30: 567-571.

14. Damjanov I. and Andrews P. W. (1983) Ultrastructural differentiation of a clonal human embryonal carcinoma cell line iii vitro. Cancer Res. 43: 2190-2198.

15. Matthaei K., Andrews P. W. and Bronson D. L. (1983) Retinoic acid fails to induce differentiation in human teratocarcinoma cell lines that express high levels of cellular receptor protein. Exp. Cell Res. 143: 471-474.

16. Cossu G., Andrews P. W. and Warren L. (1983) Covalent binding of lactosaminoglycans and heparan sulphate to fibronectin synthesized by a human teratocarcinoma cell line. Biochem. Biophys. Res. Comm. 111: 952-957.

17. Tunnacliffe A., Goodfellow P. N., Banting G., Solomon E., Knowles B. B. and Andrews P. W. (1983) Human chromosome 11 carries at least 4 genes controlling expression of cell surface antigens. Somat. Cell Genet. 9: 629-642.

18. Kannagi R., Cochran N. A., Ishigami F., Hakomori S.-i., Andrews P. W., Knowles B. B. and Solter D. (1983a) Stage-specific embryonic antigens (SSEA-3 and -4) are epitopes of a unique globo-series ganglioside isolated from human teratocarcinoma cells. The EMBO J. 2: 2355-2361.

19. *Andrews P. W., Damjanov I., Simon D., Banting G., Carlin C., Dracopoli N. C. and Fogh J. (1984b) Pluripotent embryonal carcinoma clones derived from the human teratocarcinoma cell line Tera-2: Differentiation in vivo and in vitro. Lab. Invest. 50: 147-162.

20. Andrews P. W., Meyer L. J., Bednarz K. L. and Harris H. (1984c) Two monoclonal antibodies recognizing determinants on human embryonal carcinoma cells react specifically with the liver isozyme of human alkaline phosphatase. Hybridoma 3: 33-39.

21. *Gönczöl E., Andrews P. W. and Plotkin S. A. (1984) Cytomegalovirus replicates in differentiated but not undifferentiated human embryonal carcinoma cells. Science 224: 159-161.

22. *Andrews P. W. (1984) Retinoic acid induces neuronal differentiation of a cloned human embryonal carcinoma cell line in vitro. Dev. Biol. 103: 285-293.

23. Oosterhuis J. W., Andrews P. W., Knowles B. B. and Damjanov I. (1984) Effects of cisplatinum on embryonal carcinoma cell lines in vitro. Int. J. Cancer 34: 133-139.

24. Blaineau C., Connan F., Amaud D., Andrews P. W., Williams L., McIlhinney R. A. J. and Avner P. (1984) Definition of three species-specific monoclonal antibodies recognizing antigenic structures present on human EC cells which undergo modulation during in vitro differentition. Int. J. Cancer 34: 487-494.

25. Andrews P. W., Banting G. S., Damjanov I., Arnaud D. and Avner P. (1984a) Three monoclonal antibodies defining distinct differentiation antigens associated with different high molecular weight polypeptides on the surface of human embryonal carcinoma cells. Hybridoma 3: 347-361.

26. Damjanov I., Clark C. K. and Andrews P. W. (1984) Cytoskeleton of human embryonal carcinoma cells. Cell Differentiation 15: 133-139.

27. Andrews P. W., Knowles B. B., Parkar M., Pym B., Stanley K. and Goodfellow P. N. (1985) A human cell-surface antigen defined by a monoclonal antibody and controlled by a gene on human chromosome 1. Ann. Human Genet. 49: 31-39.

28. Gönczöl E., Andrews P. W. and Plotkin S. A. (1985) The replication of human cytomegalovirus in human teratocarcinoma cell lines. J. Gen. Virol. 66: 509-515.

29. Damjanov I., Damjanov A. and Andrews P. W. (1985) Trophectodermal carcinoma: Mouse teratocarcinoma-derived tumor stem cells differentiating into trophoblastic and yolk sac elements. J. Embryol. Exp. Morph. 86: 125-141.

30. Carlin C. R. and Andrews P. W. (1985) Human embryonal carcinoma cells express low levels of functional receptor for epidermal growth factor. Exp. Cell. Res. 159: 17-26.

31. Andrews P. W., Damjanov I., Simon D. and Dignazio M. (1985) A pluripotent human stem cell clone isolated from the TERA-2 teratocarcinoma line lacks antigens SSEA-3 and SSEA-4 in vitro but expresses these antigens when grown as a xenograft tumor. Differentiation 29: 127-135.

32. Lee V. M-Y. and Andrews P. W. (1986) Differentiation of NTERA-2 clonal human embryonal carcinoma cells into neurons involves the induction of all three neurofilament proteins. J. Neurosci. 6: 514-521.

33. Andrews P. W., Gönczöl E., Plotkin S. A., Dignazio M. and Oosterhuis J. W. (1986) Differentiation of TERA-2 human embryonal carcinoma cells into neurons and HCMV permissive cells: Induction by agents other than retinoic acid. Differentiation 31: 119-126.

34. Tippett P., Andrews P. W., Knowles B. B. Solter D. and Goodfellow P. N. (1986) Red cell antigens P (globoside) and Luke: Identification by monoclonal antibodies defining the murine stage-specific embryonic antigens-3 and -4 (SSEA-3 and 4). Vox Sang. 51: 53-56.

35. Swallow D. M., Povey S., Parkar M., Andrews P. W., Harris H., Pym B. and Goodfellow P. N. (1986) Mapping of the gene coding for the human liver/bone/kidney isozyme of alkaline phosphatase to chromosome 1. Ann. Human Genet. 50: 229-235.

36. Andrews P. W., Trinchieri G., Perussia B. and Baglioni C. (1987) Induction of class 1 major histocompatibility complex antigens in human teratocarcinoma cells by interferon without induction of differentiation, growth inhibition or resistance to viral infection. Cancer Res. 47: 740-746.

37. *Fenderson B. A., Andrews P. W., Nudelman E., Clausen H. and Hakomori S.-i. (1987) Glycolipid core structure switching from globo- to lacto- and ganglio-series during retinoic acid-induced differentiation of TERA-2-derived human embryonal carcinoma cells. Dev. Biol. 122: 21-34.

38. Zhang X.-Y., Loflin P. T., Gehrke C. W., Andrews P. W. and Ehrlich M. (1987) Hypermethylation of human DNA sequences in embryonal carcinoma cells and somatic tissues but not in sperm. Nucleic Acids Res. 15: 9429-9449.

39. Mavilio F., Simeone A., Boncinelli E. and Andrews P. W. (1988) Activation of four homeobox gene clusters in human embryonal carcinoma cells induced to differentiate by retinoic acid. Differentiation 37: 73-79.

40. Williams B. P., Daniels G. L., Pym B., Sheer D., Povey S., Okubo Y., Andrews P. W. and Goodfellow P. N. (1988) Biochemical and genetic analysis of the OKa blood group antigen. Immunogenetics 27: 322-329.

41. Rendt J., Erulkar S. and Andrews P. W. (1989) Presumptive neurons derived by differentiation of a human embryonal carcinoma cell line exhibit tetrodotoxin-sensitive sodium currents and the capacity for regenerative responses. Exp. Cell Res. 180: 580-584.

42. Chen C., Fenderson B. A., Andrews P. W. and Hakomori S.-i. (1989) Glycolipid-glycosyltransferases in human embryonal carcinoma cells during retinoic acid-induced differentiation. Biochemistry 28: 2229-2238.

43. Andrews P. W., Göncözl E., Fenderson B. A., Holmes E. H., O'Malley G., Hakomori S.-i and Plotkin S. A. (1989). Human cytomegalovirus induces stage-specific embryonic antigen-1 in differentiating human teratocarcinoma cells and fibroblasts. J. Exp. Med. 169:1347-1359.

44. Andrews P. A., Nudelman E., Hakomori S.-i. and Fenderson B. A. (1990). Different patterns of glycolipid antigens are expressed following differentiation of TERA-2 human embryonal carcinoma cells induced by retinoic acid, hexamtehylene bisacetamide MA) or bromodeoxyuridine (BUdR). Differentiation 43: 131-138.

45. *Simeone A., Acampora D., Arcioni L., Andrews P. W., Boncinelli E. and Mavilio F. (1990). Sequential activation of human HOX2 homeobox genes by retinoic acid in human embryonal carcinoma cells. Nature 346: 763-766.

46. Hirka G., Prakesh K., Kawashima H., Plotkin S. A., Andrews P. W. and Gonczol E. (1991). Differentiation of human embryonal carcinoma cells induces human immunodeficiency virus permissiveness which is stimulated by human cytomegalovirus coinfection. J. Virol. 65: 2732-2735.

47. Marrink J., Andrews P. W., van Brummen P. J., de Jong H. J., Sleijfer D., Schraffordt-Koops H. and Oosterhuis J. W. (1991). TRA-1-60: A new serum marker in patients with germ cell tumors. IEt. J. Cancer 49: 368-372.

48. Zeichner S. L., Hirka G., Andrews P. W. and Alwine J. C. (1992). Differentiation-dependent HIV LTR regulatory elements active in human teratocarcinoma cells. J. Virol. 66: 2268-2273.

49. Fenderson B. A., Radin N. and Andrews P. W. (1993) Differentiation antigens of human germ cell tumors: distribution of carbohydrate epitopes on glycolipids and glycoproteins analysed using PDMP, an inhibitor of glycolipid synthesis. European Urology. 23: 30-37.

50. Giwercman, A., Andrews, P. W., Jorgensen, N., Muller, J., Graem, N., Skalkebaek, N. E. (−1993) Immunochemical expression of embryonal marker TRA-1-60 in carcinoma in situ germ cells and in testicular germ cell tumours. Cancer, 72: 1308-1314.

51. Rideg K., Hirka G., Pralcash K., Bushar L., Nothias, J-Y, Weinrann R., Andrews P. W. and Gonczbl E. (1994) DNA binding proteins that interact with the 19-base pair (CRE-like) element from the HCMV immediate early promoter in differentiating human embryonal carcinoma cells. Differentiation, 56: 119-129.

52. Wenk, J., Andrews, P. W., Casper, J., Hata, J-I., Pera, M. F., von Keitz, A., Damjanov, I., Fenderson, B. A. 1994. Glycolipids of germ cell tumours: extended globo-series glycolipids are a hallmark of human embryonal carcinoma cells. Int. J. Cancer. 58: 108-115.

53. Ackerman S. L., Knowles B. B., Andrews P. W. (1994). Gene regulation during neuronal and non-neuronal differentiation of NTERA2 human teratocarcinoma-derived stem cells. Mol. Brain Res. 25: 157-162.

54. *Andrews P. W., Damjanov I., Berends J., Kumpf S., Zappavingna V. Mavilio F. and Sampath K. (1994). Inhibition of proliferation and induction of differentiation of pluripotent human embryonal carcinoma cells by osteogenic protein-1 (or bone morphogenetic protein-7). Laboratory Investigation 71: 243-251.

55. Damjanov, I., Zhu, Z. M., Andrews, P. W., Fenderson, B. A. (1994). Embryonal carcinoma cells differentiate into parietal endoderm via an intermediate stage corresponding to primitive endoderm. In Vivo 8: 967-974.

56. Squires, P. E., Wakeman, J. A., Chapman, H., Kumpf, S., Fiddock, M. D., Andrews, P. W. and Dunne, M. J. (1996). Regulation of intracellular Ca²⁺ in response to muscarinic and glutamate receptor agonists during the differentiation of NTERA2 human embryonal carcinoma cells into neurons. European Journal of Neuroscience 8: 783-793.

57. Andrews, P. W., Casper, J., Damjanov, I., Duggan-Keen, M., Giwercman, A., Hata, J. I., von Keitz, A., Looijenga, L. H. J., Millán, J. L., Oosterhuis, J. W., Pera, M., Sawada, M., Schmoll, H. J., Skakkaebaek, N. E., van Putten, W. and Stern, P. (1996). Comparative analysis of cell surface antigens expressed by cell lines derived from human germ cell tumours. Int. J. Cancer 66: 806-816.

58. Gels, M. E., Marrink J, Visser, P., Sleijfer, D. T., Droste J. H. J., Hoekstra, H. J., Andrews, P. W., Koops, H. S. (1997). Importance of a new tumour marker TRA-1-60 in the follow-up of patients with clinical state I nonseminomatous testicular germ cell tumours. Annals of Surgical Oncology 4; 321-327.

59. Wakeman, J. A., Heath, P. R., Pearson, R. C. A., Andrews, P. W. (1997) MAL mRNA is induced during the differentiation of human embryonal carcinoma cells into neurons, and is also localised within specific regions of the human brain. Differentiation 62:97-105.

60. *Wakeman, J. A., Walsh, J., Andrews, P. W., (1998). Human Wnt-13 is developmentally regulated during the differentiation of NTERA-2 pluripotent human embryonal carcinoma cells. Oncogene 17:179-186

61. Giesberts, A. N., Duran, C., Morton, I. E., Piggot, C., White, S. J., Andrews, P. W. (1999). The expression and function of cadherin-mediated cell-to-cell adhesion in human embryonal carcinoma cells. Mechanisms of Development 83 115-125.

62. *Badcock, G., Pigott, C., Goepel, J., Andrews, P. W. (1999). The Human Embryonal Carcinoma Marker Antigen TRA-1-60 Is A Sialylated Keratan Sulphate Proteoglycan. Cancer Research 59 4715-4719.

63. Gokhale, P. J., Giesberts, AN., Andrews, P. W. (2000). Brachyury is Expressed by Human Teratocarcinoma Cells in the Absence of Mesodermal Differentiation. Cell Growth and Differentiation 11 157-162.

64 *Przyborski, S. A., Morton, I. E., Wood, A., Andrews, P. W. (2000) Developmental Regulation of Neurogenesis in the Pluripotent Human Embryonal Carcinoma Cell Line NTERA-2. Eur. J. Neurosci. 12: 3521-3528.

65. Andrews P. W. and Knowles B. B. (1982) Human teratocarcinoma: Tools for human embryology In: Teratocarcinoma and Embryonic Cell Interactions (T. Murumatsu, G. Gachelin, A. A. Moscona, and Y. Ikawa, eds). Japan Scientific Societies Press, Tokyo, pp 19-30.

66 Andrews P. W., Knowles B. B., Cossu G. and Solter D. (1982) Teratocarcinoma and mouse embryo cell surface antigens: Characterization of the molecule(s) carrying the SSEA-1 antigenic determinant. In: Teratocarcinoma and embryonic Cell Interactions (T. Murumatsu, G. Gachelin, A. A. Moscona and Y. Ikawa eds). Japan Scientific Societies Press, Tokyo, pp 103-119.

67 Goodfellow P. N. and Andrews P. W. (1982) Sexual differentiation and H-Y antigen(s). Nature, News and Views 295: 11-13.

68. Andrews P. W. and Goodfellow P. N. (1982) Analysing the mouse T/t complex. Nature, News and Views 299: 296-297.

69. Goodfellow P. N. and Andrews P. W. (1982) The biology of teratocarcinomas. (Meeting Report). Nature, News and Views 300: 107-108.

70. Andrews P. W. (1983) The characteristics of cell lines derived from human germ cell tumors. In: The Human Teratomas: Experimental and Clinical Biology (I. Damjanov, B. B. Knowles and D. Solter eds). Humana Press, Clifton, N. J., pp 285-311.

71. Benham F. I., Wiles, M. V., Banting G., Andrews P. W. and Goodfellow P. N. (1983) Human-mouse teratocarcinoma hybrids: A tool for analysis of gene activity in early human development. In: Human Teratomas: Experimental and Clinical Biology (I. Damjanov, B. B. Knowles and D. Solter, eds.). Humana Press, Clifton, N. J., pp 313-314.

72. Andrews P. W., Goodfellow P. N. and Damjanov I. (1983) Human teratocarcinoma cells in culture. Cancer Surveys 2: 41-73.

73. Goodfellow P. N. and Andrews P. W. (1983) Is there a human T/t locus? Nature, News and Views 302: 657-658.

74. Andrews P. W., Goodfellow P. N. and Bronson D. L. (1983) Cell surface characteristics and other markers of differentiation of human teratocarcinomas in culture. In: Teratocarcinoma Stem Cells. Cold Spring Harbor Conferences on Cell Proliferation, Vol. 10 (L. M. Silver, G. R. Martin and S. Strickland, eds.) pp 579-590.

75. Goodfellow P. N., Benham F., Andrews P. W., Trowsdale J., Lee J. and Quintero M. (1983) Developmental genetics of MHC expression using human-mouse hybrid cell lines. In: Teratocarcinoma Stem Cells. Cold Spring Harbor Conferences on Cell Proliferation, Vol. 10 (L. M. Silver, G. R. Martin and S. Strickland, eds.), pp 439449.

76. Bronson D. L., Andrews P. W., Vessella R. L. and Fraley E. E. (1983) In vitro differentiation of human embryonal carcinoma cells. In: Teratocarcinoma Stem Cells. Cold Spring Harbor Conferences on Cell Proliferation, Vol. 10 (L. M. Silver, G. R. Martin and S. Strickland, eds.), pp 597-605.

77. Andrews P. W. (1984) The male specific antigen (H—Y) and sexual differentiation. In: Genetic Analysis of the Cell Surface (P. N. Goodfellow, ed.). Chapman and Hall, London, pp 159-190.

78. Andrews P. W. and Damjanov I. (1985) Immunochemistry of human teratocarcinoma stem cells. In: Monoclonal Antibodies in Cancer (S. Sell and R. A. Reisfeld, eds.). The Humana Press Inc., Clifton, N. J., pp 339-364.

79. Andrews P. W. (1985) Properties of cloned human embryonal carcinoma cells and their differentiation in vitro. In: Germ Cell Tumors II: Proceedings of the 2nd Germ Cell Tumor Conference, Leeds (W. G. Jones, A. Milford-Ward and C. K. Anderson, eds.). Pergamon Press, Oxford, pp 71-75.

80. Damjanov I., Clark R. K. and Andrews P. W. (1985) Expression of keratin polypeptides in human embryonal carcinoma cells. Ann. NY Acad. Sci. 455: 732-733.

81. Oosterhuis J. W., Andrews P. W. and de Jong, B. (1986) Mechanisms of therapy related differentiation in testicular germ cell tumors. In: Biochemical Mechanisms of the Platinum Anti-tumor Drugs (D. C. H. McBrien and T. F. Slater, eds.). Proceedings of an Association for International Cancer Research Symposium. IRL Press, Oxford, pp 65-90.

82. Andrews, P. W., Oosterhuis J. W. and Damjanov I. (1987) Cell lines from human germ cell lines. In: Teratocarcinomas and embryonic stem cells: A practical approach (E. J. Robertson, ed.). IRL Press, Oxford, pp 207-248.

83. Andrews P. W., Fenderson B. A. and Hakomori S.-i. (1987) Human embryonal carcinoma cells and their differntiation in culture. Int. J. Androl. 10: 95-104.

84. Andrews P. W. (1987) Human teratocarcinoma stem cells: Glycolipid antigen expression and modulation during differentiation. J. Cell Biochem. 35: 321-332.

85. Andrews P. W. (1988) The properties of human teratocarcinoma in vitro. In In Vitro Models for Cancer Research (M. Webber and L. Sekely, eds.). CRC Press, Boca Raton, Fla., pp 191-213.

86. Andrews P. W. (1988) Induction of differentiation in neoplastic cells. Editorial commentary. In Oncology Overview: Selected Abstracts on Induction of Differentiation in Neoplastic Cells. CIDA-CCB Information Ventures, Inc., Philadelphia, Pa.

87. Andrews P. W. (1988) Human teratocarcinoma. Biochim. Biophys. Acta 948: 17-36.

88. Andrews P. W. and Oliver R. T. D. (1990) (Editors) Germ Cell Tumours of the Testis: Cancer Surveys 9 [Editorial Commentary, pp 239-241].

89. Andrews P. W., Marrink J., Hirka G., von Keitz A., Sleijfer D. and Gönczöl E. (1991) The surface antigen phenotype of human embryonal carcinoma cells: Modulation upon differentiation and viral infection. In: Recent Results in Cancer Research, Vol 123; Pathobiology of Human Germ Cell Neoplasia (J. W. Oosterhuis, H. Walt and I. Damjanov, eds.). Springer-Verlag, pp 63-83.

90. Bottero L., Simeone A., Arcioni L., Acampora D., Andrews P. W., Boncinelli E. and Mavilio F. (1991) Differential activation of homeobox genes by retinoic acid in human embryonal carcinoma cells. In: Recent Results in Cancer Research, Vol 123; Pathobiology of Human Germ Cell Neoplasia (J. W. Oosterhuis, H. Walt and I. Damjanov, eds.). Springer-Verlag, pp 133-143.

91. McCarrick J. and Andrews P. W. (1992) Embryonal carcinoma cells and embryonic stem cells as models for neuronal development and function. In: Cell Lines in Neurobiology: A Practical Approach (J. Wood, ed.). IRC Press, Oxford pp 77-104.

92. Fenderson B. A. and Andrews P. W. (1992) Carbohydrate antigens of embryonal carcinoma cells; changes upon differentiation. Acta Path. Microbiol. Immunol. Scand. Vol. 100, Suppl. 27 “Carbohydrate Pathology”. (Dabelsteen, E. & Clausen, H., eds), Munksgaard Copenhagen, pp 109-118.

93. Andrews, P. W. (1993). Teratomas—the cross roads of embryology and oncology. Oncology Newsletter (Journal of the Yorkshire Regional Cancer Organisation), No. 14 pp 16-17.

94. Andrews P. W., Damjanov I. (1994) Cell lines from human germ cell tumors. In: Atlas of Human Tumor Cell Lines (R. J. Hay, J-G Park, A. Gazdar, eds.). Academic Press, pp 443476.

95. Oosterhuis, J. W., Andrews, P. W. (1996). Differentiation in germ cell tumours. In: Testicular Cancer (2nd Edition) (A. Horwich, ed) Chapman & Hall, pp 61-72.

96. Andrews, P. W., Wakeman, J. (1996). Cell differentiation in germ cell tumours. In: “Ares Serono Conference on Sex Differentiation” (I. A. Hughes, ed), Frontiers in Endocrinology vol. 20, pp 3344

97. Andrews, P. W., (1998) Teratocarcinomas and human embryology: pluripotent human EC cell lines. Acta Pathologica Microbiologica et Immunologica Scandinavica, 106:158-168.

98. Gokhale, P. J., Eastwood, D., Walsh, J., Andrews, P. W. (1998). The possible role of Notch genes in Germ Cell Tumour Development and Progression. Germ Cell Tumours IV (W G Jones, I Appleyard, P Handen & J K Joffee, eds), John Libby, London, pp 69-71.

99. Andrews, P. W. (2001) Life story inside a cell. Times Higher Education Supplement. Jan. 19th 2001, p21.

100. Andrews, P. W., Przyborski, S. A. and Thomson, J. A. (2000). Embryonal Carcinoma Cells as Embryonic Stem Cells. Cold Spring Harbor Laboratory Press. In press.

1. A method to modulate the differentiation of an embryonic stem cell comprising:
i) providing a culture of embryonic stem cells;
ii) providing at least one ligand, or the active binding fragment thereof, capable of binding its cognate receptor polypeptide expressed by said embryonic stem cell;
iii) forming a culture comprising embryonic stem cells and said ligand; and
iv) growing said cell culture.

2. A method according to claim 1 wherein said ligand is encoded by a nucleic acid molecule selected from the group consisting of:
i) a nucleic acid molecule as represented in FIG. 22;
ii) a nucleic acid molecule which hybridises to the nucleic acid in (i) and which encodes a ligand capable of binding a Wnt receptor; and
iii) nucleic acid molecules which are degenerate as a result of the genetic code to the sequences defined in (i) and (ii) above.

3. A method according to claim 2 wherein said ligand is encoded by a nucleic acid molecule selected from the nucleic acid sequences represented in: FIG. 30; FIG. 32; FIG. 34; FIG. 36; FIG. 38; FIG. 40; FIG. 42; FIG. 44; FIG. 47; FIG. 49; FIG. 51; FIG. 53; FIG. 55.

4. A method according to claim 2 or 3 wherein said ligand is encoded by a nucleic acid molecule as represented by the nucleic acid sequence in FIG. 22.

5. A method according to claim 1 wherein said ligand is encoded by a nucleic acid molecule selected from the group consisting of:
i) a nucleic acid molecule as represented in FIGS. 2, 4, 5, 7, 10, 12, 14, 16, or 18.
ii) a nucleic acid molecule which hybridises to the nucleic acid in (i) and which encodes a ligand capable of modulating embryonic stem cell differentiation; and
iii) nucleic acid molecules which are degenerate as a result of the genetic code to the sequences defined in (i) and (ii) above.

6. A method according to claim 5 wherein said ligand is selected from the group comprising the amino acid sequences in FIGS. 3, 6, 8, 9, 11, 13, 15, 17, 19, or polypeptide variants thereof.

7. A method according to any of claims 1-6 wherein said cells are induced to differentiate by the addition of at least one agent selected from the group consisting of: retinoic acid; hexamethylene bisacetamide; bone morphogenetic proteins; bromodeoxyuridine; lithium; sonic hedgehog.

8. A method for modulating the differentiation of embryonic stem cells comprising:
i) providing a cell transfected with a nucleic acid molecule selected from the group consisting of.
a) a nucleic acid molecule as represented in FIGS. 2, 4, 5, 7, 10, 12, 14, 16, 18.
b) a nucleic acid molecule which hybridises to the nucleic acid in (ii) and which encodes a ligand capable of modulating embryonic stem cell differentiation; and
c) nucleic acid molecules which are degenerate as a result of the genetic code to the sequences defined in (a) and (b) above.
ii) forming a culture comprising the cell identified in (i) above with an embryonic stem cell; and
iii) growing said culture under conditions suitable for the maintenance and/or differentiation of said embryonic stem cell.

9. A method for modulating the differentiation of embryonic stem cells comprising:
i) providing a cell transfected with a nucleic acid molecule selected from the group consisting of:
a) a nucleic acid molecule as represented by the sequence in FIG. 22;
b) a nucleic acid molecule which hybridises to the nucleic acid in (a) and which encodes a ligand capable of binding a Wnt receptor; and
c) nucleic acid molecules which are degenerate as a result of the genetic code to the sequences defined in (a) and (b) above.
ii) forming a culture comprising a cell identified in (i) above with an embryonic stem cell; and
iii) growing said culture under conditions suitable for the maintenance and/or differentiation of embryonic stem cells.

10. A method according to claim 9 wherein said cell expresses Wnt-13 ligand.

11. A method according to any of claims 9 or 10 wherein said cells are induced to differentiate by the addition of at least one agent selected from the group consisting of: retinoic acid; hexamethylene bisacetamide; bone morphogenetic proteins; bromodeoxyuridine; lithium; sonic hedgehog.

12. A method according to any of claims 1-11 wherein said nucleic acid molecule encodes a ligand of human origin.

13. A method according to any of claims 1-12 wherein said embryonic stem cells are of human origin.

14. A method according to any of claims 8-13 wherein said transfected cell is a mammalian cell.

15. A cell according to claim 14 wherein said cell is selected from the group consisting of: a chinese hamster ovary cell; murine primary fibroblast cell; human primary fibroblast cell; transformed mouse fibroblast cell-line STO.

16. A method for inhibiting the differentiation of embryonic stem cells comprising the steps of:
i) providing at least one polypeptide, or active fragment thereof, wherein said polypeptide is an inhibitor of the Wnt signalling pathway.
iii) forming a culture comprising the polypeptide identified in (i) above with an embryonic stem cell; and
iii) growing said culture under conditions suitable for the maintenance of embryonic stem cells in an undifferentiated state.

17. A method according to claim 16 wherein said inhibitor is selected from the group consisting of the active binding fragments thereof of the following polypeptides: frizzled related polypeptides (FRP); Wnt Inhibitory Factors (WIF);
Dickkopf; Cerebrus.

18. A method according to claim 17 wherein said inhibitor is encoded by a nucleic acid molecule selected from the nucleic acid sequences represented by: FIG. 57; FIG. 59; FIG. 61; FIG. 63; FIG. 65; FIG. 67; FIG. 69; FIG. 71; FIG. 73; FIG. 75; FIG. 77; FIG. 79; FIG. 81; FIG. 83; FIG. 85; FIG. 87; FIG. 89; FIG. 91; FIG. 93; FIG. 95; FIG. 97; FIG. 99; FIG. 101; or FIG. 103.

19. A method for inhibiting the differentiation of embryonic stem cells comprising the steps of:
i) providing a cell transfected with a nucleic acid molecule selected from the group consisting of:
a) a nucleic acid molecule encoding a Wnt inhibitory polypeptide;
b) a nucleic acid molecule which hybridises to the nucleic acid in (a) and which encodes a polypeptide capable of inhibiting Wnt signalling; and
c) nucleic acid molecules which are degenerate as a result of the genetic code to the sequences defined in (a) and (b) above.
ii) forming a culture of the cell identified in (i) above with an embryonic stem cell; and
iii) growing said culture under conditions suitable for the maintenance of embryonic stem cells in an undifferentiated state.

20. A method according to claim 19 wherein said cells express at least one Wnt inhibitory polypeptide selected from the group consisting of the active binding fragments thereof of the following polypeptides: frizzled related polypeptides (FRP); Wnt Ihibitory Factors (WI); Dickkopf; Cerebrus.

21. A method according to claim 19 wherein said cells express at least one Wnt inhibitory polypeptide encoded by a nucleic acid molecule selected from the nucleic acid sequences represented by: FIG. 57; FIG. 59; FIG. 61; FIG. 63; FIG. 65; FIG. 67; FIG. 69; FIG. 71; FIG. 73; FIG. 75; FIG. 77; FIG. 79; FIG. 81; FIG. 83; FIG. 85; FIG. 87; FIG. 89; FIG. 91; FIG. 93; FIG. 95; FIG. 97; FIG. 99; FIG. 101; Fig or 103.

22. A cell or cell culture obtainable by the method according to any of claims 1-21.

23. A therapeutic cell composition obtainable by the method according to any of claims 1-15.

24. Use of a cell according to claim 23 for the manufacture of a composition for use in the treatment of a disease selected from the group consisting of Parkinson's disease; Huntington's disease; motor neurone disease; heart disease; diabetes; liver disease (eg cirrhosis); renal disease; AIDS.

25. A method of treatment of an animal, preferably a human, comprising administering a cell composition comprising embryonic stem cells which have been induced to differentiate into at least one cell-type by the method according to any of claims 1-14.

26. Condition medium obtained by culturing embryonic stem cells according to the method of any of claims 1-21.
